(19) United States
(12) Patent Application Publication
(10) Pub. No.: US 2023/0059649 A1
(43) Pub. Date: Feb. 23, 2023

(54) TEMPERATURE-BASED TRANSIENT DELIVERY OF ZSCAN4 NUCLEIC ACIDS AND PROTEINS TO CELLS AND TISSUES

(71) Applicant: FLIXIRGEN THERAPEUTIC, INC., Baltimore, MD (US)

(72) Inventor: Minoru S.H. KO, Baltimore, MD (US)

(21) Appl. No.: 17/789,142

(22) PCT Filed: Dec. 30, 2020
(86) PCT No.: PCT/US20/67507

§ 371 Date: Jun. 24, 2022

Related U.S. Application Data
(60) Provisional application No. 62/955,820, filed on Dec. 31, 2019.
(60) Provisional application No. 62/992,745, filed on Mar. 20, 2020.

(51) Int. Cl. C12N 5/0789; C12N 15/86; A61P 43/00

(52) U.S. Cl. CPC C12N 5/0647; C12N 15/86; A61P 43/00; C12N 2760/18843; C12N 2510/00; C12N 2501/22

(57) ABSTRACT

The present disclosure relates to methods for transiently activating temperature-sensitive agents in one or more cells, for example by contacting one or more cells with a temperature-sensitive agent and transiently incubating the cells at a permissive temperature for inducing an activity of the temperature-sensitive agent in the cells. Additionally, the present disclosure relates to methods of contacting one or more cells in a subject with a temperature-sensitive agent and then lowering the subject's body temperature to a permissive temperature for inducing an activity of the temperature-sensitive agent in the cells. The disclosure also relates to methods of treating a subject with a temperature-sensitive therapeutic agent. In particular, the disclosure provides tools for temperature-sensitive delivery of ZSCAN4 nucleic acids and proteins to cells.

### CROSS-REFERENCE TO RELATED APPLICATIONS

This application claims the benefit of U.S. Provisional Application No. 62/992,745, filed Mar. 20, 2020, and U.S. Provisional Application No. 62/955,820, filed Dec. 31, 2019, the disclosures of which are hereby incorporated by reference in their entirety.

### SUBMISSION OF SEQUENCE AS ASCII TEXT FILE

The content of the following submission on ASCII text file is incorporated herein by reference in its entirety: a computer readable form (CRF) of the Sequence Listing (file name: 699442001340SEQLIST.TXT, date recorded: Dec. 23, 2020, size: 25 KB).

### FIELD

The present disclosure relates to methods for transiently activating a temperature-sensitive agent (ts-agent) in one or more cells, for example by contacting one or more cells with a ts-agent and transiently incubating the cells at a permissive temperature for inducing an activity of the ts-agent in the cells. For ex vivo therapeutic strategies, one or more cells are treated with a therapeutic ts-agent ex vivo at the permissive temperature and the cells are subsequently transferred to a subject at the non-permissive temperature (e.g., the subject's normal core body temperature). For in vivo therapeutic strategies, a therapeutic ts-agent is delivered to a subject that is maintained at the permissive temperature, permitting the therapeutic ts-agent to function in vivo for a limited time before the ts-agent is turned off permanently when the subject's body temperature returns to normal, or when the subject's surface body temperature is raised (e.g., the non-permissive temperature). Alternatively, a therapeutic ts-agent is delivered to a subject and the ts-agent is subsequently transiently activated by lowering the subject's core body temperature to a permissive temperature for inducing an activity of the therapeutic ts-agent in cells of the subject. In particular, the present disclosure provides tools for temperature-sensitive delivery of ZSCAN4 nucleic acids and proteins to cells.

### BACKGROUND

Delivery of a therapeutic gene product to human cells, tissues, and organs poses a great challenge. For traditional gene therapy, which requires the continuous expression of a gene to supplement the defect of the gene in a patient, this has been achieved by using a viral vector such as a retrovirus, a lentivirus, an adenovirus, or an adeno-associated virus. However, an equally important strategy gene therapy involves transient, short-term expression of a gene. For such applications, the persistent expression of a gene is not required and may actually be deleterious to the cells.

For example, CAS9 is a bacterial enzyme that cleaves DNA. It is an important component of CRISPR/CAS9-based gene editing complex, which has been considered for gene therapy. Both guide RNA and CAS9 can be encoded by genes on a single Sendai virus vector (Park et al., 2016). In order to use the gene-editing system therapeutically, vectors containing CRISPR-CAS9 must be introduced into human cells or the human body. However, the continuous expression of CAS9 could cause the introduction of DNA breaks and mutations. Thus, it is desirable to have CAS9 expressed for a short period of time, for example, on the order of hours or a few days, rather than a week or more.

Another application for short-term expression of a gene is for cellular reprogramming. Recently, it has been shown that the ectopic expression of a set of transcription factors can convert cells into therapeutically useful cell types. For example, a set of three transcription factors can convert pancreatic duct cells into insulin-secreting pancreatic beta-cells (Zhou et al., 2008). Another set of transcription factors can convert fibroblast cells into cardiomyocytes (Ieda et al., 2010). It is thought that in vivo delivery of these transcription factors into the human body could be used as one type of regenerative medicine. However, it is desirable to have these potent cell identity-changing transcription factors expressed only transiently, as the continuous expression of these potent transcription factors may cause harm.

As the above examples highlight, traditional gene therapy using viral vectors that lead to the continuous expression of a gene can be undesirable. For time-limited expression of a gene product, the delivery of synthetic or in vitro-transcribed mRNA into cells has begun to be used (Warren et al., 2010). However, there are several problems with these methodologies. For example, the amount of mRNA delivered to cells, tissues, and organs is limited, and thus, the amount of protein product may not be sufficient for biologically meaningful effects in vivo.

Also, due to the fast turn-over of RNA, which normally lasts for only up to 12 hours (Warren et al., 2010; Goparaju et al., 2017), synthetic RNA must be transfected into cells multiple times. For the forced differentiation of human pluripotent stem cells such as embryonic stem cells and induced pluripotent stem (iPS) cells, twice-daily transfections over the course of several days are required (Akiyama et al., 2016; Goparaju et al. 2017). To generate iPS cells from human fibroblast cells, daily transfection of a cocktail of synthetic RNAs must continue for more than two weeks (Warren et al., 2010). This is not only cumbersome, but also inefficient.

For the generation of iPS cells, this issue has been addressed by using self-replicating RNA, which enables long-term expression after only one delivery (Yoshioka et al., 2013). Self-replicating RNAs are single-stranded RNAs that are usually produced from alphaviruses (Jose et al., 2009), such as Venezuelan Equine Encephalitis Virus (VEEV), Sindbis Virus (SINV), and Semliki Forest Virus (SFV), by removing DNA encoding structural proteins that are required for virus particle formation (Petrakova et al., 2005). Self-replicating RNAs encode nonstructural proteins (nsPs), which function as an RNA-dependent RNA polymerase to replicate the self-replicating RNA itself and to produce a transcript for translation. Self-replicating RNAs can also include a gene of interest (GOI) encoding a protein of interest, and other genetic elements. Due to its positive feedback production of RNAs, self-replicating RNAs can express the GOI at a high level. Self-replicating RNAs can be delivered to mammalian cells as a naked RNA (i.e., a synthetic RNA) or as a virus particle, which can be generated by supplementing the missing virus structural proteins by packaging helper cells.

The advantage of self-replicating RNA vectors are their self-replicating feature, which results in enhancement of expression levels of a GOI. However, one of the drawbacks of self-replicating RNA vectors to deliver RNA/protein to mammalian cells is their persistent expression. Usually, a positive feedback production of an RNA-dependent RNA polymerase and a GOI continues, which may result in the death of cells transfected with a naked RNA form of the self-replicating RNA or infected with a viral form of the self-replicating RNA.

Thus, what is needed in the art of gene therapy are tools for the transient expression of a GOI encoding a protein of interest, such as a therapeutic agent (e.g., human ZSCAN4). In particular, control of transcription and translation of RNA vectors and self-replicating RNA is desirable.

### SUMMARY

Based on the necessity of having time-limited expression of a gene of interest (GOI), a transient gene product delivery system is required, where a nucleic acid or protein can be delivered to or expressed in specific cells, in vitro or in vivo, where the amount of nucleic acid/protein is sufficient to have a biologically meaningful effect, and where transient expression can be turned off permanently after achieving the biologically meaningful effect. In order to meet these and other needs, the present disclosure relates to tools for transiently inducing an activity of a temperature-sensitive agent (ts-agent) such as a therapeutic ts-agent, either in a subject (in vivo) or in cells in culture (ex vivo). In some embodiments, the therapeutic ts-agent is used in combination with mild therapeutic hypothermia. In other embodiments, the therapeutic ts-agent is used in combination with mild therapeutic hyperthermia, or a localized application of heat. In some embodiments, the ts-agent is a ts-RNA molecule or ts-protein molecule. In some embodiments, the ts-agent is encoded by a heterologous nucleic acid inserted in a temperature-sensitive viral vector or a self-replicating RNA. In some embodiments, the viral vector is selected from but not limited to a Sendai virus vector, a retrovirus vector, an adeno virus vector, an adeno-associated virus vector, and an Alpha virus vector. In some embodiments, the self-replicating RNA comprises an Alphavirus replicon lacking a viral structural protein coding region. In some embodiments, the Alphavirus is selected from but not limited to a Venezuelan equine encephalitis virus, a Sindbis virus, and a Semliki Forrest virus. A gene product of particular interest is ZSCAN4, particularly human ZSCAN4.

The foregoing and other objects and features of the disclosure will become more apparent from the following detailed description, which proceeds with reference to the accompanying figures.

### BRIEF DESCRIPTION OF THE DRAWINGS

FIGS. 1A-1D depict the structure of the Venezuelan Equine Encephalitis Virus (VEEV) genome and locations of mutated regions. FIG. 1A shows a schematic representation of a wild type VEEV genome (TC-83 strain: complete genome 11,446 bp linear RNA: NCBI Accession: L01443.1 GI: 323714). Genes of nonstructural proteins (nsP1, nsP2, nsP3, nsP4) encode RNA-dependent RNA polymerase and genes of structural proteins encode viral envelope proteins (C, E1, E2). 5′-UTR (5′-untranslated region) and 3′-UTR (3′-untranslated region). The gene of the nsP2 protein, presented as a bold box, was mutated to produce temperature sensitivity. FIG. 1B shows a schematic representation of nsP2 with mutation 1 (temperature-sensitive mutant 1: ts1). Five amino acids were inserted between amino acids 439 and 440. FIG. 1C shows a schematic representation of nsP2 with mutation 2 (ts2). Five amino acids were inserted between amino acids 586 and 587. FIG. 1D shows a schematic representation of nsP2 with mutation 3 (ts3). Five amino acids were inserted between amino acids 594 and 595.

FIGS. 2A-2C depict partial sequences of VEEV nsP2, corresponding to the regions mutated in ts1, ts2, and ts3. FIG. 2A shows the wild type sequence in comparison to mutant 1 (ts1), which includes a 15 nucleotide insertion resulting in a 5 amino acid insertion. FIG. 2B shows the wild type sequence in comparison to mutant 2 (ts2), which includes a 15 nucleotide insertion resulting in a 5 amino acid insertion. FIG. 2C shows the wild type sequence in comparison to mutant 3 (ts3), which includes a 15 nucleotide insertion resulting in a 5 amino acid insertion.

FIG. 3 depicts partial nucleotide sequences for VEEV nsP1 of wild type (TC-83 strain) and mutant 4 (ts4), set forth as SEQ ID NO:19 and SEQ ID NO:20, respectively. The 5′-UTR and the 51-nt CSE (conserved sequence element) are shown in bold. Mutated nucleotides in ts4 are underlined.

FIGS. 4A and 4B depict testing temperature-sensitivity of srRNA1ts2 and srRNA1ts3 at 30° C., 32° C., and 37° C. Wild type (srRNA1wt-GFP) and mutant (srRNA1ts2-GFP, srRNA1ts3-GFP) self-replicating RNA (srRNA) vectors were generated. RNAs produced by in vitro transcription were transfected into human induced pluripotent stem cells (ADSC-iPSC line). Cells were cultured in CO₂ incubators maintained at 30° C., 32° C., and 37° C., respectively. Pictures of cells were obtained at 20 hours and 48 hours, respectively. The upper panels show phase-contrast images and the lower panels show fluorescence images detecting expression of green fluorescence protein (GFP). FIG. 4A shows results from transfection of cells with srRNA1wt-GFP, srRNA1ts2-GFP, and srRNA1ts3-GFP RNA. FIG. 4B shows results from transfection of cells with synthetic mRNA encoding GFP (synRNA-GFP).

FIG. 5 depicts testing temperature-sensitivity of srRNA1ts1 and srRNA1ts2 at 32° C. Wild type (srRNA1wt-GFP) and mutant (srRNA1ts2-GFP and srRNA1ts1-GFP) self-replicating RNA (srRNA) vectors were generated. RNAs produced by in vitro transcription were transfected into human induced pluripotent stem cells (ADSC-iPSC line). Cells were cultured in CO₂ incubators maintained at 32° C. Pictures of cells were obtained at 24, 48, 72, 96, 120, 144, 168, 192, 240, 288 hours. For the srRNA1ts1-GFP, only pictures of 24 hours and 168 hours were taken. The upper panels show phase-contrast images and the lower panels show fluorescence images detecting expression of GFP.

FIG. 6 depicts testing temperature-sensitivity of srRNA1ts2 and srRNA1ts4 at 32° C., 33° C., 37° C. Mutant (srRNA1ts2-GFP and srRNA1ts4-GFP) self-replicating RNA (srRNA) vectors were generated. RNAs produced by in vitro transcription were transfected into human induced pluripotent stem cells (ADSC-iPSC line). Cells were cultured in CO₂ incubators maintained at 32° C., 33° C., 37° C., respectively. Pictures of cells were obtained at 20, 48, 96 hours. The upper panels show phase-contrast images and the lower panels show fluorescence images detecting expression of GFP.

FIG. 7 depicts testing temperature-sensitivity of mutant srRNA1ts2-GFP maintained at 32° C. RNAs produced by in vitro transcription of a mutant vector (srRNA1ts2-GFP) were transfected into human induced pluripotent stem cells (ADSC-iPSC line). Cells were cultured in CO₂ incubators maintained at 32° C. The srRNA1ts2-GFP vector contains a puromycin N-acetyltransferase (pac) selection gene inserted after the “IRES” sequence, and thus, transfected cells can be selected using puromycin. The experiments were done in the absence (upper panel) or presence (lower panel) of 1 pg/ml of puromycin. Pictures of cells were obtained at 24, 48, 96, 144, 168, 192 hours. The upper panels show phase-contrast images and the lower panels show fluorescence images detecting expression of GFP.

FIG. 8 depicts testing temperature-sensitivity of mutant srRNA1ts2-GFP with a temperature switch from 32° C. to 37° C. at 24 hours. RNAs produced by in vitro transcription of a mutant vector (srRNA1ts2-GFP) were transfected into human induced pluripotent stem cells (ADSC-iPSC line). Cells were cultured in CO₂ incubators maintained at 32° C. At 24 hours, cells were transferred to a CO₂ incubator maintained at 37° C. The srRNA1ts2-GFP vector contains a puromycin N-acetyltransferase (pac) selection gene inserted after the “IRES” sequence, and thus, transfected cells can be selected using puromycin. The experiments were done in the absence (upper panel) or presence (lower panel) of 1 μg/ml of puromycin. Pictures of cells were obtained at 24, 48, 96, 144, 168, 192 hours. The upper panels show phase-contrast images and the lower panels show fluorescence images detecting expression of GFP.

FIG. 9 depicts testing temperature-sensitivity of mutant srRNA1ts2-GFP with a temperature switch from 32° C. to 37° C. at 48 hours. RNAs produced by in vitro transcription of a mutant vector (srRNA1ts2-GFP) were transfected into human induced pluripotent stem cells (ADSC-iPSC line). Cells were cultured in CO₂ incubators maintained at 32° C. At 48 hours, cells were transferred to CO₂ incubator maintained at 37° C. The srRNA1ts2-GFP vector contains a puromycin N-acetyltransferase (pac) selection gene inserted after the “IRES” sequence, and thus, transfected cells can be selected using puromycin. The experiments were done in the absence (upper panel) or presence (lower panel) of 1 μg/ml of puromycin. Pictures of cells were obtained at 24, 48, 96, 144, 168, 192 hours. The upper panels show phase-contrast images and the lower panels show fluorescence images detecting expression of GFP.

FIG. 10 depicts testing temperature-sensitivity of mutant srRNA1ts2-GFP with a temperature switch from 32° C. to 37° C. at 72 hours. RNAs produced by in vitro transcription of a mutant vector (srRNA1ts2-GFP) were transfected into human induced pluripotent stem cells (ADSC-iPSC line). Cells were cultured in CO₂ incubators maintained at 32° C. At 72 hours, cells were transferred to a CO₂ incubator maintained at 37° C. The srRNA1ts2-GFP vector contains a puromycin N-acetyltransferase (pac) selection gene inserted after the “IRES” sequence, and thus, can be selected using puromycin. The experiments were done in the absence (upper panel) or presence (lower panel) of 1 μg/ml of puromycin. Pictures of cells were obtained at 24, 48, 96, 144, 168, 192 hours. The upper panels show phase-contrast images and the lower panels show fluorescence images detecting expression of GFP.

FIGS. 11A-11D depict testing temperature-sensitivity of mutant srRNA1ts2-GFP in fibroblast cells. RNAs produced by in vitro transcription of a mutant vector (srRNA1ts2-GFP) were transfected into human newborn dermal fibroblast cells (HDFn line). Cells were cultured in CO₂ incubators maintained at 32° C. Pictures of cells were obtained at 24, 48, and 96 hours. The upper panels show phase-contrast images and the lower panels show fluorescence images detecting expression of GFP. FIGS. 11A and 11B depict transfections carried out using JetMessenger (Polyplus). Cells were cultured in standard media alone (FIG. 11A) or standard media supplemented with 200 ng/ml of B18R (FIG. 11B). FIGS. 11C and 11D depict transfections carried out using MessengerMax (ThermoFisher). Cells were cultured in standard media alone (FIG. 11C) or standard media supplemented with 200 ng/ml of B18R (FIG. 11D).

FIG. 12 depicts an alignment of amino acid sequences corresponding to nsP2 mutant 2 (ts2) of various alphavirus family members. The left panel depicts an alignment (reproduced in part from FIG. 1 of Russo et al., 2006) of wild type sequences set forth as SEQ ID NOS:21-28, while the right panel depicts an alignment of mutant sequences set forth as SEQ ID NOS:29-36 including an insertion of 5 amino acids between the “β5” and “β6” (5^{th} and 6^{th} “β strands) in the secondary structure of nsP2. VEEV (Venezuelan equine encephalitis virus), Aura (Aura virus), WEEV (Western equine encephalitis virus), BFV (Barmah Forest virus), ONNV (O'nyong-nyong virus), RRV (Ross River virus), SFV (Semliki Forest virus), and SINV (Sindbis virus).

FIG. 13 depicts a schematic diagram showing a typical ex vivo treatment of cells with temperature-sensitive agents (ts-agents). Ts-agents such as srRNAs or Sendai virus vectors, are functional at a permissive temperature (e.g., 33° C.), but non-functional at a non-permissive temperature (e.g., 37° C.). Target cells treated with the ts-agent are cultured at a permissive temperature for a certain duration (e.g., 3 days), and then continue to be cultured at a non-permissive temperature for a certain duration (e.g., 10 days). Expected levels of RNA (or protein translated from the RNA) of a gene of interest (GOI) increase at a permissive temperature and reach a high level. After switching to a non-permissive temperature, expected levels of RNA (or proteins) gradually decrease as transcription and translation cease.

FIG. 14 depicts a schematic diagram showing an exemplary ex vivo therapeutic procedure. Temperature-sensitive agents (ts-agents) such as srRNAs or Sendai virus vectors, are functional at a permissive temperature (e.g., 33° C.), but non-functional at a non-permissive temperature (e.g., 37° C.). Target cells are taken from a patient's body (autograft) and are incubated with the ts-agent ex vivo at a permissive temperature, e.g., at 33° C., for a certain duration, e.g., 24 hours. Then, the target cells with ts-agents are transplanted in the patient. At a non-permissive temperature of 37° C., the ts-agent does not function inside the patient's body.

FIG. 15 depicts a schematic diagram showing another exemplary ex vivo therapeutic procedure. Temperature-sensitive agents (ts-agents) such as srRNAs or Sendai virus vectors, are functional at a permissive temperature (e.g., 33° C.), but non-functional at a non-permissive temperature (e.g., 37° C.). Target cells are taken from a donor's body (allograft) and are incubated with the ts-agent ex vivo at a permissive temperature, e.g., at 33° C., for a certain duration, e.g., 24 hours. Then, the target cells with ts-agents are transplanted in a patient. At a non-permissive temperature of 37° C., the ts-agent does not function inside the patient's body.

FIG. 16 depicts a schematic diagram showing an exemplary semi in vivo therapeutic procedure. Temperature-sensitive agents (ts-agents) such as srRNAs or Sendai virus vectors, are functional at a permissive temperature (e.g., 33° C.), but non-functional at a non-permissive temperature (e.g., 37° C.). A patient undergoes a procedure for therapeutic hypothermia and the patient's core body temperature is maintained at a reduced temperature (e.g., 33° C.), which is lower than normal body temperature (e.g., 37° C.). Target cells (either autologous or allogenic) are treated with the ts-agent ex vivo and immediately infused into the patient's circulation or injected into an organ of the patient. While the patient is maintained at the reduced temperature (e.g., 33° C.) for some time (e.g., 24 hours) the ts-agent is functional. Subsequently, the patient's core body temperature is returned to normal temperature (37° C.), at which time the ts-agent is no longer functional.

FIG. 17 depicts a schematic diagram showing an exemplary in vivo therapeutic procedure. Temperature-sensitive agents (ts-agents) such as srRNAs or Sendai virus vectors, are functional at a permissive temperature (e.g., 33° C.), but non-functional at a non-permissive temperature (e.g., 37° C.). A patient undergoes a procedure for therapeutic hypothermia and the patient's core body temperature is maintained at a reduced temperature (e.g., 33° C.), which is lower than normal body temperature (e.g., 37° C.). The ts-agent is directly delivered systemically or to specific organs, tissues, or cell types. While the patient is maintained at the reduced temperature (e.g., 33° C.) for some time (e.g., 24 hours), the ts-agent is functional. Subsequently, the patient's core body temperature is returned to normal temperature (37° C.), at which time the ts-agent is no longer functional.

FIG. 18 shows a schematic representation of an exemplary temperature sensitive-Sendai virus vector that includes the coding region (open reading frame) of human ZSCAN4 (SeV18+hZscan4/TS15ΔF; also called “SeVts-ZSCAN4”). The vector backbone, which has been described as TS15 (Ban et al., 2011), lacks the F (fusion) gene required to reproduce infectious progeny virus. Thus, this vector does not transmit virus from infected cells to uninfected cells. This vector encodes two RNA polymerase genes (P and L), and three structural protein genes (NP, M and HN), and contains point mutations in the M, HN, P and L genes, which makes the vector temperature-sensitive: permissive at 33° C.; non-permissive above 37° C. To construct SeVts-ZSCAN4, the coding region of the human ZSCAN4 gene was inserted upstream of NP gene in the TS15 vector backbone, the location of which provides the highest expression among genes in the Sendai virus genome.

FIG. 19 indicates that the majority of human CD34+ cells contacted with SeVts-ZSCAN4 and incubated at the permissive temperature (e.g., 33° C.) for 16 or 24 hours express ZSCAN4 protein.

FIGS. 20A and 20B indicate that human CD34+ cells contacted with SeVts-ZSCAN4 and incubated at the permissive temperature (e.g., 33° C.) express ZSCAN4 protein, but begin to lose ZSCAN4 protein expression when subsequently incubated at the non-permissive temperature (e.g., 37° C.).

FIG. 21 indicates that the telomeres of human CD34+ cells contacted with SeVts-ZSCAN4 and incubated at the permissive temperature (e.g., 33° C.) for as little as 24 hours are extended.

FIG. 22 depicts a schematic diagram showing the ex vivo procedure of Example 14, in which human CD34+ cells are contacted with a ZSCAN4 therapeutic ts-agent at a permissive temperature (e.g., 33° C.), and are subsequently infused into an immune compromised mouse having a non-permissive normal body temperature (e.g., 37° C.) to assess safety and efficacy of CD34+ cell engraftment.

FIG. 23 indicates that SeVts-ZSCAN4 treatment for 24 hours at the permissive temperature (e.g., 33° C.) was effective in extending telomeres of human CD34+ cells in vitro.

FIG. 24 indicates that telomeres of human cells engrafted in immune comprised mice were longer if the human CD34+ cells injected into the mice were first contacted with SeVts-ZSCAN4 and incubated at the permissive temperature (e.g., 33° C.) in vitro. Mouse 492 and Mouse 493 received SeVts-ZSCAN4-contacted CD34+ cells, whereas Mouse 496 received CD34+ cells that were not contacted with SeVts-ZSCAN4.

FIG. 25 depicts a schematic diagram showing the ex vivo therapeutic procedure of Example 15, in which autologous CD34+ cells are contacted with a ZSCAN4 therapeutic ts-agent at a permissive temperature (e.g., 33° C.), and are subsequently infused into a patient having a non-permissive normal body temperature (e.g., 37° C.).

FIG. 26 shows a flow chart of the clinical procedure of Example 15, for evaluation of autologous CD34+ cells contacted with SeVts-ZSCAN4 in human patients with telomere biology disorders and bone marrow failure.

FIG. 27 depicts another schematic diagram showing the ex vivo therapeutic procedure of Example 15, for evaluation of autologous CD34+ cells contacted with SeVts-ZSCAN4 in human patients with telomere biology disorders and bone marrow failure.

FIG. 28 depicts a schematic diagram showing an exemplary in vivo therapeutic procedure. Temperature-sensitive agents (ts-agents) such as srRNAs or Sendai virus vectors, are functional at a permissive temperature (e.g., 31-34° C.), but non-functional at a non-permissive temperature (e.g., >37° C.). The temperature at or just below the surface of a patient's body (surface body temperature), which is around 31-34° C., is lower than the core body temperature of the patient, which is around 37° C. The ts-agent is directly delivered by intradermal, subcutaneous, or intramuscular administration to a patient, where it is functional at the patient's surface body temperature. No further action is required. Alternatively, when the function of the ts-agent is no longer needed, the ts-agent can be rendered non-functional by transiently increasing the patient's surface body temperature.

FIG. 29 depicts a schematic diagram showing an exemplary in vivo therapeutic procedure. Temperature-sensitive agents (ts-agents) such as srRNAs or Sendai virus vectors, are functional at a permissive temperature (e.g., 31-35° C.), but non-functional at a non-permissive temperature (e.g., >37° C.). The temperature of airways of a patient's body (airway temperature), which is around 32° C. for nasal cavity and upper trachea, and 35° C. for subsegmental bronchi (McFadden et al., 1985), is lower than the core body temperature of the patient, which is around 37° C. The ts-agent is directly delivered by nasal administration (e.g., insufflation, inhalation or instillation) to a patient, where it is functional at the patient's airway temperature. No further action is required. When the function of the ts-agent is no longer needed, the ts-agent can be rendered non-functional by transiently increasing the patient's airway temperature.

### DETAILED DESCRIPTION

Overview

Applicant has demonstrated that cells can be cultured at a permissive temperature for inducing an activity of a temperature-sensitive therapeutic agent, and that the activity can lead to a therapeutic effect in the cells. Moreover, the activity of the temperature-sensitive therapeutic agent can be reduced or inhibited by subsequently incubating the cells at a non-permissive temperature. Applicant has also for the first time provided methods for use of temperature-sensitive agents (ts-agents) in vivo. The same types of ts-agents used in vitro can be used in vivo. For instance, after administration of a ts-agent to the core of a subject, the subject's core body temperature can be lowered to a permissive temperature for inducing an activity of the ts-agent. Alternatively, after administration of a ts-agent to the surface (epidermis, dermis, hypodermis, or skeletal muscle) of a subject, the subject's surface body temperature is maintained at a permissive temperature for inducing an activity of the ts-agent. The subject's surface body temperature may be maintained naturally or artificially. These methods provide new ways to deliver and transiently activate therapeutic agents such as nucleic acids and polypeptides. In particular, the present disclosure provides tools for temperature-sensitive delivery of ZSCAN4 nucleic acids and proteins to cells.

Accordingly, the present disclosure generally relates to methods of transiently inducing an activity of a temperature-sensitive agent (e.g., a temperature-sensitive therapeutic agent) in vitro. In some embodiments, one or more cells comprising a temperature-sensitive therapeutic agent are cultured at a permissive temperature for inducing an activity of the temperature-sensitive therapeutic agent. The cells are cultured at the permissive temperature for a period of time sufficient for the temperature-sensitive therapeutic agent to induce a therapeutic effect in the cells. The cells are then returned to a non-permissive temperature, wherein the non-permissive temperature reduces or inhibits an activity of the temperature-sensitive therapeutic agent. In another embodiment, the one or more cells do not already comprise a temperature-sensitive therapeutic agent, and are first contacted with a temperature-sensitive therapeutic agent. In some embodiments, after inducing a therapeutic effect in the one or more cells, the cells are administered to a subject in need thereof. In some embodiments, the one or more cells are isolated from a subject in need of treatment and after treating with a temperature-sensitive therapeutic agent, the cells are returned to said subject.

In another aspect, the present disclosure relates to methods of transiently inducing an activity of a temperature-sensitive agent (e.g., a temperature-sensitive therapeutic agent) in vivo. In some embodiments, one or more cells in a subject comprise a temperature-sensitive therapeutic agent, and the subject's body temperature is lowered to a permissive temperature for a period of time sufficient for the temperature-sensitive therapeutic agent to induce a therapeutic effect in the cells, and the subject's body temperature is then returned to normal body temperature. In another embodiment, the temperature-sensitive therapeutic agent is administered to the subject, either before or after the subject's body temperature is lowered to a permissive temperature.

Other aspects of the present disclosure relate to treating a disease or condition by mobilizing CD34+ cells from bone marrow of a subject suffering from the disease or condition (subject in need thereof), isolating the mobilized cells from the subject, incubating the isolated cells at a temperature of about 33° C.±0.5° C., contacting the cells with a temperature-sensitive viral vector, such as a Sendai virus vector, or a temperature-sensitive self-replicating RNA (srRNA), wherein the viral vector or the srRNA comprises a heterologous nucleic acid molecule encoding a protein of interest, maintaining the contacted cells at about 33° C.±0.5° C. for a sufficient period of time, wherein the viral vector or the srRNA is capable of replicating at 33° C.±0.5° C. and replication of the viral vector or the srRNA leads to increased expression of the heterologous nucleic acid molecule, and infusing the contacted cells into the subject thereby engrafting the contact cells and treating the disease or condition. Alternatively, after isolating the mobilized cells from the subject, the isolated cells are contacted with a temperature-sensitive viral vector, such as Sendai virus vector, or a temperature-sensitive srRNA before incubating the cells at a temperature of about 33° C.±0.5° C. In some embodiments, the disease or condition is a telomere biology disorder and the protein of interest is a ZSCAN4, such as human ZSCAN4.

In another aspect, the present disclosure relates to treating a disease or condition by administering to a subject suffering from the disease or condition (subject in need thereof) a temperature-sensitive viral vector, such as a Sendai virus vector, or a temperature-sensitive self-replicating RNA (srRNA), wherein the viral vector or the srRNA comprises a heterologous nucleic acid encoding a protein of interest, lowering the subject's core body temperature to about 33° C.±0.5° C., maintaining the subject's core body temperature at about 33° C.±0.5° C. for a sufficient period of time, wherein the viral vector or the srRNA is capable of replicating at 33° C.±0.5° C. and replication of the viral vector or the srRNA leads to increased expression of the heterologous nucleic acid, and allowing the subject's core body temperature to return to normal. Alternatively, lowering the subject's core body temperature to about 33° C.±0.5° C. is done prior administering a temperature-sensitive viral vector, such as a Sendai virus vector, or a temperature-sensitive srRNA. In some embodiments, the disease or condition is a telomere biology disorder and the protein of interest is a ZSCAN4, such as human ZSCAN4.

References and claims to methods for treating a disease or condition by administering a ts-agent or cells comprising the ts-agent to a subject, in their general and specific forms likewise related to:

a) the use of a ts-agent or cells comprising the ts-agent for the manufacture of a medicament for the treatment of a disease or condition; and

b) pharmaceutical compositions comprising a ts-agent or cells comprising the ts-agent for the treatment of a disease or condition.

In some embodiments of the methods of the proceeding paragraphs, the heterologous nucleic acid comprises a gene of interest (GOI) or otherwise encodes a protein of interest. Said another way, the heterologous nucleic acid comprises a coding region of a protein of interest. In preferred embodiments, the protein of interest is ZSCAN4, such as human ZSCAN4 or a variant thereof.

### Definitions

As used herein and in the appended claims, the singular forms “a,” “an” and “the” include plural forms unless otherwise indicated. For example, “a polynucleotide” includes one or more polynucleotides.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments. It is understood that aspects and embodiments described herein as “comprising” include “consisting of” and “consisting essentially of” embodiments.

The term “about” as used herein in reference to a value other than temperature, encompasses from 90% to 110% of that value (e.g., about 30 minutes refers to 27 min to 33 min), unless otherwise indicated. When use in reference to temperature in Celsius, about encompasses −1° C. to +1° C. of that value (e.g., about 37° C. refers to 36° C. to 38° C.), unless otherwise indicated. In contrast, the use of plus or minus without more, delineates the indicated range (e.g., 33° C.±0.5° C. refers to 32.5° C. to 33.5° C.

As used herein, numerical ranges are inclusive of the numbers defined the range (e.g., 12-18 nucleotides encompasses 12, 13, 14, 15, 16, 17 and 18 nucleotides).

The terms “isolated” and “purified” as used herein refers to an object (e.g., a cell) that is removed (e.g., separated) from its environment (e.g., cell culture, biological sample, etc.). “Isolated” objects are at least 50% free, preferably 75% free, more preferably at least 90% free, and most preferably at least 95% (e.g., 95%, 96%, 97%, 98%, or 99%) free from other components with which they are associated.

The terms “individual” and “subject” refer to mammals. “Mammals” include, but are not limited to, humans, non-human primates (e.g., monkeys), farm animals, sport animals, rodents (e.g., mice and rats) and pets (e.g., dogs and cats).

The term “dose” as used herein in reference to a pharmaceutical composition refers to a measured portion of the composition taken by (administered to or received by) a subject at any one time.

The term “treating” a disease or a condition refer to executing a protocol, which may include administering one or more pharmaceutical compositions to an individual (human or other mammal), in an effort to alleviate signs or symptoms of the disease. Thus, “treating” or “treatment” does not require complete alleviation of signs or symptoms, does not require a cure, and specifically includes protocols that have only a palliative effect on the individual. As used herein, and as well-understood in the art, “treatment” is an approach for obtaining beneficial or desired results, including clinical results. Beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total).

Temperature-Sensitive Agents

Certain aspects of the present disclosure relate to transiently inducing an activity of a temperature-sensitive agent (e.g., a temperature-sensitive therapeutic agent) in one or more cells. An activity of the temperature-sensitive agent refers to any desired activation, replication, or increased expression of the agent. As used herein, the term “temperature-sensitive agent” refers to any nucleic acid or polypeptide that has different levels of functionality at different temperatures. Exemplary temperature-sensitive agents include, without limitation, temperature-sensitive viral vectors, temperature-sensitive self-replicating RNAs, and temperature-sensitive polypeptides.

As used herein, the term “permissive temperature” refers to any temperature at which the activity of a temperature-sensitive agent of the present disclosure is induced. Typically, a permissive temperature is not the normal body temperature of a subject. The normal body temperature of a human subject is about 37° C.±0.5° C. Depending on the temperature-sensitive agent, a permissive temperature may be a temperature that is higher or lower than the normal body temperature of a subject. In some aspects, the permissive temperature for the temperature-sensitive agent ranges from 30° C. to 36° C. In some embodiments, the permissive temperature is from about 31° C. to about 35° C., or 32° C. to 34° C. (33° C.±1.0° C.). In some preferred embodiments, the permissive temperature is 33° C.±0.5° C. It follows that in some embodiments, the non-permissive temperature for the temperature-sensitive self-replicating RNAs of the present disclosure is above 36° C. In some preferred embodiments, the non-permissive temperature is 37° C.±0.5° C.

In some embodiments, the activity of the temperature-sensitive agent induced at a permissive temperature is reduced or inhibited at a non-permissive temperature. The term “non-permissive temperature”, as used herein, refers to any temperature at which an activity of a temperature-sensitive agent of the present disclosure is not induced. A temperature-sensitive agent is not induced when an activity of the temperature-sensitive agent is at least 95% less, at least 90% less, at least 85% less, at least 80% less, at least 75% less, or at least 50% less than the level of activity at the optimal permissive temperature. Typically, a non-permissive temperature is the normal body temperature of a subject. Depending on the temperature-sensitive agent, a non-permissive temperature may also be a temperature that is higher or lower than the normal body temperature of a subject.

Temperature-Sensitive Viral Vectors

In certain embodiments, a temperature-sensitive therapeutic agent of the present disclosure may comprise a temperature-sensitive viral vector. In some embodiments, an activity of the temperature-sensitive viral vector induced at a permissive temperature may include replication of the vector. As used herein, the term “temperature-sensitive viral vector” refers to any viral vector that has different levels of functionality at different temperatures. Exemplary temperature-sensitive viral vectors include, without limitation, Sendai virus vectors, Adeno associated virus vectors, retrovirus vectors, or alphavirus vectors. Exemplary temperature-sensitive alphavirus vectors include, without limitation, Venezuelan Equine Encephalitis virus vectors, Sindbis virus vectors, and Semliki Forrest virus vectors.

In some embodiments of the present disclosure, a temperature-sensitive viral vector comprises a heterologous nucleic acid (e.g., foreign nucleic acid in relation to the viral vector) comprising a coding region of a protein of interest. The heterologous nucleic acid may comprise one or more additional genetic element(s), such as a promoter in operable combination with the coding region. In preferred embodiments, the protein of interest is ZSCAN4, such as human ZSCAN4 or a variant thereof.

The permissive temperature for the temperature-sensitive viral vectors of the present disclosure typically ranges from 30° C. to 36° C. or from 38° C. to 50° C. In some embodiments, the permissive temperature is from about 31° C. to about 35° C., or 32° C. to 34° C. (33° C.±1.0° C.). In some preferred embodiments, the permissive temperature is 33° C.±0.5° C. It follows that in some embodiments, the non-permissive temperature for the temperature-sensitive viral vectors of the present disclosure is above 36° C. and below 38° C. In some preferred embodiments, the non-permissive temperature is 37° C.±0.5° C.

As disclosed herein, cells may be maintained at a permissive temperature for a period of time sufficient for the temperature-sensitive agent to induce an effect. In some embodiments, a temperature-sensitive viral vector comprises a genetic element, and an effect includes increased expression of the genetic element, wherein expression of the genetic element results in production of an RNA or polypeptide that creates a biological effect in the cells. In some preferred embodiments, the effect is a therapeutic effect.

Temperature-Sensitive Self-Replicating RNAs

In certain embodiments, a temperature-sensitive therapeutic agent of the present disclosure may comprise a temperature-sensitive self-replicating RNA. As used herein, the term “temperature-sensitive self-replicating RNA” refers to any self-replicating RNA that has different levels of functionality at different temperatures.

In some embodiments, temperature-sensitive self-replicating RNAs are created by engineering self-replicating RNAs, which are single-stranded RNAs that are usually made from the Alphavirus such as Venezuelan Equine Encephalitis Virus (VEEV), Sindbis Virus (SINV), and Semliki Forest Virus (SFV), by removing DNAs encoding structural proteins that are required for virus particle formation (Petrakova et al., 2005). In some embodiments, self-replicating RNAs encode nonstructural proteins (nsPs), which function as an RNA-dependent RNA polymerase to replicate the self-replicating RNA itself and to produce a transcript for translation. In some embodiments, self-replicating RNAs also comprise a gene of interest (GOI) comprising a coding region of a protein of interest. The gene of interest may comprise one or more additional genetic element(s), such as a promoter in operable combination with the coding region. In preferred embodiments, the protein of interest is ZSCAN4, such as human ZSCAN4 or a variant thereof. Without wishing to be bound by theory, in some embodiments, due to its positive feedback production of RNAs, self-replicating RNAs can express the GOI at a high level. In some embodiments, a temperature-sensitive self-replicating RNA may be created by mutating genes encoding nsPs.

In some embodiments, temperature-sensitive self-replicating RNAs can be delivered to mammalian cells as a naked RNA (i.e., a synthetic RNA). In some embodiments, temperature-sensitive self-replicating RNAs can be delivered to mammalian cells as a naked RNA (i.e., a synthetic RNA) encapsulated by nanoparticles. In some embodiments, nanoparticles are engineered to target specific cell types, tissues, organs, cancers, tumors, or abnormal cells. In some embodiments, temperature-sensitive self-replicating RNAs can be delivered to mammalian cells as a virus particle, which can be generated by supplementing the missing virus structural proteins by packaging helper cells. In some embodiments, virus particles are engineered to target specific cell types, tissues, organs, cancers, tumors, or abnormal cells.

When the temperature-sensitive agent is a temperature-sensitive self-replicating RNA, an activity of the temperature-sensitive self-replicating RNA induced at a permissive temperature may include replication of the RNA.

In some aspects, the permissive temperature for temperature-sensitive self-replicating RNAs of the present disclosure typically ranges from 30° C. to 36° C. In some embodiments, the permissive temperature is from about 31° C. to about 35° C., or 32° C. to 34° C. (33° C.±1.0° C.). In some preferred embodiments, the permissive temperature is 33° C.±0.5° C. 33° C.±0.5° C. It follows that in some embodiments, the non-permissive temperature for the temperature-sensitive self-replicating RNAs of the present disclosure is above 36° C. In some preferred embodiments, the non-permissive temperature is 37° C.±0.5° C.

In other aspects, the permissive temperature for temperature-sensitive self-replicating RNAs of the present disclosure typically ranges from 38° C. to 50° C. It follows that in some embodiments, the non-permissive temperature for the temperature-sensitive self-replicating RNAs of the present disclosure is above 36° C. and below 38° C. In some preferred embodiments, the non-permissive temperature is 37° C.±0.5° C.

Temperature-Sensitive Polypeptides

In certain embodiments, a temperature-sensitive therapeutic agent of the present disclosure may comprise a temperature-sensitive polypeptide. As used herein, the term “temperature-sensitive polypeptide” refers to any temperature-sensitive polypeptide that has different levels of functionality at different temperatures. In some embodiments, the temperature-sensitive polypeptide may be a temperature-sensitive ZSCAN4. In other embodiments, the temperature-sensitive polypeptide is selected from but not limited to a transcription factor for the ZSCAN4 gene.

When the temperature-sensitive agent is a temperature-sensitive polypeptide, an activity of the temperature-sensitive protein induced at a permissive temperature may include a conformational change (e.g., change to the structure or shape) of the protein.

The permissive temperature for the temperature-sensitive polypeptides of the present disclosure typically ranges from 30° C. to 36° C. or from 38° C. to 50° C. In some embodiments, the permissive temperature is from about 31° C. to about 35° C., or from 32° C. to 34° C. (33° C.±1.0° C.). In some preferred embodiments, the permissive temperature is 33° C.±0.5° C. It follows that in some embodiments, the non-permissive temperature for the temperature-sensitive self-replicating polypeptides of the present disclosure is above 36° C. and below 38° C. In some preferred embodiments, the non-permissive temperature is 37° C.±0.5° C.

Various aspects of the present disclosure relate to substantially purified polypeptides. A substantially purified polypeptide may refer to a polypeptide which is substantially free of other polypeptides, lipids, carbohydrates or other materials with which it is naturally associated. In one embodiment, the polypeptide is at least 50%, for example at least 80% free of other polypeptides, lipids, carbohydrates or other materials with which it is naturally associated. In another embodiment, the polypeptide is at least 90% free of other polypeptides, lipids, carbohydrates or other materials with which it is naturally associated. In yet another embodiment, the polypeptide is at least 95% free of other polypeptides, lipids, carbohydrates or other materials with which it is naturally associated.

Nucleic Acids and Polypeptides

Certain aspects of the present disclosure relate to transiently inducing an activity of a temperature-sensitive therapeutic agent in one or more cells, wherein the activity leads to increased expression of a nucleic acid molecule. In some embodiments, the nucleic acid is a polynucleotide. A polynucleotide may refer to a nucleic acid sequence (such as a linear sequence) of any length. Therefore, a polynucleotide includes oligonucleotides, and also gene sequences found in chromosomes. An oligonucleotide is a plurality of joined nucleotides joined by native phosphodiester bonds. An oligonucleotide is a polynucleotide of between 6 and 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid molecules.

In certain embodiments, the nucleic acid molecules or polynucleotides encode a genetic element. These polynucleotides include DNA, cDNA and RNA sequences, such as mRNA sequences, which encode a gene of interest. A coding sequence may be operably linked to a heterologous promoter to direct transcription of the genetic element. A promoter may refer to nucleic acid control sequences which direct transcription of a nucleic acid. A promoter includes necessary nucleic acid sequences near the start site of transcription. A promoter also optionally includes distal enhancer or repressor elements. A constitutive promoter is a promoter that is continuously active and is not subject to regulation by external signals or molecules. In contrast, the activity of an inducible promoter is regulated by an external signal or molecule (for example, a transcription factor). A first nucleic acid sequence is operably linked to a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and where necessary to join two protein coding regions, in the same reading frame. A heterologous polypeptide or polynucleotide refers to a polypeptide or polynucleotide derived from a different source or species. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. In one example, the promoter is a constitutive promoter, such as the CAG-promoter (Niwa et al., Gene 108(2):193-9, 1991), or the phosphoglycerate kinase (PGK)-promoter. In some embodiments, the promoter is an inducible promoter such as a tetracycline-inducible promoter (Masui et al., Nucleic Acids Res. 33:e43, 2005). Other exemplary promoters that can be used to drive expression of a genetic element include but are not limited to: lac system, the trp system, the tac system, the trc system, major operator and promoter regions of phage lambda, the control region of fd coat protein, the early and late promoters of SV40, promoters derived from polyoma, adenovirus, retrovirus, baculovirus and simian virus, the promoter for 3-phosphoglycerate kinase, the promoters of yeast acid phosphatase, and the promoter of the yeast alpha-mating factors. Genetic elements of the present disclosure may be under the control of a constitutive promoter, an inducible promoter, or any other suitable promoter described herein or other suitable promoter that will be readily recognized by one skilled in the art.

In some embodiments, inducing an activity of a temperature-sensitive agent leads to increased expression of a nucleic acid or a polypeptide, which may include increased expression by at least 1.5 fold, at least 1.6 fold, at least 1.7 fold, at least 1.8 fold, at least 1.9 fold, at least 2.0 fold, at least 2.1 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, at least 10 fold, at least 50 fold, at least 100 fold, at least 200 fold, at least 300 fold, at least 400 fold, at least 500 fold, at least 600 fold, at least 700 fold, at least 800 fold, at least 900 fold, at least 1,000 fold, at least 2,000 fold, at least 3,000 fold, at least 4,000 fold, at least 5,000 fold, at least 6,000 fold, at least 7,000 fold, at least 8,000 fold, at least 9,000 fold, at least 10,000 fold, at least 25,000 fold, at least 50,000 fold, at least 75,000 fold, at least 100,000 fold, at least 125,000 fold, at least 150,000 fold, at least 175,000 fold, at least 200,000 fold, at least 225,000 fold, at least 250,000 fold, at least 275,000 fold, at least 300,000 fold, at least 325,000 fold, at least 350,000 fold, at least 375,000 fold, at least 400,000 fold, at least 425,000 fold, at least 450,000 fold, at least 475,000 fold, at least 500,000 fold, at least 750,000 fold, or at least 1,000,000 fold, for example, relative to the polynucleotide or polypeptide expression in a human cell that has not been contacted with the agent.

Various aspects of the present disclosure relate to isolated entities, such as isolated nucleic acids or synthetic mRNA molecules. An isolated nucleic acid has been substantially separated or purified away from other nucleic acid sequences and from the cell of the organism in which the nucleic acid naturally occurs, i.e., other chromosomal and extrachromosomal DNA and RNA. The term “isolated” thus encompasses nucleic acids purified by standard nucleic acid purification methods. The term also embraces nucleic acids prepared by recombinant expression in a host cell as well as chemically synthesized nucleic acids. Similarly, isolated polypeptides have been substantially separated or purified from other polypeptides of the cells of an organism in which the protein naturally occurs, and encompasses polypeptides prepared by recombination expression in a host cell as well as chemically synthesized polypeptides. Similarly, isolated cells have been substantially separated away from other cell types.

Methods of Introducing Temperature-Sensitive Agents into Cells

In some embodiments the one or more cells are contacted with a temperature-sensitive agent. Contacting may refer to placement in direct physical association, including both in solid and liquid form. “Contacting” may be used interchangeably with “exposed.” In some cases, “contacting” includes transfecting, such as transfecting a nucleic acid molecule into a cell. In some cases “contacting” includes introducing the temperature-sensitive agent into one or more cells.

In some embodiments, temperature-sensitive agents are polynucleotides (e.g. self-replicating RNAs), and polynucleotides are introduced into cells. Introducing a nucleic acid molecule or a protein into a cell encompasses any means of delivering the nucleic acid molecule or protein into the cell. For example, nucleic acid molecules can be transfected, transduced or electroporated into a cell. In some embodiments temperature-sensitive agents are polypeptides (e.g. temperature-sensitive polypeptides), and polypeptides are introduced into cells. Delivery of polypeptides into cells can be achieved, for example, by fusing the protein to a cell-penetrating peptide, such as a peptide with a protein transduction domain (e.g., HIV-1 Tat), or a poly-arginine peptide tag (Fuchs and Raines, Protein Science 14:1538-1544, 2005). Protein transduction domains may refer to small cationic peptides that facilitate entry of larger molecules (proteins, nucleic acid molecules etc.) into a cell by a mechanism that is independent of classical endocytosis. A poly-arginine peptide tag may refer to a short peptide (generally 7 to 11 residues) comprised of arginine residues that facilitates delivery of larger molecules (such as proteins and nucleic acid molecules) into cells (see, for example, Fuchs and Raines, Protein Science 14:1538-1544, 2005).

Introduction of nucleic acids into cells with a temperature-sensitive agent may involve using a temperature-sensitive viral vector (such as integrating or non-integrating viral vectors) or a temperature-sensitive plasmid vector. Each of these methods has been described in the art and is therefore within the capabilities of one of skill in the art. A brief summary of each method that can be used to deliver a nucleic acid molecule to one or more host cells (e.g., preferably mammalian host cells such as human host cells) is provided herein. A vector may refer to a nucleic acid molecule as introduced into a host cell, thereby producing a transformed host cell. A vector may include nucleic acid sequences that permit it to replicate in a host cell, such as an origin of replication (DNA sequences that participate in initiating DNA synthesis). For example, an expression vector contains the necessary regulatory sequences to allow transcription and translation of inserted gene or genes. A vector may also include one or more selectable marker genes and other genetic elements known in the art. Vectors may include, for example, virus vectors and plasmid vectors.

Permissive Temperatures

### Incubating One or More Cells at a Permissive Temperature

Certain aspects of the present disclosure relate to transiently inducing an activity of a temperature-sensitive agent in one or more cells by incubating the cells at a permissive temperature for inducing an activity of the temperature-sensitive agent. In some embodiments, the permissive temperature may be higher or lower than the standard cell culture temperature. For example, human and rodent cells are typically cultured at a temperature of about 37° C. Accordingly, in some embodiments the permissive temperature may be lower than about 36.5° C. For example, in some embodiments the cells are cultured at a permissive temperature of 36° C., 35.5° C., 35° C., 34.5° C., 34° C., 33.5° C., 33° C., 32.5° C., 32° C., 31.5° C., 31° C., 30.5° C., or 30° C. In some preferred embodiments, the permissive temperature is from 30° C. to 36° C., or from 31° C. to 35° C., or from 32° C. to 34° C., or from 32.5° C. to 33.5° C. In some embodiments, the permissive temperature is greater than or equal to (lower limit) 30° C., 31° C., 32° C., 33° C., 34° C., or 35° C., and is less than or equal to (upper limit) 36° C., 35° C., 34° C., 33° C., 32° C. or 31° C.

In other embodiments, the permissive temperature maybe higher than about 37.5° C. For example, in some embodiments the cells are cultured at a permissive temperature of 38° C., 38.5° C., 39° C., 39.5° C., 40° C., 40.5° C., 41° C., 41.5° C., 42° C., 42.5° C., 43° C., 43.5° C., 44° C., 44.5° C., 45° C., 45.5° C., 46° C., 46.5° C., 47° C., 47.5° C., 48° C., 48.5° C., 49° C., 49.5° C., or 50° C.

In some embodiments, after incubating at a permissive temperature, the one or more cells are cultured at a non-permissive temperature wherein the activity of the temperature-sensitive agent is reduced or inhibited. For example, replication of temperature-sensitive viral vectors can be inhibited, replication of temperature-sensitive self-replicating RNAs can be inhibited, and conformational changes to temperature-sensitive polypeptides can be inhibited. This temperature shifting allows the activity of the temperature-sensitive agent to be transiently induced and then inhibited. In other embodiments, the one or more cells are administered to a subject after being cultured at a permissive temperature. The one or more cells may be administered to a subject directly from culture at a permissive temperature, or may first be transferred from a permissive temperature to a non-permissive temperature in culture and then administered to a subject. In certain embodiments, the temperature-sensitive agent is subsequently degraded. For example, non-integrating temperature-sensitive viral vectors, RNAs, and polypeptides will be degraded.

### Lowering the Core Body Temperature of a Subject to a Permissive Temperature

Certain aspects of the present disclosure relate to transiently inducing an activity of a temperature-sensitive therapeutic agent in cells in a subject by lowering the subject's core body temperature to a permissive temperature for inducing the activity of the temperature-sensitive agent. In some embodiments the subject's core body temperature is lowered using a target-temperature management (TTM) procedure. A TTM procedure is designed to achieve and maintain a specific body temperature in a subject for a duration of time. Such procedures have previously been used therapeutically to reduce the negative effects resulting from various acute health issues such as heart attacks and strokes. Equipment and general methods of using them are known in the art and can be used in the methods described herein. The procedure can be carried out using a number of methods, including cooling catheters, cooling blankets, and application of ice around the body.

After lowering the subject's core body temperature to a permissive temperature, the subject's core body temperature is maintained at the permissive temperature for a time sufficient to induce an activity of the temperature-sensitive agent. The subject's core body temperature is subsequently returned to normal core body temperature, which is a non-permissive temperature wherein the activity of the temperature-sensitive agent is reduced or inhibited. In certain embodiments the temperature-sensitive agent is subsequently degraded. For example, non-integrating temperature-sensitive viral vectors, RNAs, and polypeptides will be degraded at the non-permissive temperature. As used herein, the term “body temperature” refers to “core body temperature”, unless context clearly indicates otherwise.

### Maintaining the Surface Body Temperature of a Subject at a Permissive Temperature

Certain aspects of the present disclosure relate to exploiting the natural temperature differences in regions of a subject's body. For example, the temperature at or near the surface of a human subject's body (surface body temperature) is around 31-34° C., which is lower than the core body temperature of the human subject, which is around 37° C. As used herein, the “surface” of a subject's body refers to one or more of the epidermis, dermis, hypodermis, or muscle. The “skin” of a subject's body refers to one or both of the epidermis and dermis. Thus, suitable routes of administration to the epidermis, dermis, or hypodermis of a subject's body include intradermal and subcutaneous administration. A suitable route of administration to muscle near the surface of a subject's body is intramuscular administration.

For instance, the ts-agent is directly delivered to a specific area of the skin of a subject (in the case of vaccination) or to a broader area of the skin of a subject (in the case of treatment of a skin disease). The skin temperature (about 31-34° C.) is a permissive temperature for the ts-agent, permitting the ts-agent to function. No further action is required for the long-term expression of GOI. If termination of the function of the ts-agent is need or desired, the temperature of the treated skin is increased and transiently maintained at non-permissive temperature (>37° C.) by local application of heat (e.g., heat patch or heating blanket) or by mild therapeutic hyperthermia (e.g., warm bath or hot sauna). This therapeutic procedure is safe in that the ts-agent functions only in the intended area of the body, because the core body temperature is a non-permissive temperature (about 37° C.). In some embodiments, should the surface body temperature of the subject be higher than normal, the surface body temperature is lowered to match the permissive temperature of the ts-agent.

### Maintaining the Upper Respiratory Tract Temperature of a Subject at a Permissive Temperature

Like the surface body temperature of a human subject, the temperature of the upper respiratory tract and upper trachea of a human subject is a permissive temperature for the ts-agent, permitting the ts-agent to function. That is, the temperature of the nasal cavity and upper trachea of a human subject is about 32° C., and the temperature of the subsegmental bronchi of a human subject is about 35° C. (McFadden et al., 1985). As such, ts-agents administered intranasally to cells of the upper respiratory tract (nasal cavity, pharnyx, and/or larnyx) and/or upper trachea of a human patient are functional without lowering the core body temperature of the human patient. Intranasal administration may be done by insufflation, inhalation or instillation.

Non-Permissive Temperatures

### Incubating One or More Cells at a Non-Permissive Temperature

In vitro culture of cells is usually carried out at the normal body temperature of the subject from which the cells are derived. For example, mammalian cells, such as human cells and mouse cells, are usually cultured at about 37° C. Certain aspects of the present disclosure relate to a temperature-sensitive agent that does not function (e.g., does not replicate or express genes) at the normal body temperature of the subject. Thus, the normal body temperature of the subject is a non-permissive temperature for the temperature-sensitive agent. In some preferred embodiments, the non-permissive temperature is 37° C.±0.5° C.

### Normal Core Body Temperature of a Subject

In some embodiments, a temperature-sensitive agent, cells contacted with a temperature-sensitive agent, or cells carrying a temperature-sensitive agent, are introduced into a subject body that is maintained at the normal core body temperature. Certain aspects of the present disclosure relate to a temperature-sensitive agent that does not function, e.g., replicate or express genes, at this normal body temperature (non-permissive temperature) of the organism. This feature provides a safety mechanism that prevent the undesirable action or reactivation of the temperature-sensitive agent during the life-course of the subject.

Human Cells

Certain aspects of the present disclosure relate to transiently inducing an activity of a temperature-sensitive therapeutic agent in one or more human cells, including without limitation, adult human cells. In certain embodiments, the one or more human cells are in a subject in need of treatment with the therapeutic agent.

Various human cells find use in the methods described herein. As disclosed herein, the term “human cell(s)” refers to any cell(s) found throughout the human body during and after embryonic development, such as human embryonic cells, stem cells, pluripotent cells, differentiated cells, mature cells, somatic cells, and adult cells. In some embodiments, human cells of the present disclosure are human adult cells. As disclosed herein, the term “human adult cell(s)” refers to any cell(s) found throughout the human body after embryonic development (i.e., non-embryonic cells). Human cells of the present disclosure include, without limitation, sperm cells, oocyte cells, fertilized oocytes (i.e., zygotes), embryonic cells, mature cells, differentiated cells, somatic cells, progenitor cells, embryonic stem (ES) cells, induced pluripotent stem (iPS) cells, adult stem cells, somatic stem cells, and tissue stem cells. Adult stem cells, which are also known as somatic stem cells or tissue stem cells, may refer to undifferentiated cells, found throughout the body after embryonic development, which multiply by cell division to replenish dying cells and regenerate damaged tissues. Progenitor cells may refer to oligopotent or unipotent cells that differentiate into a specific type of cell or cell lineage. Progenitor cells are similar to stem cells but are more differentiated and exhibit limited self-renewal. Exemplary adult stem cells, tissue stem cells, and/or progenitor cells may include, without limitation, hematopoietic stem cells, mesenchymal stem cells, adipose stem cells, neuronal stem cells, intestinal stem cells, skin stem cells, and germ cells (such as, sperm cells and oocytes).

Human cells may also include, without limitation, somatic cells, mature cells, and differentiated cells. Somatic cells may refer to any cell of the body, including, without limitation, germ cells, tissue stem cells, progenitor cells, induced pluripotent stem (iPS) cells, and differentiated cells. Exemplary somatic cells, mature cells, and/or differentiated cells may include, without limitation, epidermal cells, fibroblasts, lymphocytes, hepatocytes, epithelial cells, myocytes, chondrocytes, osteocytes, adipocytes, cardiomyocytes, pancreatic p cells, keratinocytes, erythrocytes, peripheral blood cells, bone marrow cells, neurocytes, astrocytes, and germ cells. Germ cells may refer to the cells that give rise to the gametes (i.e., eggs and sperm) of organisms that reproduce sexually. In certain embodiments, germ cells include, without limitation, oocytes, and sperm cells. In some embodiment, somatic cells, mature cells, and/or differentiated cells of the present disclosure also include, without limitation, preimplantation embryos.

Human cells may also include, without limitation, cells derived from cord blood, hematopoietic stem cells, CD34+ cells, mesenchymal stem cells, vascular endothelial stem cells, tissue stem cells, granulocytes, lymphocytes, T-cells, B-cells, monocytes, macrophages, dendritic cells, red blood cells, reticulocytes, and megakaryocytes. Human cells may also include, without limitation, abnormal cells of human origins, such as cancer cells, tumor cells, malignant cells, benign cells, hyperplastic cells, hypoplastic cells, and atypical cells. Human cells may also include, without limitation, diploid cells, haploid cells, tetraploid cells, polyploid cells, cells with karyotype abnormalities, cells with chromosome abnormalities, cells with mutated genes, cells with abnormal telomere lengths, cells with short telomeres, and cells with long telomeres. Human cells may also include, without limitation, cells with epigenetic abnormalities, such as cells with hypomethylated genomic regions, cells with hypermethylated genomics regions, cells with the abnormal histone modifications such as acetylation and methylation.

In some embodiments, the subjects of the present disclosure are non-human animals. Non-human animals may refer to all animals other than humans. A non-human animal includes, but is not limited to, a non-human primate, a farm animal such as swine, cattle, and poultry, a sport animal or pet such as dogs, cats, horses, hamsters, rodents, such as mice, or a zoo animal such as lions, tigers or bears. In one embodiment, the non-human animal is a mouse.

Therapeutic Uses of Temperature-Sensitive Agents

Temperature-sensitive agents of the present disclosure may be administered by any suitable method known in the art, including, without limitation, by oral administration, sublingual administration, buccal administration, topical administration, rectal administration, via inhalation, transdermal administration, subcutaneous injection, intravenous injection, intra-arterial injection, intramuscular injection, intracardiac injection, intraosseous injection, intradermal injection, intraperitoneal injection, transmucosal administration, vaginal administration, intravitreal administration, intra-articular administration, peri-articular administration, local administration, epicutaneous administration, or any combinations thereof. In some embodiments, the composition is administered by subcutaneous injection and/or intravenous injection.

In some aspects, the methods of the present disclosure involve the use of a therapeutically effective amount of a temperature-sensitive agent. A therapeutically effective amount of an agent may refer to the amount of a therapeutic agent sufficient to achieve the intended purpose. For example, a therapeutically effective amount of a temperature-sensitive agent in a human cell to treat a disease or condition is an amount sufficient to reduce the disease or condition, or one or more symptoms of the disease or condition. A therapeutically effective amount may in some examples not treat the disease or condition, or symptoms of the disease or condition 100%. However, a decrease in any known feature or symptom of the disease or condition, such as a decrease of at least 25%, at least 50%, at least 60%, at least 70%, at least 80%, at least 90%, at least 95% can be therapeutic.

The therapeutically effective amount of a given therapeutic agent will vary with factors such as the nature of the agent, the route of administration, the size and/or age of the subject to receive the therapeutic agent, and the purpose of the administration. The therapeutically effective amount in each individual case can be determined empirically without undue experimentation by a skilled artisan according to established methods in the art.

A subject may refer to living multi-cellular vertebrate organisms, a category that includes human and non-human mammals. In some embodiments, the subject is a human. Subjects that can be treated using the methods provided herein may include mammalian subjects, such as a veterinary or human subject. Subjects may include fertilized eggs, zygotes, preimplantation embryos, embryos, fetus, newborns, infants, children, and/or adults. In some embodiments, the subject to be treated is selected, such as selecting a subject that would benefit from a therapy, particularly therapy that includes administration of a temperature-sensitive agent of the present disclosure.

Pharmaceutical compositions of the present disclosure comprise a ts-agent, such as a therapeutic ts-agent, and one or more additional compounds. As used herein, the terms “pharmaceutically acceptable carrier” and “pharmaceutically acceptable vehicle” refer to the one or more additional compound(s) (i.e., compounds other than the ts-agent). Pharmaceutically acceptable carriers suitable for use in the present disclosure are conventional. In particular, compositions and formulations suitable for pharmaceutical delivery of compositions comprising a temperature-sensitive agent are as previously described (see, e.g., Gennaro, A.R. (editor) Remington's Pharmaceutical Sciences, Mack Publishing Co., Easton, Pa., 18th edition (1990); and Felton, L. A. (editor) Remington Essentials of Pharmaceutics, Pharmaceutical Press, London, United Kingdom, 1^{st} edition, (2013)).

In general, the nature of the carrier will depend on the particular mode of administration being employed. For instance, parenteral formulations usually comprise carriers such as injectable fluids that include pharmaceutically and physiologically acceptable fluids such as water, physiological saline, balanced salt solutions, aqueous dextrose, glycerol or the like. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate. In some embodiments, pharmaceutical compositions of the present disclosure comprise a ts-agent, such as a therapeutic ts-agent, and one or more additional compounds, which facilitate the incorporation of ts-agent into cells. In the case of RNA-based ts-agent, ts-agent is encapsulated in nanoparticles. In some instances, nanoparticles is lipid-based (e.g., lipofectamine).

The therapeutic dose and regimen most appropriate for patient treatment will vary with diseases or conditions to be treated, and according to the patient's weight and other parameters. An effective dosage and treatment protocol can be determined by conventional means, starting with a low dose in laboratory animals and then increasing the dosage while monitoring the effects, and systematically varying the dosage regimen. Numerous factors can be taken into consideration by a clinician when determining an optimal dosage for a given subject. Factors include the size of the patient, the age of the patient, the general condition of the patient, the particular disease being treated, the severity of the disease, the presence of other drugs in the patient, and the like. The trial dosages would be chosen after consideration of the results of animal studies and the clinical literature.

### Mobilizing Bone Marrow Cells

In some embodiments, the methods include mobilizing bone marrow cells (including, without limitation, CD34+ cells, hematopoietic stem cells, mesenchymal stem cells, endothelial stem cells) to the spleen and peripheral blood of the subject. In some embodiments, the methods include administering a therapeutically effective amount of a temperature-sensitive agent (e.g., a temperature-sensitive therapeutic agent) of the present disclosure under conditions suitable for the temperature-sensitive agent to deliver a nucleic acid to one or more bone marrow cells (including, without limitation, CD34+ cells, hematopoietic stem cells, mesenchymal stem cells, endothelial stem cells) in the spleen.

In some embodiments of the methods disclosed herein, mobilizing bone marrow cells (including, without limitation, CD34+ cells, hematopoietic stem cells, mesenchymal stem cells, endothelial stem cells) to the spleen and peripheral blood comprises administering to the subject a therapeutically effective amount of a cytokine and/or a chemotherapeutic. In some embodiments, mobilizing bone marrow cells (including, without limitation, CD34+ cells, hematopoietic stem cells, mesenchymal stem cells, endothelial stem cells) to the spleen and peripheral blood comprises administering to the subject a therapeutically effective amount of a cytokine. In some embodiments, mobilizing bone marrow cells (including, without limitation, CD34+ cells, hematopoietic stem cells, mesenchymal stem cells, endothelial stem cells) to the spleen comprises administering to the subject a therapeutically effective amount of a chemotherapeutic. In some embodiments, mobilizing bone marrow cells (including, without limitation, CD34+ cells, hematopoietic stem cells, mesenchymal stem cells, endothelial stem cells) to the spleen comprises administering to the subject a therapeutically effective amount of a cytokine and a chemotherapeutic. Cytokines and/or chemotherapeutics may be administered by any suitable method known in the art, including, without limitation, by oral administration, sublingual administration, buccal administration, topical administration, rectal administration, via inhalation, transdermal administration, subcutaneous injection, intravenous injection, intra-arterial injection, intramuscular injection, intracardiac injection, intraosseous injection, intradermal injection, intraperitoneal injection, transmucosal administration, vaginal administration, intravitreal administration, intra-articular administration, peri-articular administration, local administration, epicutaneous administration, or any combinations thereof. In some embodiments, the cytokine and/or chemokine is administered by subcutaneous injection and/or intravenous injection.

In some embodiments, the bone marrow cells (including, without limitation, CD34+ cells, hematopoietic stem cells, mesenchymal stem cells, endothelial stem cells) of the subject are mobilized at least 4 weeks before, at least 3 weeks before, at least 2 weeks before, at least 1 week before, at least 6 days before, at least 5 days before, at least 4 days before, at least 3 days before, at least 2 days before, at least 1 day before, less than 1 day before, at least 18 hours before, at least 16 hours before, at least 12 hours before, at least 8 hours before, at least 6 hour before, or at least 1 hour before administration of the composition (e.g., any nanoparticle composition as described herein). In some embodiments, the bone marrow cells (including, without limitation, CD34+ cells, hematopoietic stem cells, mesenchymal stem cells, endothelial stem cells) of the subject are mobilized for seven consecutive days, five consecutive days, four consecutive days, three consecutive days, two consecutive days, or for one day before administration of the composition. In some embodiments, the bone marrow cells (including, without limitation, CD34+ cells, hematopoietic stem cells, mesenchymal stem cells, endothelial stem cells) of the subject are mobilized concurrently with administration of the composition.

Any cytokine capable of mobilizing bone marrow cells (including, without limitation, CD34+ cells, hematopoietic stem cells, mesenchymal stem cells, endothelial stem cells) known in the art may be used, including, without limitation, granulocyte-colony stimulating factor (G-CSF), granulocyte-macrophage colony stimulating factor (GM-CSF), erythropoietin (EPO), thrombopoietin (TPO), stem cell factor (SCF), parathyroid hormone (PTH), and any combinations thereof. In some embodiments, the cytokine is G-CSF.

In some embodiments, the G-CSF is administered to the subject at a concentration of about 0.1 μg/kg to about 100 μg/kg, or about 1.0 μg/kg to about 10 μg/kg. In some embodiments, the G-CSF is administered to the subject at a concentration of about 2.5 μg/kg. In some embodiments, the G-CSF is administered to the subject at a concentration of about 10 μg/kg.

Any chemotherapeutic capable of mobilizing bone marrow cells (including, without limitation, CD34+ cells, hematopoietic stem cells, mesenchymal stem cells, endothelial stem cells) known in the art may be used, including, without limitation, plerixafor, cyclophosphamide (CY), paclitaxel, etoposide, POL6326, BKT-140, TG-0054, NOX-A12, SEW2871, BIO 5192, bortezomib, SB-251353, FG-4497, and any combinations thereof. In some embodiments, the chemotherapeutic is plerixafor.

In some embodiments, the plerixafor is administered to the subject at a concentration of about 1 μg/kg to about 1000 μg/kg, or about 75 μg/kg to about 500 μg/kg. In some embodiments, the plerixafor is administered to the subject at a concentration of about 150 μg/kg. In some embodiments, the plerixafor is administered to the subject at a concentration of about 240 μg/kg.

In some embodiments, mobilizing bone marrow cells (including, without limitation, CD34+ cells, hematopoietic stem cells, mesenchymal stem cells, endothelial stem cells) to the spleen and peripheral blood comprises administering a therapeutically effective amount of G-CSF and a therapeutically effective amount of plerixafor. In some embodiments, the G-CSF and plerixafor are co-administered to the subject. In some embodiments, the G-CSF and plerixafor are co-administered to the subject for one day, two days, three days, four days, or more. In some embodiments, the G-CSF is administered to the subject prior to the plerixafor. In some embodiments, the G-CSF is administered to the subject one day, two days, three days, four days or more prior to the plerixafor. In some embodiments, the G-CSF is administered to the subject one day, two days, three days, four days or more prior to the plerixafor, and G-CSF and plerixafor are then co-administered to the subject for one day, two days, three days, four days or more. In some embodiments, the plerixafor is administered to the subject prior to the G-CSF. In some embodiments, the plerixafor is administered to the subject one day, two days, three days, four days or more prior to the G-CSF. In some embodiments, the plerixafor is administered to the subject one day, two days, three days, four days or more prior to the G-CSF, and G-CSF and plerixafor are then co-administered to the subject for one day, two days, three days, four days or more.

In some embodiments, one or more human cells are contacted with a temperature-sensitive agent (e.g., a temperature-sensitive therapeutic agent) that delivers a nucleic acid to the one or more human cells. In some embodiments, the nucleic acid comprises a gene of interest or encodes a protein of interest.

In some aspects, the methods of the present disclosure involve the use of a therapeutically amount of a temperature-sensitive agent (e.g., a temperature-sensitive therapeutic agent) that delivers a nucleic acid to cells of a subject in vitro or in vivo. A therapeutically effective amount of an agent may refer to the amount of a therapeutic agent sufficient to achieve the intended purpose. For example, a therapeutically effective amount of a temperature-sensitive agent (e.g., a temperature-sensitive therapeutic agent) that delivers a nucleic acid to a human cell to treat a disease or condition is an amount sufficient to reduce the disease or condition, or one or more symptoms of the disease or condition. A therapeutically effective amount may in some examples not treat the disease or condition, or symptoms of the disease or condition 100%. However, a decrease in any known feature or symptom of the disease or condition, such as a decrease of at least 25%, at least 50%, at least 60%, at least 70%, at least 80%, at least 90%, or at least 95% can be therapeutic.

In another example, a therapeutically effective amount of a cytokine and/or chemokine capable of mobilizing bone marrow cells (including, without limitation, CD34+ cells, hematopoietic stem cells, mesenchymal stem cells, endothelial stem cells) in a subject is an amount sufficient to induce mobilization of one or more bone marrow cells (including, without limitation, CD34+ cells, hematopoietic stem cells, mesenchymal stem cells, endothelial stem cells) from the bone marrow into the peripheral blood.

The therapeutically effective amount of a given temperature-sensitive agent (e.g., a temperature-sensitive therapeutic agent) will vary with factors such as the nature of the agent, the route of administration, the size and/or age of the subject to receive the therapeutic agent, and the purpose of the administration. The therapeutically effective amount in each individual case can be determined empirically without undue experimentation by a skilled artisan according to established methods in the art.

A subject may refer to living multi-cellular vertebrate organisms, a category that includes human and non-human mammals. In some embodiments, the subject is a human. Subjects that can be treated using the methods provided herein may include mammalian subjects, such as a veterinary or human subject. Subjects may include a fetus, newborns, infants, children, and/or adults. In some embodiments, the subject to be treated is selected, such as selecting a subject that would benefit from a therapy.

### Treating Diseases and Disorders

Examples of diseases or disorders that can benefit from administration of a temperature-sensitive agent (e.g., a temperature-sensitive therapeutic agent) include those disorders or diseases that are associated with gene mutation(s), abnormal telomere length, and/or abnormal epigenetic modification(s). In some aspects, the disease or disorder is a telomere biology disorder. Further examples of disorders or diseases that can benefit from administration of a temperature-sensitive agent (e.g., a temperature-sensitive therapeutic agent) include but are not limited to cancer, autoimmune diseases, and diseases in which cell regeneration is beneficial, such as neurologic injuries or a neurodegenerative disorders, as well as blindness and deafness. In some aspects, the disease or disorder is a disease of blood or a blood-forming organ.

Cancers include malignant tumors that are characterized by abnormal or uncontrolled cell growth. Cancers are frequently associated with gene mutations and aberrant telomere regulation. Exemplary cancers that can benefit from treatment with a ts-agent include but are not limited to cancers of the heart (e.g., sarcoma (angiosarcoma, fibrosarcoma, rhabdomyosarcoma, liposarcoma), myxoma, rhabdomyoma, fibroma, lipoma and teratoma), lung (e.g., bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma); gastrointestinal tract (e.g., esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), genitourinary tract (e.g., kidney (adenocarcinoma, Wilms' tumor, nephroblastoma, lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma), liver (e.g., hepatoma (hepatocellular carcinoma), cholangiocarcinoma, hepatoblastoma, angiosarcoma, hepatocellular adenoma, hemangioma), bone (e.g., osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor, chordoma, osteochondroma (osteocartilaginous exostoses), benign chondroma, chondroblastoma, chondromyxofibroma, osteoid osteoma and giant cell tumors), nervous system (e.g., skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma >pinealoma!, glioblastoma multiforme, oligodendroglioma, schwannoma, retinoblastoma, congenital tumors), spinal cord (neurofibroma, meningioma, glioma, sarcoma), gynecological cancers (e.g., uterus (endometrial carcinoma), cervix (cervical carcinoma, pre-tumor cervical dysplasia), ovaries (ovarian carcinoma, serous cystadenocarcinoma, mucinous cystadenocarcinoma, endometrioid tumors, Brenner tumor, clear cell carcinoma, unclassified carcinoma, granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma, embryonal rhabdomyosarcoma, fallopian tubes (carcinoma)), hematologic cancers (e.g., blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma)), skin (e.g., malignant melanoma, basal cell carcinoma, squamous cell carcinoma, Kaposi's sarcoma, moles, dysplastic nevi, lipoma, angioma, dermatofibroma, keloids, psoriasis), and adrenal glands (e.g., neuroblastoma).

An autoimmune diseases result from an aberrant immune response, such as the production of antibodies or cytotoxic T cells specific for a self-antigen or a subject's own cells or tissues. In some instances, the autoimmune disease is restricted to certain organs (e.g., in thyroiditis) or can involve a particular tissue in different places (e.g., Goodpasture's disease). Exemplary autoimmune diseases that can benefit from treatment with a ts-agent include but are not limited to rheumatoid arthritis, juvenile oligoarthritis, collagen-induced arthritis, adjuvant-induced arthritis, Sjogren's syndrome, multiple sclerosis, experimental autoimmune encephalomyelitis, inflammatory bowel disease (for example, Crohn's disease, ulcerative colitis), autoimmune gastric atrophy, pemphigus vulgaris, psoriasis, vitiligo, type 1 diabetes, non-obese diabetes, myasthenia gravis, Grave's disease, Hashimoto's thyroiditis, sclerosing cholangitis, sclerosing sialadenitis, systemic lupus erythematosis, autoimmune thrombocytopenia purpura, Goodpasture's syndrome, Addison's disease, systemic sclerosis, polymyositis, dermatomyositis, autoimmune hemolytic anemia, and pernicious anemia.

In some embodiments, the subject is one who has suffered a neurologic injury or suffers from a neurodegenerative disorder. A neurological injury may refer to a trauma to the nervous system (such as to the brain or spinal cord or particular neurons), which adversely affects the movement and/or memory of the injured patient. For example, such patients may suffer from dysarthria (a motor speech disorder), hemiparesis or hemiplegia. Neurologic injuries can result from a trauma to the nervous system (such as to the brain or spinal cord or particular neurons), which adversely affects the movement and/or memory of the injured patient. Such traumas may be caused by an infectious agent (e.g., a bacterium or virus), a toxin, an injury due to a fall or other type of accident, or genetic disorder, or for other unknown reasons. Accordingly, in some embodiments, a temperature-sensitive agent (e.g., a temperature-sensitive therapeutic agent) of the present disclosure that temperature-sensitive may be used to treat a neurologic injury in a subject, by modulating tissue stem cells in the nervous system of a patient that has suffered a neurologic injury, where modulating tissue stem cells in the nervous system produces neurons and glial cells, thereby repairing defects in nervous system. In some embodiments, a temperature-sensitive agent encoding various neurotrophic factors such as brain-derived neurotrophic factor (BDNF), nerve growth factor (NGF), neurotrophin-3, neurotrophin-4, ciliary neurotrophic factor, glial cell line-derived neurotrophic factor (GDNF) may be used to treat such patients. In some embodiments, the patient may have suffered a neurologic injury, such as a brain or spinal cord injury resulting from an accident, or from a stroke.

A neurodegenerative disease is a condition in which cells of the brain and/or spinal cord are lost. Neurodegenerative diseases result from deterioration of neurons or their myelin sheath which over time leads to dysfunction and disabilities. Conditions that result can cause problems with movement (such as ataxia) and with memory (such as dementia). Accordingly, in some embodiments, a temperature-sensitive agent (e.g., a temperature-sensitive therapeutic agent) of the present disclosure may be used to treat a neurodegenerative disease in a subject, by modulating tissue stem cells in the nervous system of a patient suffering from a neurodegenerative disease, where modulating tissue stem cells in the nervous system produces neurons and glial cells, thereby repairing defects in nervous system. In some embodiments, the agent modulates the nervous system of the subject and revert the degenerative conditions of the disease. Exemplary neurodegenerative diseases include but are not limited to: adrenoleukodystrophy (ALD), alcoholism, Alexander's disease, Alper's disease, Alzheimer's disease, amyotrophic lateral sclerosis (Lou Gehrig's Disease), ataxia telangiectasia, Batten disease (also known as Spielmeyer-Vogt-Sjogren-Batten disease), bovine spongiform encephalopathy (BSE), Canavan disease, cerebral palsy, Cockayne syndrome, Corticobasal degeneration, Creutzfeldt-Jakob disease, familial fatal insomnia, frontotemporal lobar degeneration, Huntington's disease, HIV-associated dementia, Kennedy's disease, Krabbe's disease, Lewy body dementia, neuroborreliosis, Machado-Joseph disease (Spinocerebellar ataxia type 3), Multiple System Atrophy, multiple sclerosis, narcolepsy, Niemann Pick disease, Parkinson's disease, Pelizaeus-Merzbacher Disease, Pick's disease, primary lateral sclerosis, prion diseases, progressive supranuclear palsy, Refsum's disease, Sandhoff disease, Schilder's disease, subacute combined degeneration of spinal cord secondary to Pernicious Anaemia, Spielmeyer-Vogt-Sjogren-Batten disease (also known as Batten disease), spinocerebellar ataxia, spinal muscular atrophy, Steele-Richardson-Olszewski disease, Tabes dorsalis, toxic encephalopathy.

Accordingly, a temperature-sensitive agent (e.g., a temperature-sensitive therapeutic agent) is administered to a subject so as to reduce or ameliorate symptoms associated with a particular disorder. Therapeutic endpoints for the treatment of cancer can include a reduction in the size or volume of a tumor, reduction in angiogenesis to the tumor, or reduction in metastasis of the tumor. If the tumor has been removed, another therapeutic endpoint can be regeneration of the tissue or organ removed. Effectiveness of cancer treatment can be measured using methods in the art, for example imaging of the tumor or detecting tumor markers or other indicators of the presence of the cancer. Therapeutic endpoints for the treatment of autoimmune diseases can include a reduction in the autoimmune response. Effectiveness of autoimmune disease treatment can be measured using methods in the art, for example measuring of autoimmune antibodies, wherein a reduction in such antibodies in the treated subject indicates that the therapy is successful. Therapeutic endpoints for the treatment of neurodegenerative disorders can include a reduction in neurodegenerative-related deficits, e.g., an increase in motor, memory or behavioral deficits. Effectiveness of treating neurodegenerative disorders can be measured using methods in the art, for example by measuring cognitive impairment, wherein a reduction in such impairment in the treated subject indicates that the therapy is successful. Therapeutic endpoints for the treatment of neurologic injuries can include a reduction in injury-related deficits, e.g., an increase in motor, memory or behavioral deficits. Effectiveness of treating neurologic injuries can be measured using methods in the art, for example by measuring mobility and flexibility, wherein an increase in such in the treated subject indicates that the therapy is successful. Treatment does not require 100% effectiveness. A reduction in the disease (or symptoms thereof) of at least about 10%, about 15%, about 25%, about 40%, about 50%, or greater, for example relative to the absence of treatment with the agent in human cells, is considered effective.

Temperature-sensitive agents (e.g., a temperature-sensitive therapeutic agents) of the present disclosure may also be used to treat atherosclerosis and/or a coronary heart disease in a subject in need thereof, by, for example, administering a temperature-sensitive agent (e.g., a temperature-sensitive therapeutic agent) of the present disclosure to the bloodstream of the subject such that the agent introduces/contacts and increases quality of vascular endothelial cells, thereby treating atherosclerosis and/or a coronary heart disease in the subject.

Temperature-sensitive agents (e.g., a temperature-sensitive therapeutic agents) of the present disclosure may also be used to provide resistance to one or more genotoxic agents in one or more human cells and/or a subject in need thereof.

Treating Diseases and Disorders Through Increased ZSCAN4 Expression

As disclosed herein, expression of ZSCAN4 increases telomere length, increases genome stability, corrects genome and/or chromosome abnormalities, protects cells against DNA damage, and/or enhances DNA repair. DNA repair may refer to a collection of processes by which a cell identifies and corrects damage to the DNA molecules of its genome. Thus, provided herein are methods related to transiently increasing the expression of ZSCAN4 in, for example, human cells to increase telomere length, increase genome stability, correct genome and/or chromosome abnormalities, protect cells against DNA damage, and/or enhance DNA repair in such cells. In some aspects, the present disclosure provides methods related to transiently increasing the expression of ZSCAN4 in, for example, human cells to increase telomere length so as to treat a disease of blood or a blood-forming organ. In some embodiments, the disease comprises bone marrow failure.

A mammalian cell (e.g., human bone marrow cell) in which a ts-agent that increases the expression of ZSCAN4 has been introduced is referred to herein as a “ZSCAN4* cell”. “ZSCAN4* cells” include, without limitation, cells that transiently express ZSCAN4. That is, ZSCAN4* cells do not necessarily continue to have measurable ZSCAN4 or to continually express ZSCAN4 mRNA or protein. In some embodiments, the action of ZSCAN4 is rapid and requires only transient expression of ZSCAN4 (e.g., on the order of hours to days). In the case of telomeres, once telomeres are extended by ZSCAN4 action, further ZSCAN4 expression is not required for a long time since telomeres only gradually shorten. Accordingly, “ZSCAN4* cells” include both cells containing the ts-agent that increases expression of ZSCAN4, and cells in which the ts-agent was introduced, but is no longer present.

Methods and compositions are provided for treating a subject in need thereof, such as a subject having a telomere abnormality. A telomere abnormality refers to any change in a telomere, such as telomere shortening, disruption of telomeric DNA repeats, or telomere DNA mutation, that disrupts one or more telomere functions. Exemplary diseases or disorders associated with a telomere abnormality in which increasing ZSCAN4 expression may be beneficial include, without limitation, diseases of telomere shortening, bone marrow failure syndromes, age-related telomere shortening diseases, and premature aging disorders.

A telomere shortening disorder (encompassed by the term “telomere biology disorder”) that may benefit from a temperature-sensitive agent that increases ZSCAN4 expression in human cells includes, without limitation, dyskeratosis congenita, Hoyeraal-Hreidarsson syndrome, Revesz syndrome, Coats plus syndrome, and idiopathic pulmonary fibrosis. In some embodiments, the telomere shortening disorder is dyskeratosis congenital.

A bone marrow failure syndrome that may benefit from a temperature-sensitive agent that increases ZSCAN4 expression in human cells includes, without limitation, Fanconi anemia, amegakaryocytic thrombocytopenia, aplastic anemia, Diamond Blackfan anemia, paroxysmal nocturnal hemoglobinuria, Pearson syndrome, Shwachman Diamond syndrome, and myelodysplastic syndrome. In some embodiments, the bone marrow failure syndrome is Fanconi anemia. In some embodiments, the subject in need of treatment suffers from both a telomere biology disorder and bone marrow failure (e.g., dyskeratosis congenita).

An age-related telomere shortening disease or a premature aging disease that may benefit from a temperature-sensitive agent that increases ZSCAN4 expression in human cells includes, without limitation, Werner syndrome, Bloom's syndrome, Hutchinson-Gilford progeria syndrome, Cockayne syndrome, Xeroderma pigmentosa, Ataxia telangiectasia, Rothmund Thomson syndrome, Trichothiodystrophy, Juberg-Marsidi syndrome, and Down syndrome.

Methods and compositions are provided for treating a subject in need thereof, such as a subject having a chromosomal abnormality. A chromosomal abnormality refers to any anomaly, aberration, or mutation in a chromosome that results in a missing, extra, or irregular portion of chromosomal DNA. In certain embodiments, the chromosome abnormality results in an atypical number of chromosomes or to a structural abnormality in one or more chromosomes. As used herein, aneuploidy may refer to an abnormal number of whole chromosomes or parts of a chromosome. An aneuploidy that may benefit from a temperature-sensitive agent that increases ZSCAN4 expression in human cells includes, without limitation, a chromosome nullisomy, a chromosome monosomy, a chromosome trisomy, a chromosome tetrasomy, and a chromosome pentasomy. Examples of human aneuploidies include, without limitation, trisomy 21, trisomy 16, trisomy 18 (Edwards syndrome), trisomy 13 (Patau syndrome), monosomy X (Turner's syndrome), XXX aneuploidy, XXY aneuploidy, and XYY aneuploidy. Examples of human segmental aneuploidies include, without limitation, 1p36 duplication, dup(17)(p11.2p11.2) syndrome, Pelizaeus-Merzbacher disease, dup(22)(q11.2q11.2) syndrome, and cat-eye syndrome. In some embodiments, the aneuploidy includes one or more deletions of sex or autosomal chromosomes, which can result in a condition such as Cri-du-chat syndrome, Wolf-Hirschhorn, Williams-Beuren syndrome, Charcot-Marie-Tooth disease, Hereditary neuropathy with liability to pressure palsies, Smith-Magenis syndrome, Neurofibromatosis, Alagille syndrome, Velocardiofacial syndrome, DiGeorge syndrome, Steroid sulfatase deficiency, Kallmann syndrome, Microphthalmia with linear skin defects, Adrenal hypoplasia, Glycerol kinase deficiency, Pelizaeus-Merzbacher disease, Testis-determining factor on Y, Azoospermia (factor a), Azoospermia (factor b), Azoospermia (factor c), or 1p36 deletion.

### ZSCAN4 Polynucleotides

In some embodiments, a therapeutic temperature-sensitive agent of the present disclosure that increases expression of ZSCAN4 is a nucleic acid molecule including a nucleic acid sequence (coding region) encoding a ZSCAN4 protein. Nucleic acid molecules include DNA, cDNA and RNA (mRNA) molecules encoding a ZSCAN4 protein. It is understood that all polynucleotides encoding a ZSCAN4 protein are included herein, as long as they encode a ZSCAN4 protein, variant, or fragment thereof with a ZSCAN4 activity, such as the ability to modulate genome stability or telomere length. Genome stability refers to the ability of a cell to faithfully replicate DNA and maintain integrity of its DNA replication machinery. Long telomeres are thought to provide a buffer against cellular senescence and be generally indicative of genome stability and overall cell health. Chromosome stability (e.g., few mutations, no chromosomal rearrangements or change in number) is also associated with genome stability. A loss of genome stability is associated with cancer, neurological disorders and premature aging. Signs of genome instability include elevated mutation rates, gross chromosomal rearrangements, alterations in chromosome number, and shortening of telomeres.

The sequences of ZSCAN4 nucleic acid molecules are known in the art. ZSCAN4 nucleic acid sequences include, without limitation, sequences encoding a mouse Zscan4 protein that exhibits 2-cell embryonic stage- or ES cell-specific expression (including Zscan4a, Zscan4b, Zscan4c, Zscan4d, Zscan4e and Zscan4f) or an ortholog thereof. In some embodiments, the ortholog is human ZSCAN4. Nucleic acid sequences encoding human ZSCAN4 and orthologs thereof are disclosed in the sequence listing of U.S. Pat. No. 10,335,456 B1 to Ko, and are incorporated herein by reference.

Fragments and variants of ZSCAN4 polynucleotides can be prepared by one of skill in the art using standard molecular techniques. In some embodiments, the ZSCAN4 polynucleotide encodes a truncated form of the ZSCAN4 protein lacking one or more zinc finger domains of the naturally occurring ZSCAN4 protein. In some embodiments, the ZSCAN4 polynucleotide encodes a variant of the ZSCAN4 protein. The nucleotides can be ribonucleotides, deoxyribonucleotides, or modified forms of either nucleotide. The term includes single- and double-stranded forms of DNA. A recombinant nucleic acid is one that has a sequence that is not naturally occurring or has a sequence that is made by an artificial combination of two otherwise separated segments of sequence.

A ZSCAN4 coding region may be operably linked to a promoter to direct transcription of the coding region. A promoter refers to a nucleic acid control sequence that directs transcription of an operably linked coding region. A promoter includes necessary nucleic acid sequences near the start site of transcription. A promoter also optionally includes distal enhancer or repressor elements. A constitutive promoter is a promoter that is continuously active and is not subject to regulation by external signals or molecules. In contrast, the activity of an inducible promoter is regulated by an external signal or molecule (for example, a transcription factor). A first nucleic acid sequence is operably linked to a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and where necessary to join two protein coding regions, in the same reading frame. A heterologous polypeptide or polynucleotide refers to a polypeptide or polynucleotide derived from a different source or species. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. In one example, the promoter is a constitutive promoter, such as the CAG-promoter (Niwa et al., Gene 108(2):193-9, 1991. In some embodiments, the promoter is an inducible promoter, such as a tetracycline-inducible promoter (Masui et al., Nucleic Acids Res. 33:e43, 2005). Other exemplary promoters that can be used to drive Zscan4 expression include but are not limited to: lac system, the trp system, the tac system, the trc system, major operator and promoter regions of phage lambda, the control region of fd coat protein, the early and late promoters of SV40, promoters derived from polyoma, adenovirus, retrovirus, baculovirus and simian virus, the promoter for 3-phosphoglycerate kinase, the promoters of yeast acid phosphatase, and the promoter of the yeast alpha-mating factors. In some embodiments, a native ZSCAN4 promoter is used.

### ZSCAN4 Polypeptides

It is understood that all ZSCAN4 polypeptides are included herein, as long as they retain a ZSCAN4 activity, such as the ability to modulate genome stability or telomere length. The terms “polypeptide” and “protein” are used interchangeably herein, and include naturally occurring ZSCAN4 proteins, variants, or fragments thereof with a ZSCAN4 activity.

The amino acid sequences of ZSCAN4 proteins are known in the art. ZSCAN4 amino acid sequences include, without limitation, sequences of a mouse Zscan4 protein that exhibits 2-cell embryonic stage- or ES cell-specific expression (including Zscan4a, Zscan4b, Zscan4c, Zscan4d, Zscan4e and Zscan4f) or an ortholog thereof. In some embodiments, the ortholog is human ZSCAN4. Amino acid sequences encoding human ZSCAN4 and orthologs thereof are disclosed in the sequence listing of U.S. Pat. No. 10,335,456 B1 to Ko, and are incorporated herein by reference.

Fragments and variants of ZSCAN4 proteins can be prepared by one of skill in the art using standard molecular techniques. In some embodiments, the ZSCAN4 protein is a truncated form of ZSCAN4 lacking one or more zinc finger domains of the naturally occurring ZSCAN4 protein. In some embodiments, the ZSCAN4 protein is a variant of the naturally occurring ZSCAN4 protein.

In some preferred embodiments, the amino acid sequence of the human ZSCAN4 protein comprises SEQ ID NO:38, or one of the group consisting of SEQ ID NOs:39-42.

```
hZSCAN4 (aa1-433:):
(SEQ ID NO: 38)
MALDLRTIFQCEPSENNLGSENSAFQQSQGPAVQREEGISEFSRMVLNSFQDSNNSYARQ
ELQRLYRIFHSWLQPEKHSKDEIISLLVLEQFMIGGHCNDKASVKEKWKSSGKNLERFIE
DLTDDSINPPALVHVHMQGQEALFSEDMPLRDVIVHLTKQVNAQTTREANMGTPSQTS
QDTSLETGQGYEDEQDGWNSSSKTTRVNENITNQGNQIVSLIIIQEENGPRPEEGGVSSD
NPYNSKRAELVTARSQEGSINGITFQGVPMVMGAGCISQPEQSSPESALTHQSNEGNSTC
EVHQKGSHGVQKSYKCEECPKVFKYLCHLLAHQRRHRNERPFVCPECQKGFFQISDLR
VHQIIHTGKKPFTCSMCKKSFSHKTNLRSHERIHTGEKPYTCPFCKTSYRQSSTYHRHMR
THEKITLPSVPSTPEAS.
hZSCAN4 (aa1-311):
  (SEQ ID NO: 39)
MALDLRTIFQCEPSENNLGSENSAFQQSQGPAVQREEGISEFSRMVLNSFQDSNNSYARQ
ELQRLYRIFHSWLQPEKHSKDEIISLLVLEQFMIGGHCNDKASVKEKWKSSGKNLERFIE
DLTDDSINPPALVHVHMQGQEALFSEDMPLRDVIVHLTKQVNAQTTREANMGTPSQTS
QDTSLETGQGYEDEQDGWNSSSKTTRVNENITNQGNQIVSLIIIQEENGPRPEEGGVSSD
NPYNSKRAELVTARSQEGSINGITFQGVPMVMGAGCISQPEQSSPESALTHQSNEGNSTC
EVHQKGSHGVQKS.
hZSCAN4 (aa1-339):
  (SEQ ID NO: 40)
MALDLRTIFQCEPSENNLGSENSAFQQSQGPAVQREEGISEFSRMVLNSFQDSNNSYARQ
ELQRLYRIFHSWLQPEKHSKDEIISLLVLEQFMIGGHCNDKASVKEKWKSSGKNLERFIE
DLTDDSINPPALVHVHMQGQEALFSEDMPLRDVIVHLTKQVNAQTTREANMGTPSQTS
QDTSLETGQGYEDEQDGWNSSSKTTRVNENITNQGNQIVSLIIIQEENGPRPEEGGVSSD
NPYNSKRAELVTARSQEGSINGITFQGVPMVMGAGCISQPEQSSPESALTHQSNEGNSTC
EVHQKGSHGVQKSYKCEECPKVFKYLCHLLAHQRRHRNERP.
hZSCAN4 (aa1-367):
  (SEQ ID NO: 41)
MALDLRTIFQCEPSENNLGSENSAFQQSQGPAVQREEGISEFSRMVLNSFQDSNNSYARQ
ELQRLYRIFHSWLQPEKHSKDEIISLLVLEQFMIGGHCNDKASVKEKWKSSGKNLERFIE
DLTDDSINPPALVHVHMQGQEALFSEDMPLRDVIVHLTKQVNAQTTREANMGTPSQTS
QDTSLETGQGYEDEQDGWNSSSKTTRVNENITNQGNQIVSLIIIQEENGPRPEEGGVSSD
NPYNSKRAELVTARSQEGSINGITFQGVPMVMGAGCISQPEQSSPESALTHQSNEGNSTC
EVHQKGSHGVQKSYKCEECPKVFKYLCHLLAHQRRHRNERPFVCPECQKGFFQISDLR
VHQIIHTGKKP.
hZSCAN4 (aa1-395):
(SEQ ID NO: 42)
MALDLRTIFQCEPSENNLGSENSAFQQSQGPAVQREEGISEFSRMVLNSFQDSNNSYARQ
ELQRLYRIFHSWLQPEKHSKDEIISLLVLEQFMIGGHCNDKASVKEKWKSSGKNLERFIE
DLTDDSINPPALVHVHMQGQEALFSEDMPLRDVIVHLTKQVNAQTTREANMGTPSQTS
QDTSLETGQGYEDEQDGWNSSSKTTRVNENITNQGNQIVSLIIIQEENGPRPEEGGVSSD
NPYNSKRAELVTARSQEGSINGITFQGVPMVMGAGCISQPEQSSPESALTHQSNEGNSTC
EVHQKGSHGVQKSYKCEECPKVFKYLCHLLAHQRRHRNERPFVCPECQKGFFQISDLR
VHQIIHTGKKPFTCSMCKKSFSHKTNLRSHERIHTGEKP.
```

In some embodiments, the amino acid sequence of the human ZSCAN4 protein is at least 90%, at least 91%, at least 92%, at least 93%, at least 94%, at least 95%, at least 96%, at least 97%, at least 98%, at least 99%, or 100% identical to one of the group consisting of SEQ ID NOs:38-42.

The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and monkey sequences), compared to species more distantly related (such as human and mouse sequences).

The terms “identical” or percent “identity,” in the context of two or more sequences (e.g., nucleic acid sequences or amino acid sequences), may refer to two or more sequences or subsequences that are the same. Two sequences are substantially identical if two sequences have a specified percentage of amino acid residues or nucleotides that are the same (i.e., 95%, 96%, 97%, 98%, 99% or 100% identity over a specified region, or, when not specified, over the entire sequence), when compared and aligned for maximum correspondence over a comparison window, or designated region as measured using one of the following sequence comparison algorithms or by manual alignment and visual inspection.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. When comparing two sequences for identity, it is not necessary that the sequences be contiguous, but any gap would carry with it a penalty that would reduce the overall percent identity. For blastp, the default parameters are Gap opening penalty=11 and Gap extension penalty=1. For blastn, the default parameters are Gap opening penalty=5 and Gap extension penalty=2.

A comparison window may include reference to a segment of any one of the number of contiguous positions including, but not limited to from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known. Optimal alignment of sequences for comparison can be conducted, e.g., by the local homology algorithm of Smith and Waterman (1981), by the homology alignment algorithm of Needleman and Wunsch (1970) J Mol Biol 48(3):443-453, by the search for similarity method of Pearson and Lipman (1988) Proc Natl Acad Sci USA 85(8):2444-2448, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection [see, e.g., Brent et al., (2003) Current Protocols in Molecular Biology, John Wiley & Sons, Inc. (Ringbou Ed)].

Two examples of algorithms that are suitable for determining percent sequence identity and sequence similarity are the BLAST and BLAST 2.0 algorithms, which are described in Altschul et al. (1997) Nucleic Acids Res 25(17):3389-3402 and Altschul et al. (1990) J. Mol Biol 215(3)-403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. For amino acid sequences, the BLASTP program uses as defaults a word length of 3, and expectation (E) of 10, and the BLOSUM62 scoring matrix [see Henikoff and Henikoff, (1992) Proc Natl Acad Sci USA 89(22):10915-10919] alignments (B) of 50, expectation (E) of 10, M=5, N=−4, and a comparison of both strands. For nucleotide sequences, the BLASTN program uses as defaults a word length (W) of 11, an expectation (E) or 10, M=5, N=−4, and a comparison of both strands.

The BLAST algorithm also performs a statistical analysis of the similarity between two sequences (see, e.g., Karlin and Altschul, (1993) Proc Natl Acad Sci USA 90(12):5873-5877). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

In certain embodiments, the Zscan4 polynucleotide encoding a Zscan4 polypeptide is a human ZSCAN4 polynucleotide or homolog thereof. In some embodiments, the Zscan4 polynucleotide encodes a human ZSCAN4 protein comprising the amino acid sequence that is at least 90%, at least 91%, at least 92%, at least 93%, at least 94%, at least 95%, at least 96%, at least 97%, at least 98%, at least 99%, or 100% identical to one of the group consisting of SEQ ID NOs:38-42.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control.

### EXEMPLARY EMBODIMENTS

1. A method for transiently inducing a temperature-sensitive activity of a temperature-sensitive agent, comprising:

i) incubating one or more cells comprising the temperature-sensitive agent at a permissive temperature to induce the temperature-sensitive activity for a period of time sufficient for the temperature-sensitive activity to produce an effect in the one or more cells; and

ii) incubating the one or more cells at a non-permissive temperature, wherein the non-permissive temperature reduces the temperature-sensitive activity of the temperature-sensitive agent,

wherein the temperature-sensitive agent comprises a therapeutic agent comprising a human ZSCAN4 protein, or a nucleic acid comprising a coding region of human ZSCAN4, and the effect comprises a therapeutic effect.
2. The method of embodiment 1, further comprising before step i): contacting the one or more cells with the temperature-sensitive agent.
3. The method of embodiment 2, wherein the one or more cells are at the permissive temperature when contacted with the temperature-sensitive agent.
4. The method of any one of embodiments 1-3, further comprising administering the one or more cells to a subject in need of the therapeutic effect.
5. The method of any one of embodiments 1-3, wherein incubating the one or more cells at a non-permissive temperature comprises administering the one or more cells to a subject in need of the therapeutic effect, wherein the subject's body temperature is the non-permissive temperature.
6. The method of embodiment 4 or 5, wherein the one or more cells are further incubated at the non-permissive temperature prior to administering the one or more cells to the subject.
7. The method of any one of embodiments 2-6, wherein the one or more cells were isolated from the subject before contacting the one or more cells with the temperature-sensitive agent.
8. The method of any one of embodiments 1-7, wherein the therapeutic effect comprises increasing telomere length of the one or more cells.
9. The method of any one of embodiments 1-8, wherein the one or more cells are mammalian cells.
10. The method of any one of embodiments 3-8, wherein the subject is a human subject.
11. A method for transiently inducing a temperature-sensitive activity of a temperature-sensitive agent in a human subject, wherein one or more cells of the subject comprise the temperature-sensitive agent, wherein the temperature-sensitive activity of the temperature-sensitive agent is induced at a permissive temperature, and wherein the permissive temperature is lower than the body temperature of the subject, comprising:

i) lowering the body temperature of the subject to the permissive temperature;

ii) maintaining said lowered body temperature for a period of time sufficient for the temperature-sensitive activity to induce an effect in the subject; and

iii) raising the body temperature of the subject back to normal body temperature,

wherein the temperature-sensitive agent comprises a therapeutic agent comprising a human ZSCAN4 protein, or a nucleic acid comprising a coding region of human ZSCAN4, and the effect is a therapeutic effect.
12. A method for transiently inducing a temperature-sensitive activity of a temperature-sensitive agent in a human subject, wherein the temperature-sensitive activity of the temperature-sensitive agent is induced at a permissive temperature, and wherein the permissive temperature is lower than the body temperature of the subject, comprising:

i) lowering the body temperature of the subject to the permissive temperature;

ii) administering the temperature-sensitive agent to one or more cells of the subject;

iii) maintaining said lowered body temperature for a period of time sufficient for the temperature-sensitive activity to induce an effect in the subject; and

iv) raising the body temperature of the subject back to normal body temperature,

wherein step (i) is performed before, after, or simultaneously with step (ii), wherein the temperature-sensitive agent comprises a therapeutic agent comprising a human ZSCAN4 protein, or a nucleic acid comprising a coding region of human ZSCAN4, and the effect is a therapeutic effect.
13. The method of embodiment 12, wherein the temperature-sensitive agent is administered systemically.
14. The method of embodiment 13, wherein the temperature-sensitive agent is administered intravenously.
15. The method of embodiment 12, wherein the temperature-sensitive agent is administered to a specific tissue or organ of the subject.
16. The method of embodiment 15, wherein the temperature-sensitive agent is administered to the brain and spinal cord by epidural injection.
17. The method of embodiment 15, wherein the temperature-sensitive agent is administered by percutaneous injection into a target organ.
18. The method of embodiment 15, wherein the temperature-sensitive agent is administered by endoscopy with an injection needle catheter into a target organ.
19. The method of embodiment 15, wherein the temperature-sensitive agent is administered by angiocatheter into a target organ.
20. The method of any one of embodiments 17-19, wherein the target organ is selected from the group consisting of the liver, kidneys, skeletal muscles, cardiac muscles, pancreas, spleen, heart, brain, spinal cord, skin, eye, lung, intestine, thymus, bone marrow, bone, and cartilage.
21. The method of embodiment 12, wherein the temperature-sensitive agent is administered by inhalation.
22. The method of any one of embodiments 11-21, wherein lowering the body temperature of a subject comprises using a targeted temperature management (TTM) procedure, wherein the TTM procedure comprises application to the subject of one of the group consisting of a cooling catheter, a cooling blanket, and ice.
23. The method of any one of embodiments 11-22, wherein the subject is a mammalian subject, optionally wherein the subject is human.
24. The method of any one of embodiments 1-23, wherein the temperature-sensitive agent comprises a human ZSCAN4 protein.
25. The method of any one of embodiments 1-23, wherein the temperature-sensitive agent comprises a nucleic acid comprising a coding region of human ZSCAN4.
26. The method of embodiment 25, wherein a temperature-sensitive viral vector comprises the nucleic acid comprising the coding region of human ZSCAN4.
27. The method of embodiment 26, wherein the temperature-sensitive viral vector is selected from the group consisting of a Sendai virus, an Adeno virus, an Adeno-associated virus, a Retrovirus, and an Alphavirus.
28. The method of embodiment 26, wherein said temperature-sensitive viral vector is an Alphavirus.
29. The method of embodiment 28, wherein said Alphavirus is selected from the group consisting of a Venezuelan equine encephalitis virus, a Sindbis virus, and a Semliki Forrest virus.
30. The method of embodiment 26, wherein the temperature-sensitive viral vector is a Sendai virus.
31. The method of embodiment 30, wherein the Sendai virus is SeV18+/TS15ΔF.
32. The method of any one of embodiments 26-31, wherein the temperature sensitive activity comprises replication and transcription of the temperature-sensitive viral vector.
33. The method of embodiment 25, wherein a temperature-sensitive self-replicating RNA comprises the nucleic acid comprising the coding region of human ZSCAN4.
34. The method of embodiment 33, wherein the self-replicating RNA comprises an Alphavirus replicon lacking a viral structural protein coding region.
35. The method of embodiment 34, wherein the Alphavirus is selected from the group consisting of a Venezuelan equine encephalitis virus, a Sindbis virus, and a Semliki Forrest virus.
36. The method of any one of embodiments 33-35, wherein the temperature-sensitive activity comprises one or both of replication and transcription of the temperature-sensitive self-replicating RNA.
37. The method of any one of embodiments 25-36, wherein the coding region is operably linked to a promoter.
38. The method of any one of embodiments 1-10, wherein the period of time sufficient for the temperature-sensitive activity to produce the therapeutic effect ranges from about 12 hours to about 12 weeks, optionally wherein the period of time is from 1 to 7 days.
39. The method of any one of embodiments 11-37, wherein the period of time sufficient to induce the therapeutic effect in the subject is from about 12 hours to about 7 days, optionally wherein the period of time is from about 12 hours to about 72 hours.
40. The method of any one of embodiments 1-39, wherein the permissive temperature ranges from 30° C. to 36° C. or from 38° C. to 50° C.
41. The method of embodiment 40, wherein the permissive temperature is 33° C.±0.5° C.
42. The method of embodiment 40 or embodiment 41, wherein the non-permissive temperature is 37° C.±0.5° C.
43. The method of any one of embodiments 1-42, wherein the one or more cells are human cells.
44. The method of embodiment 43, wherein the one or more human cells are adult stem cells, tissue stem cells, progenitor cells, embryonic stem cells, or induced pluripotent stem cells.
45. The method of embodiment 43, wherein the one or more human cells are selected from the group consisting of hematopoietic stem cells, mesenchymal stem cells, endothelial stem, cells adipose stem cells, neuronal stem cells, and germ stem cells.
46. The method of embodiment 43, wherein the one or more human cells are somatic cells, mature cells, or differentiated cells.
47. The method of embodiment 46, wherein the one or more human cells are selected from the group consisting of epidermal cells, fibroblasts, lymphocytes, hepatocytes, epithelial cells, myocytes, chondrocytes, osteocytes, adipocytes, cardiomyocytes, pancreatic cells, pancreatic p cells, keratinocytes, erythrocytes, peripheral blood mononuclear cells (PBMCs), neurons, glia cells, neurocytes, astrocytes, germ cells, sperm cells, and oocytes.
48. The method of embodiment 43, wherein the one or more human cells are human bone marrow cells.
49. The method of embodiment 48, wherein the human bone marrow cells are CD34+ hematopoietic stem cells.
50. The method of embodiment 48 or embodiment 49, wherein the human subject suffers from a telomere biology disorder, optionally wherein the subject suffers from bone marrow failure.
51. A method of treating a disease of blood or a blood-forming organ, comprising:

i) mobilizing hematopoietic stem cells from bone marrow to peripheral blood of a human subject suffering from the disease;

ii) isolating CD34+ cells from a sample of peripheral blood mononuclear cells obtained from the subject;

iii) incubating the isolated CD34+ cells at a temperature of 33° C.±0.5° C.;

iv) contacting the incubated CD34+ cells with a temperature-sensitive Sendai viral vector comprising a heterologous nucleic acid comprising a coding region of human ZSCAN4;

v) maintaining the contacted CD34+ cells at a permissive temperature of 33° C.±0.5° C. for a period of at least about 12-72 hours, wherein replication and transcription of the temperature-sensitive Sendai viral vector occurs at the permissive temperature leading to increased expression of human ZSCAN4; and

vi) infusing the contacted CD34+ cells into the subject under conditions suitable for engrafting the cells to treat the disease.

52. A method of treating a disease of blood or a blood-forming organ, comprising:

i) mobilizing hematopoietic stem cells from bone marrow cell to peripheral blood of a human subject suffering from the disease;

ii) isolating CD34+ cells from a sample of peripheral blood mononuclear cells obtained from the subject;

iii) contacting the isolated CD34+ cells with a temperature-sensitive Sendai viral vector comprising a heterologous nucleic acid comprising a coding region of human ZSCAN4;

iv) incubating the contacted CD34+ cells at a permissive temperature of 33° C.±0.5° C. for a period of at least about 12-72 hours, wherein replication and transcription of the temperature-sensitive Sendai viral vector occurs at the permissive temperature leading to increased expression of human ZSCAN4; and

v) infusing the contacted CD34+ cells into the subject under conditions suitable for engrafting the cells to treat the disease.

53. The method of embodiment 51, further comprising after step v) incubating the contacted CD34+ cells at a non-permissive temperature of 37° C.±0.5° C. prior to infusing the contacted CD34+ cells into the subject, wherein replication and transcription of the temperature-sensitive Sendai viral vector and expression of human ZSCAN4 ceases at the non-permissive temperature.
54. The method of embodiment 52, further comprising after step iv) incubating the contacted CD34+ cells at a non-permissive temperature of 37° C.±0.5° C. prior to infusing the contacted CD34+ cells into the subject, wherein replication and transcription of the temperature-sensitive Sendai viral vector and expression of human ZSCAN4 ceases at the non-permissive temperature.
55. The method of embodiment 53 or embodiment 54, wherein the contacted CD34+ cells are incubated at the non-permissive temperature of 37° C.±0.5° C. for about 30 minutes to about 10 days, optionally for about 30-180 minutes.
56. The method of any one of embodiments 51-55, wherein the hematopoietic stem cells are mobilized by administration of one or both of granulocyte-colony stimulation factor and plerixafor to the subject.
57. The method of any one of embodiments 51-56, wherein the peripheral blood mononuclear cells are obtained from the subject by apheresis.
58. The method of any one of embodiments 51-57, wherein the CD34+ cells are isolated from the peripheral blood mononuclear cells by positive selection using an anti-CD34 antibody and magnetic beads.
59. The method of any one of embodiments 51-58, wherein the contacted CD34+ cells are washed and resuspended in a sterile, isotonic aqueous solution prior to infusion.
60. The method of embodiment 59, wherein the contacted CD34+ cells are intravenously infused at a dose of about 1.0×10\hat{ }5 cells/kg to about 1.0×10\hat{ }7 cells/kg, optionally about 2.0-8.0×10\hat{ }6 cells/kg.
61. A method of treating a disease of blood or a blood-forming organ, comprising:

i) administering to a human subject suffering from the disease a temperature-sensitive Sendai viral vector comprising a heterologous nucleic acid comprising a coding region of human ZSCAN4;

ii) lowering the subject's core body temperature to a permissive temperature of 33° C.±0.5° C.;

iii) maintaining the subject's core body temperature at the permissive temperature for a period of from about 12 hours to about 7 days, or for about 12-72 hours, wherein replication and transcription of the temperature-sensitive Sendai viral vector occurs at the permissive temperature leading to increased expression of human ZSCAN4; and

iv) allowing the subject's core body temperature to return to a normal, non-permissive temperature of 37° C.±0.5° C., wherein replication and transcription of the temperature-sensitive Sendai viral vector and expression of human ZSCAN4 ceases at the non-permissive temperature.

62. A method of treating a disease of blood or a blood-forming organ, comprising:

i) lowering the core body temperature of a subject suffering from the disease to a permissive temperature of 33° C.±0.5° C.;

ii) administering to the subject a temperature-sensitive Sendai viral vector comprising a heterologous nucleic acid comprising a coding region of human ZSCAN4;

iii) maintaining the subject's core body temperature at the permissive temperature for a period of from about 12 hours to about 7 days, or for about 12-72 hours, wherein replication and transcription of the temperature-sensitive Sendai viral vector occurs at the permissive temperature leading to increased expression of human ZSCAN4; and

iv) allowing the subject's core body temperature to return to a normal, non-permissive temperature of 37° C.±0.5° C., wherein replication and transcription of the temperature-sensitive Sendai viral vector and expression of human ZSCAN4 ceases at the non-permissive temperature.

63. The method of embodiment 61 or embodiment 62, wherein the subject's core body temperature is lowered using a targeted temperature management (TTM) procedure, wherein the TTM procedure comprises application to the subject of one of the group consisting of a cooling catheter, a cooling blanket, and ice.
64. The method of any one of embodiments 51-63, wherein the human subject is diagnosed with bone marrow failure prior to treatment, optionally wherein the bone marrow failure comprises one or more of neutropenia, thrombocytopenia, and anemia.
65. The method of any one of embodiments 51-64, wherein the subject does not have cancer.
66. The method of any one of embodiments 51-65, wherein the disease is a telomere biology disorder.
67. The method of embodiment 66, where the telomere biology disorder is selected from the group consisting of dyskeratosis congenita, Hoyeraal-Hreidarsson syndrome, Revesz syndrome, Coats plus syndrome, idiopathic pulmonary fibrosis, and cirrhosis.
68. The method of embodiment 66, wherein the telomere biology disorder is defined by one or both of:

i) age-adjusted mean telomere length of less than 1 percentile in one or more of peripheral blood lymphocytes, B-cells, and naïve T-cells; and

ii) a pathogenic mutation in a gene selected from the group consisting of DKC1, TERC, TERT, NOP10, NHP2, TINF2, CTC1, PARN, RTEL1, ACD, USB1, and WRAP53.

69. The method of any one of embodiments 51-63, wherein the disease is a bone marrow failure syndrome.
70. The method of embodiment 69, wherein the bone marrow failure syndrome is selected from the group consisting of Fanconi anemia, amegakaryocytic thrombocytopenia, aplastic anemia, Diamond Blackfan anemia, paroxysmal nocturnal hemoglobinuria, Pearson syndrome, Shwachman Diamond syndrome, and myelodysplastic syndrome.
71. The method of embodiment 64, wherein the disease is associated with a karyotype abnormality.
72. The method of any one of embodiments 1-71, wherein the amino acid sequence of human ZSCAN4 comprises SEQ ID NO:38 or is at least 95% identical to SEQ ID NO:38.
73. The method of any one of embodiments 1-71, wherein the amino acid sequence of human ZSCAN4 comprises one of the group consisting of SEQ ID NO:39, SEQ ID NO:40, SEQ ID NO:41, and SEQ ID NO:42, or is at least 95% identical to one of the group consisting of SEQ ID NO:39, SEQ ID NO:40, SEQ ID NO:41, and SEQ ID NO:42.
74. The method of any one of embodiments 26-50, wherein the temperature-sensitive viral vector or the temperature-sensitive self-replicating RNA comprises a nonstructural protein coding region with an insertion of 12-18 nucleotides, wherein the insertion results in expression of a nonstructural Protein 2 (nsP2=helicase proteinase) comprising from 4 to 6 additional amino acids between beta sheet 5 and beta sheet 6 of the nsP2, optionally wherein the additional amino acids result in temperature-sensitivity of the viral vector or the self-replicating RNA.
75. The method of embodiment 74, wherein the additional amino acids comprise one sequence selected from the group consisting of SEQ ID NO:43 (GCGRT), SEQ ID NO:44 (TGAAA), and SEQ ID NO:45 (LRPHP).
76. The method of embodiment 74, wherein the additional amino acids comprise the sequence of SEQ ID NO:44 (TGAAA).
77. The method of embodiment 76, wherein the amino acid sequence of the NsP2 comprises one sequence selected from the group consisting of SEQ ID NOs:29-36.
78. A temperature-sensitive agent, wherein the agent is a temperature-sensitive viral vector or a temperature-sensitive self-replicating RNA comprising a heterologous nucleic acid comprising a coding region of human ZSCAN4, and a nonstructural protein coding region with an insertion of 12-18 nucleotides, wherein the insertion results in expression of a nonstructural Protein 2 (nsP2=helicase proteinase) comprising from 4 to 6 additional amino acids between beta sheet 5 and beta sheet 6 of the nsP2, optionally wherein the additional amino acids result in temperature-sensitivity of the viral vector or the self-replicating RNA.
79. The temperature-sensitive agent of embodiment 78, wherein the additional amino acids comprise one sequence selected from the group consisting of SEQ ID NO:43 (GCGRT), SEQ ID NO:44 (TGAAA), and SEQ ID NO:45 (LRPHP).
80. The temperature-sensitive agent of embodiment 78, wherein the additional amino acids comprise the sequence of SEQ ID NO:44 (TGAAA).
81. The temperature-sensitive agent of embodiment 81, wherein the amino acid sequence of the NsP2 comprises one sequence selected from the group consisting of SEQ ID NOs:29-36.
82. The temperature-sensitive agent of any one of embodiments 78-81, wherein the agent is a temperature-sensitive Alphavirus vector.
83. The temperature-sensitive agent of any one of embodiments 78-81, wherein the agent is a temperature-sensitive self-replicating RNA comprising an Alphavirus replicon lacking a viral structural protein coding region.
84. The temperature-sensitive agent of embodiment 82 or embodiment 83, wherein the Alphavirus is selected from the group consisting of a Venezuelan equine encephalitis virus, a Sindbis virus, and a Semliki Forrest virus.
85. The temperature-sensitive agent of embodiment 82 or embodiment 83, wherein the Alphavirus is a Venezuelan equine encephalitis virus.
86. A method for transiently inducing a temperature-sensitive activity of a temperature-sensitive agent (ts-agent) in a subject, wherein the ts-agent is a temperature-sensitive viral vector or a temperature-sensitive self-replicating RNA comprising a heterologous nucleic acid comprising a coding region of human ZSCAN4, wherein one or more cells at or near the surface of the subject's body comprise the ts-agent, wherein the temperature-sensitive activity of the ts-agent comprises expression of human ZSCAN4 at a permissive temperature, and wherein the permissive temperature is the surface body temperature of the subject, comprising:

i) maintaining the surface body temperature of the subject at the permissive temperature for a period of time sufficient for the temperature-sensitive activity to induce an effect in the subject; and

ii) increasing the surface body temperature of the subject to a non-permissive temperature for a period of time sufficient for the temperature-sensitive activity to cease in the subject.

87. A method for transiently inducing a temperature-sensitive activity of a temperature-sensitive agent (ts-agent) in a subject, wherein the ts-agent is a temperature-sensitive viral vector or a temperature-sensitive self-replicating RNA comprising a heterologous nucleic acid comprising a coding region of human ZSCAN4, wherein the temperature-sensitive activity of the ts-agent comprises expression of human ZSCAN4 at a permissive temperature, and wherein the permissive temperature is the surface body temperature of the subject, comprising:

i) administering the ts-agent to one or more cells at or near the surface of the subject's body; and

ii) maintaining the surface body temperature of the subject at the permissive temperature for a period of time sufficient for the temperature-sensitive activity to induce an effect in the subject.

88. The method of embodiment 87, further comprising iii) increasing the surface body temperature of the subject to a non-permissive temperature for a period of time sufficient for the temperature-sensitive activity to cease in the subject.
89. The method of embodiment 86 or embodiment 87, wherein the temperature-sensitive agent is administered i) intradermally or subcutaneously; or ii) intramuscularly.
90. The method of embodiment 86 or embodiment 87, wherein the temperature-sensitive agent is administered intranasally.
91. The method of any one of embodiments 86-90, wherein the non-permissive temperature is above 36° C., and the permissive temperature is below 36° C., optionally wherein the permissive temperature is from about 31° C. to about 34° C., or about 33° C.±0.5° C., and the non-permissive temperature is 37° C.±0.5° C.
92. The method of any one of embodiments 86-91, wherein the effect of expression of human ZSCAN4 is prophylactic effect or a therapeutic effect.
93. The method of any one of embodiments 86-92, wherein the ts-agent is a temperature-sensitive viral vector and the temperature-sensitive activity further comprises replication and transcription of the temperature-sensitive viral vector.
94. The method of embodiment 93, wherein the temperature-sensitive viral vector is selected from the group consisting of a Sendai virus, an Adeno virus, an Adeno-associated virus, a Retrovirus, and an Alphavirus.
95. The method of embodiment 93, wherein the temperature-sensitive viral vector is an Alphavirus, optionally wherein the Alphavirus is selected from the group consisting of a Venezuelan equine encephalitis virus, a Sindbis virus, and a Semliki Forrest virus.
96. The method of embodiment 93, wherein the temperature-sensitive viral vector is a Sendai virus.
97. The method of any one of embodiments 86-92, wherein the ts-agent is a temperature-sensitive self-replicating RNA and the temperature-sensitive activity further comprises one or both of replication and transcription of the temperature-sensitive self-replicating RNA.
98. The method of embodiment 97, wherein the self-replicating RNA comprises an Alphavirus replicon lacking an Alphavirus viral structural protein coding region.
99. The method of embodiment 98, wherein the Alphavirus is selected from the group consisting of a Venezuelan equine encephalitis virus, a Sindbis virus, and a Semliki Forrest virus.
100. The method of embodiment 98, wherein the Alphavirus is a Venezuelan equine encephalitis virus.
101. The method of any one of embodiments 86-100, wherein the period of time sufficient for the temperature-sensitive activity to produce an effect ranges from about 12 hours to about 12 weeks, optionally wherein the period of time is from 1 to 7 days.
102. The method of any one of embodiments 86-100, wherein the period of time sufficient to induce an effect in the subject is from about 12 hours to about 7 days, optionally wherein the period of time is from about 12 hours to about 72 hours.
103. The method of any one of embodiments 86-102, wherein the subject is a mammalian subject, optionally wherein the subject is a human.

### EXAMPLES

Abbreviations: Aura (Aura virus); BFV (Barmah Forest virus); GFP (green fluorescent protein); GOI (gene of interest); IRES (internal ribosome entry site); LUC (luciferase); ONNV (O'nyong-nyong virus); RRV (Ross River virus); SeV (Sendai virus); SeVts (temperature-sensitive Sendai virus); SFV (Semliki Forest virus); shRNA (short hairpin RNA); SINV (Sindbis virus); srRNA (self-replicating RNA); ts (temperature-sensitive); ts-agent (temperature-sensitive-agent); VEEV (Venezuelan equine encephalitis virus); and WEEV (Western equine encephalitis virus).

The following examples are provided to illustrate certain particular features and/or embodiments. The examples are not intended to limit the disclosure as claimed.

### Example 1: Temperature-Sensitive Agents

This example describes a temperature-sensitive agent (ts-agent) that functions at a lower or higher temperature than normal body temperature, but does not function, or shows reduced functionality, at normal body temperature. The ts-agents are suitable for use in ex vivo, semi in vivo, and in vivo therapies. Temperature-sensitive viral vectors and self-replicating RNAs are engineered to express a gene of interest (GOI), a short hairpin RNA (shRNA), a long non-coding RNA, and/or other genetic elements. For instance, proteins with temperature-sensitive mutations, are functional at a lower temperature (e.g., at 30° C.), but are not functional at a normal body temperature (e.g., at 37° C.). Unless otherwise specified, normal body temperature is normal human body temperature of 37° C.±0.5° C.

A particular GOI is the ZSCAN4 gene, which is also referred to herein as a coding region of the ZSCAN4 gene, or a nucleic acid encoding the ZSCAN4 protein. The amino acid sequence of the human ZSCAN4 protein is set forth as (SEQ ID NO:38).

### Example 2: Temperature-Sensitive Sendai Virus Vectors (SeVts)

This example describes temperature-sensitive Sendai virus vectors (SeVts), which can be used for temperature-specific gene expression. Sendai virus vectors are based on the Sendai virus, a single-stranded RNA virus of the Paramyxovirus subfamily. SeV18/TS15ΔF is a temperature-sensitive Sendai virus vector, which allows for viral replication and gene expression when held at 32-35° C. However, viral replication ceases at non-permissive temperatures of 37° C. and above (Ban et al., PNAS 2011).

### Example 3: Temperature-Sensitive Self-Replicating RNAs (srRNAs)

This example describes the finding that a mutation in nsP2 protein encoded in a Venezuelan Equine Encephalitis Virus (VEEV) Vector exhibits temperature sensitivity. The temperature-sensitive system permits expression of a gene of interest (GOI) at 30° C.-33° C., but not at 37° C. and above. The srRNA vector permits higher expression of the GOI than a synthetic RNA encoding the GOI. The expression of the GOI is turned off, when the temperature shifted to 37° C. (e.g., a non-permissive temperature). A specific temperature-sensitive mutation (mutation 2) identified in this study is in the well-conserved region among Alphaviruses. Compared to Sendai Virus Vectors (SeVts), srRNAts may be more attractive for some applications, as srRNAts can be utilized in non-viral RNA expression systems.

### Materials and Methods

Cell Culture

A human Adipose Stem Cell-derived iPS cell line (ADSC-iPS cells) was purchased from System Biosciences (Palo Alto, Calif.). Cells were routinely maintained as undifferentiated human pluripotent cells (hPSCs) according to the standard hPSC culture method. Briefly, cells were cultured in StemFit basic02 (Ajinomoto, Japan) supplemented with 100 ng/ml FGF2. Further, cells were cultured on cell culture dishes coated with a laminin-511 substrate (iMatrix-511, Nippi, Japan).

VEEV Vector

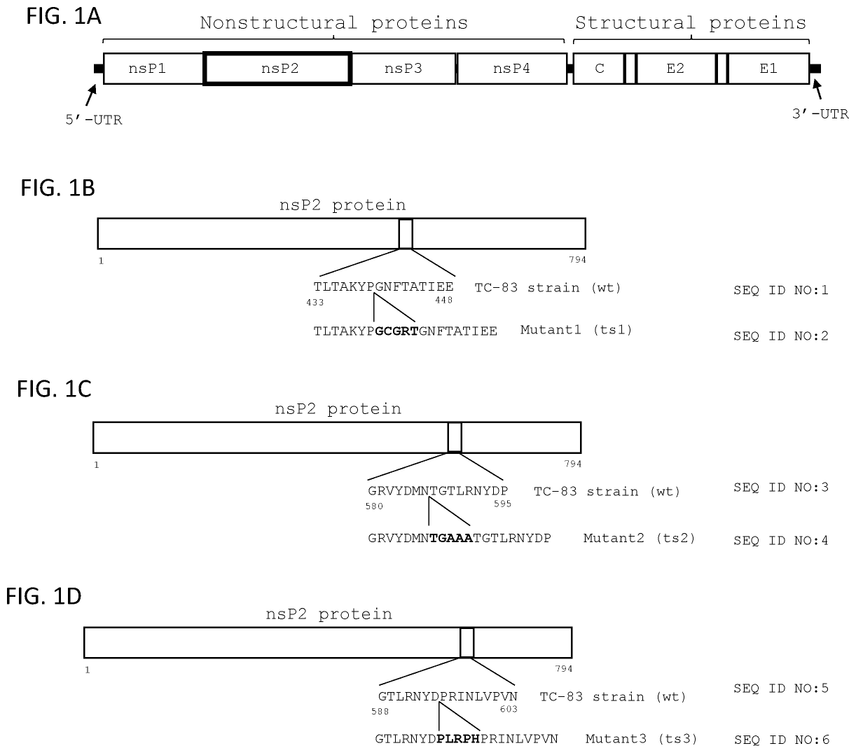

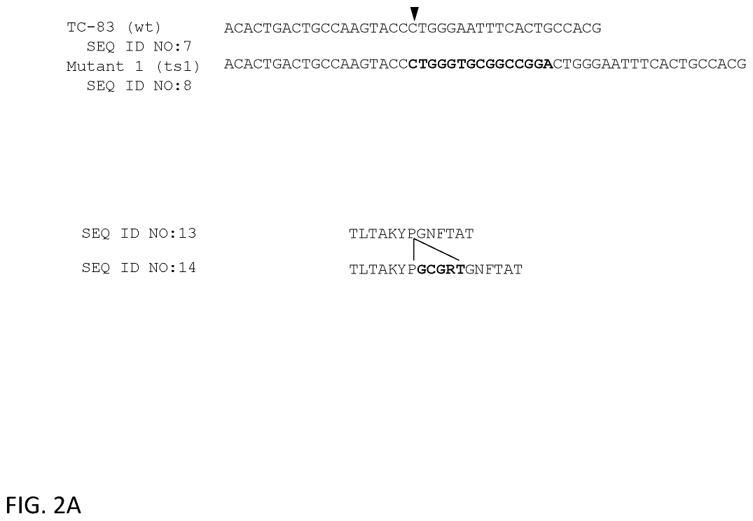

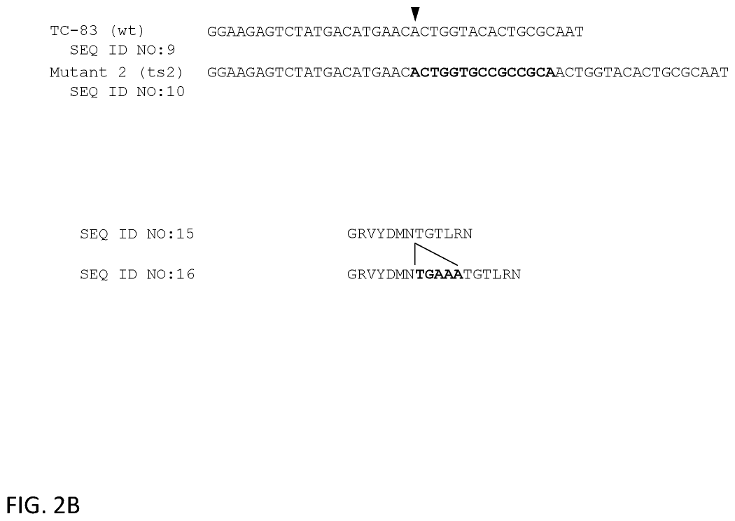

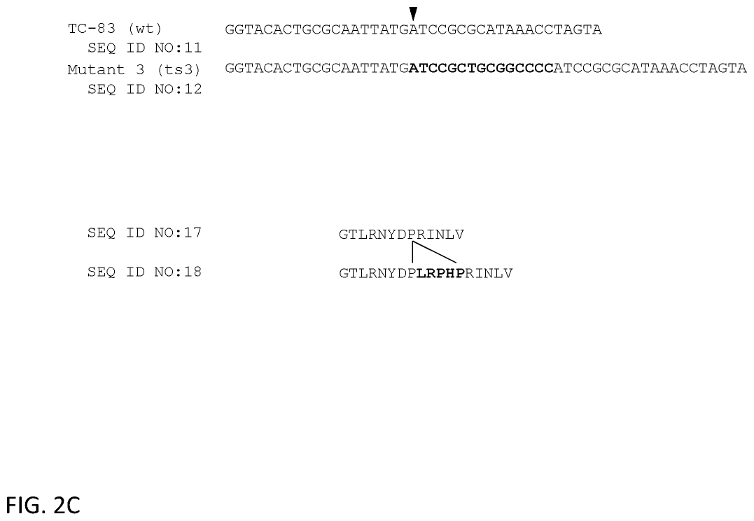

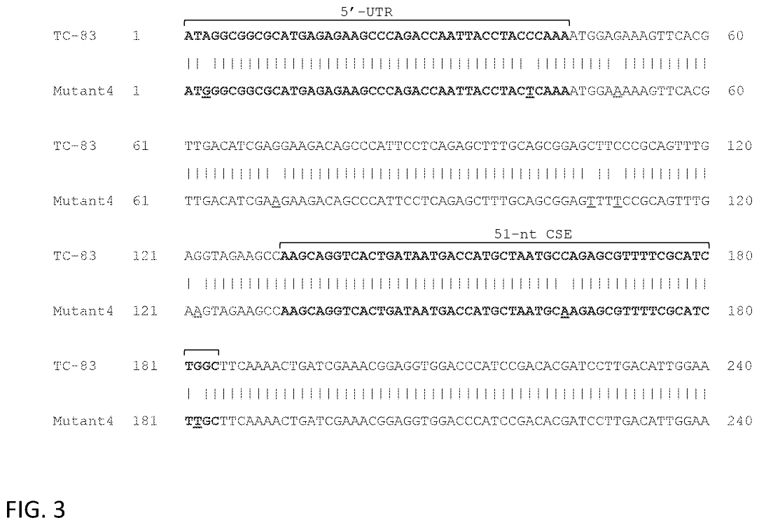

A VEEV vector plasmid was assembled using synthesized DNA fragments based on the publicly available sequence information (T7-VEE-IRES-Puro, herein after “srRNA1wt”). Per Yoshioka et al., 2013, the VEEV vector backbone was originally derived as in Petrakova et al., 2005. 7480 candidate sequences identified by insertional mutagenesis and massively parallel sequencing (Beitzel et al., 2010) were used to derive potential temperature-sensitive mutants. The original large-scale screen was performed by transposon-mediated insertion of 15 bp into the VEEV genome (FIG. 1A). Subsequently, a large number of 15-bp-insertion VEEV mutants that were able to proliferate at 30° C. or 40° C. were isolated. Although these data provided initial mutants for further research, it was not known whether these sequences exhibit temperature sensitivity, such as permissiveness at 32° C. or 33° C. and non-permissiveness at 37° C. Three mutant sequences—Mutant 1 (ts1, FIG. 1B), Mutant 2 (ts2, FIG. 1C), and Mutant 3 (ts3, FIG. 1D) were selected from a total of 7480 candidate mutant sequences (Data Set S1 from Beitzel et al., 2010). These mutant DNA fragments (FIG. 2) were synthesized and cloned into the VEEV vector and named srRNA1ts1 (mutant1), srRNA1ts2 (mutant 2), srRNA1ts3 (mutant 3). A mutant 4 was designed, which includes the 5′-region of virus sequence (5′-UTR and a part of N-terminal protein sequence of RNA-dependent RNA polymerase known to include a 51-nt conserved sequence element (CSE)). In this case, nucleotides were systematically changed to less thermo-stable variants (e.g., G->A), while maintaining the amino acid sequences (FIG. 3). The sequence of this region in srRNA1ts2 was replaced to generate srRNA1ts4 (i.e., containing both mutant 4 and mutant 2). Synthetic RNAs were produced from these vectors according to Yoshioka et al., 2013.

### Results

Assessing Temperature-Sensitivity of srRNAA1ts2-GFP and srRNA1ts3-GFP at 30° C., 32° C., and 37° C.

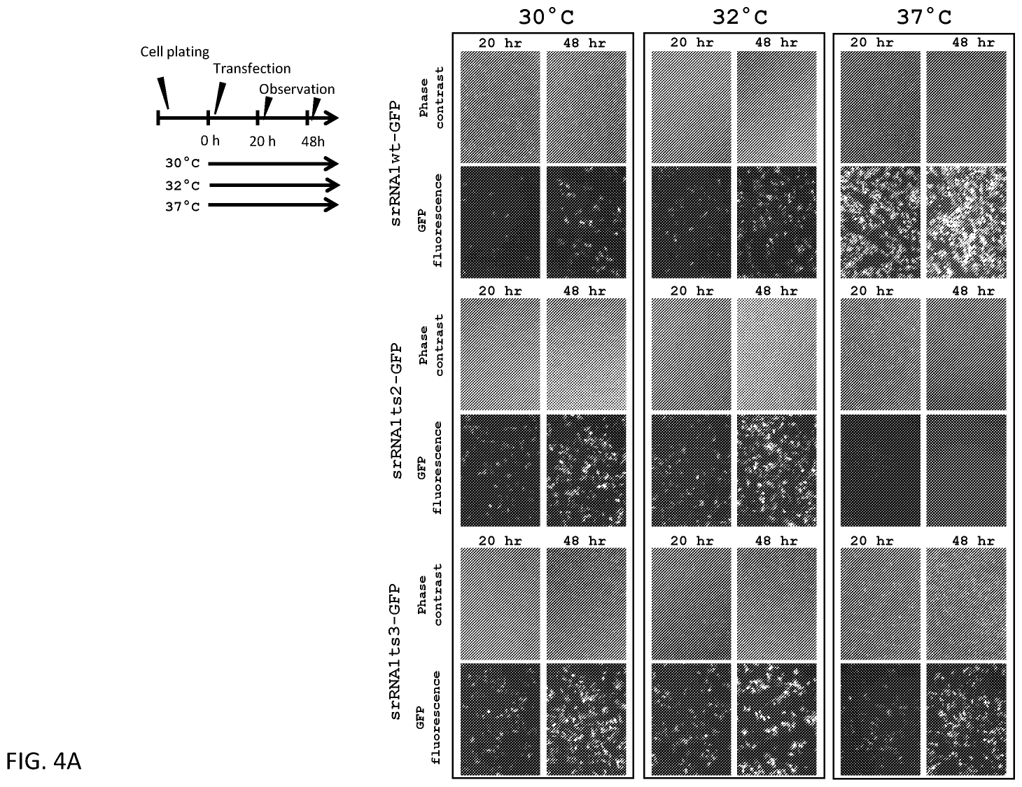

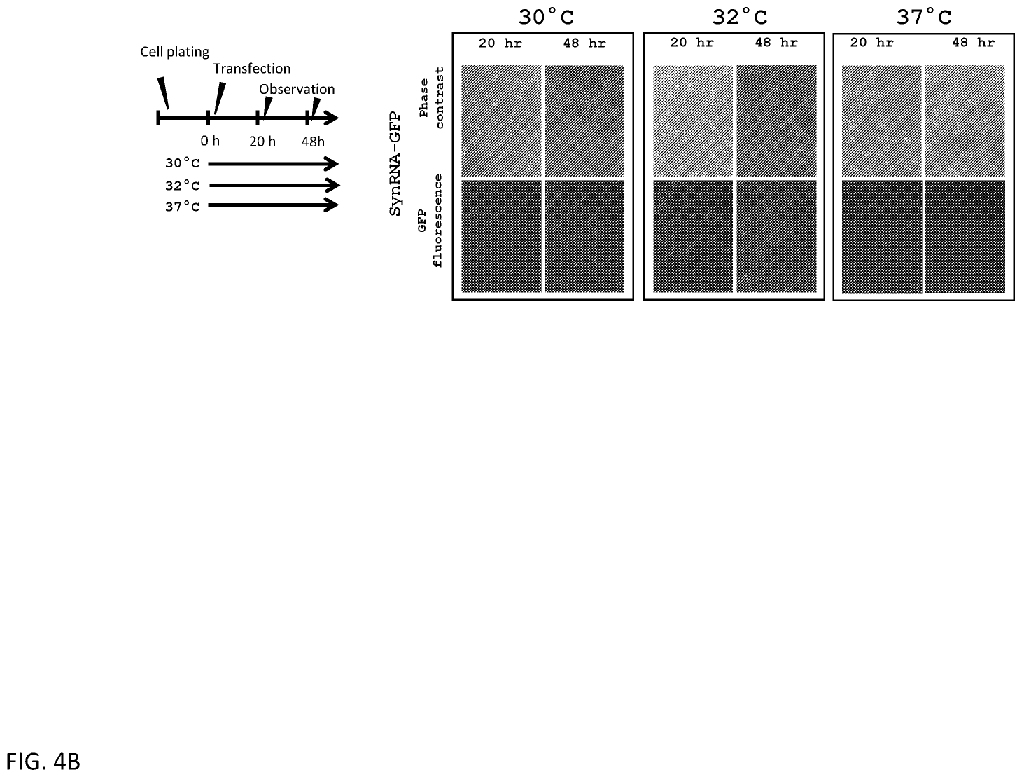

ADSC-iPSC cells were plated on a 24-well plate at a density of 80,000 cells/well. After 24 hours, cells were transfected with srRNA1wt-GFP, srRNA1ts2-GFP, or srRNA1ts3-GFP. For transfection, each well of a 24-well plate was treated with, 0.5 μg synthetic RNA (srRNA) mixed with 1 μl of JetMessenger (Polyplus) transfection reagent at a final volume of 50 μl. After adding the transfection complex to the cells, 450 μl of culture media was added. The cells were incubated at either 30° C., 32° C., or 37° C. At 6 hours after transfection, the medium was changed to remove the transfection complex. The phase-contrast and fluorescent images were taken at 20 hours and 48 hours. FIG. 4A shows that wild type (srRNA1wt-GFP) strongly expressed GFP at 37° C., but only weakly expressed GFP at both 30° C. and 32° C. By contrast, mutant 2 (srRNA1ts2-GFP) expressed GFP at 30° C. and 32° C., but not at 37° C. Mutant 3 (srRNA1ts3-GFP) expressed GFP at 30° C. and 32° C., but also expressed GFP at 37° C. Based on these results, mutant 2 was selected for further development. As expected, srRNA showed much higher expression of GFP, compared to the GFP expression levels that were achieved by a single transfection of synthetic mRNA encoding the GFP (FIG. 4B).

Assessing Temperature-Sensitivity of srRNA1 ts1-GFP and srRNA1ts2-GFP at 32° C.

ADSC-iPSC cells were plated on a 24-well plate at the density of 50,000 cells/well. After 24 hours, cells were transfected with srRNA1wt-GFP, srRNA1ts2-GFP, or srRNA1ts1-GFP. For transfection, each well of a 24-well plate was treated with, 0.5 μg synthetic RNA (srRNA) mixed with 1 μl of JetMessenger (Polyplus) transfection reagent at a final volume of 50 μl. After adding the transfection complex to the cells, 450 μl of culture media was added. The cells were incubated at 32° C. At 6 hours after transfection, the medium was changed to remove the transfection complex. The phase-contrast and fluorescent images were taken at 24, 48, 72, 96, 120, 144, 168, 192, 240, and 288 hours.

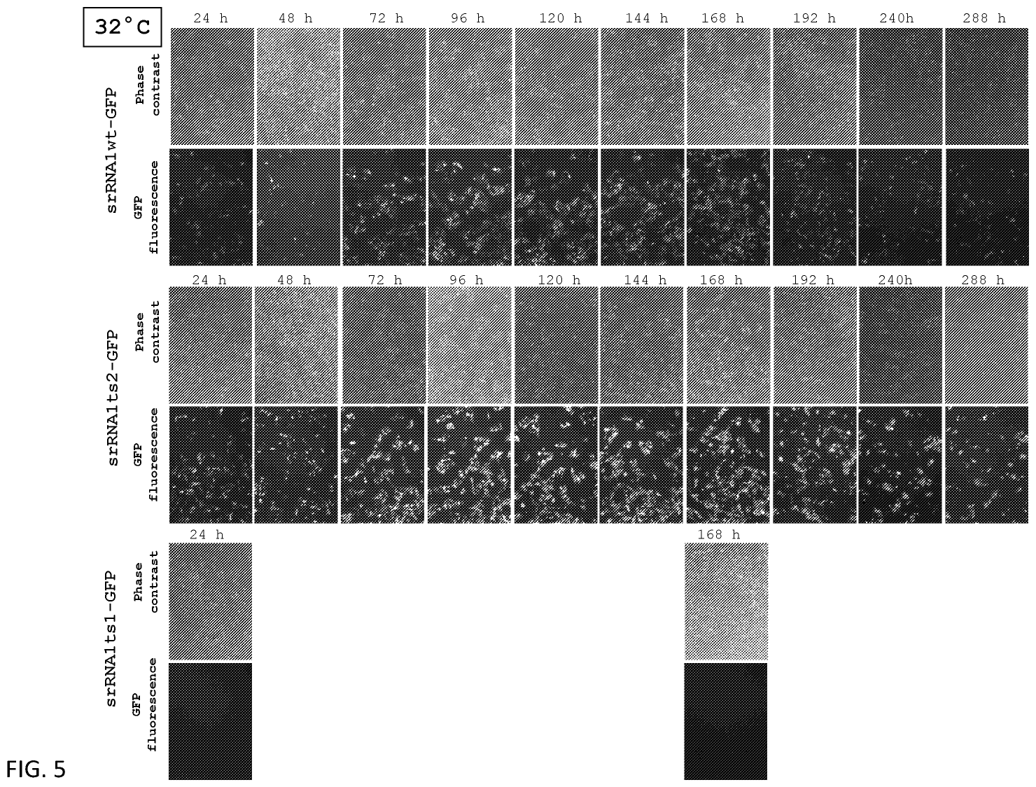

FIG. 5 shows the results. The GFP expression from wild type (srRNA1wt-GFP) started at 24 hours and continued until the end of the observation period (at 288 hours), but was very weak throughout the time course. By contrast, the GFP expression from a mutant 2 (srRNA1ts2-GFP) was very strong throughout the time course. Mutant 1 (srRNA1ts1-GFP) did not express GFP at all (based on observation at 24 hours and 168 hours). Based on these results, mutant 2 was selected for further development.

Assessing Temperature-Sensitivity of srRNA1 ts2-GFP and srRNA1ts4-GFP at 32° C., 33° C., and 37° C.

ADSC-iPSC cells were plated on a 24-well plate at the density of 50,000 cells/well. After 24 hours, cells were transfected with srRNA1ts2-GFP, or srRNA1ts4-GFP. For transfection, each well of a 24-well plate was treated with, 0.5 μg synthetic RNA (srRNA) mixed with 1 μl of JetMessenger (Polyplus) transfection reagent at a final volume of 50 μl. After adding the transfection complex to the cells, 450 μl of culture media was added. The cells were incubated at either 32° C., 33° C., or 37° C. At 6 hours after transfection, the medium was changed to remove the transfection complex. The phase-contrast and fluorescent images were taken at 20, 48, and 96 hours.

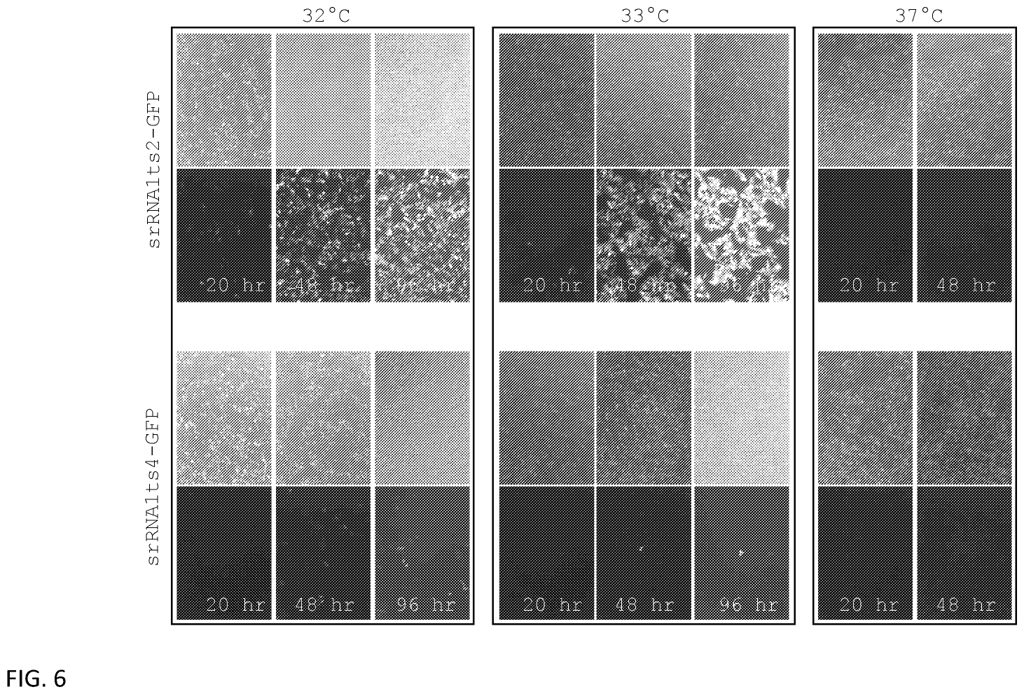

FIG. 6 shows the results. At 32° C. and 33° C., the GFP expression from mutant 2 (srRNA1ts2-GFP) started as early as 20 hours, but significantly increased at 48 hours, and further increased at 96 hours. The expression of GFP was stronger at 33° C. than at 32° C. Consistent with the experiments above, GFP was not expressed at all at 37° C. The srRNA1ts4-GFP (containing both a mutant 2 and mutant 4) showed a similar temperature profile to srRNA1ts2-GFP, but the GFP expression was much weaker overall. Based on these results, mutant 2 was selected for further development.

Assessing Temperature-Sensitivity of srRNA1ts2-GFP at 32° C.

ADSC-iPSC cells were plated on a 24-well plate at the density of 80,000 cells/well. After 24 hours, cells were transfected with srRNA1ts2-GFP. For transfection, each well of a 24-well plate was treated with, 0.5 μg synthetic RNA (srRNA) mixed with 1 μl of JetMessenger (Polyplus) transfection reagent at a final volume of 50 μl. After adding the transfection complex to the cells, 450 μl of culture media was added. The cells were incubated at 32° C. At 6 hours after transfection, the medium was changed to remove the transfection complex. The medium was changed every day. The srRNA1ts2-GFP vector contains a puromycin N-acetyltransferase (pac) selection gene inserted after the “IRES” sequence, and thus, can be selected using puromycin. The experiments were done in the absence (upper panel) or presence (lower panel) of 1 μg/ml of puromycin. For the cells with puromycin selection, puromycin was added at 48 hours and 72 hours. The phase-contrast and fluorescent images were taken at 24, 48, 72, 96, 144, 168, 192 hours.

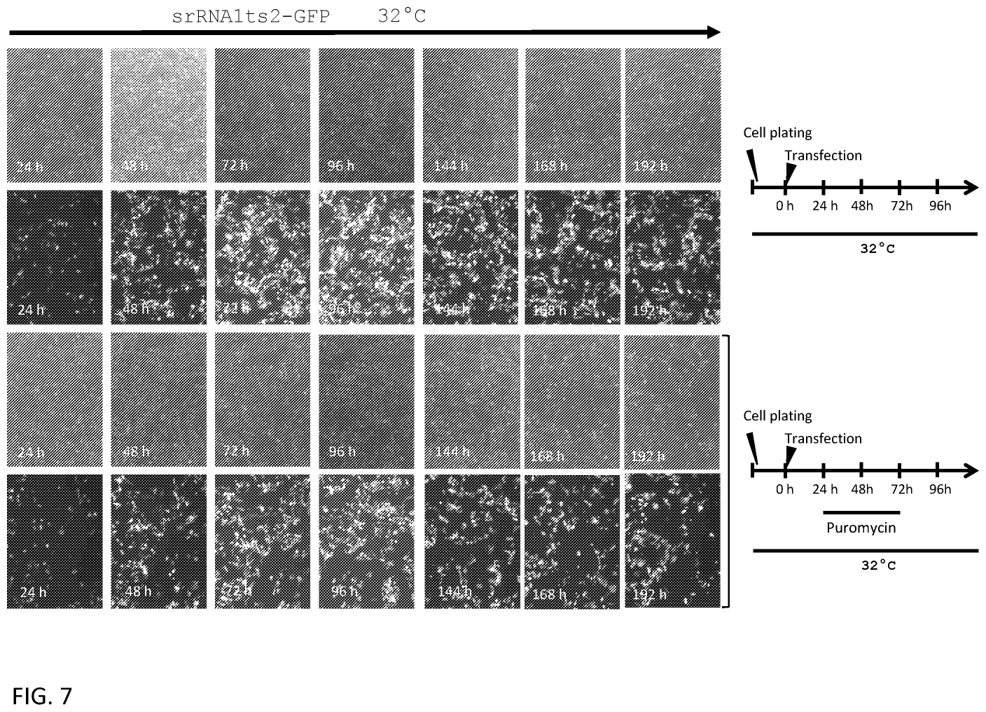

FIG. 7 shows the results. At 32° C., the GFP expression from srRNA1ts2-GFP started as early as 24 hours, but significantly increased at 48 hours, and peaked at 72 hours and 96 hours. The expression of GFP continued until the end of observation period (at 192 hours). The expression pattern of GFP did not seem to be altered by the addition of puromycin.

Assessing Temperature-Sensitivity of srRNA1ts2-GFP Switched From 32° C. to 37° C. After 24 hours.

ADSC-iPSC cells were plated on a 24-well plate at the density of 80,000 cells/well. After 24 hours, cells were transfected with srRNA1ts2-GFP. For transfection, each well of a 24-well plate was treated with, 0.5 μg synthetic RNA (srRNA) mixed with 1 μl of JetMessenger (Polyplus) transfection reagent at a final volume of 50 μl. After adding the transfection complex to the cells, 450 μl of culture media was added. The cells were incubated at 32° C. At 6 hours after transfection, the medium was changed to remove the transfection complex. The medium was changed every day. The srRNA1ts2-GFP vector contains a puromycin N-acetyltransferase (pac) selection gene inserted after the “IRES” sequence, and thus, can be selected using puromycin. The experiments were done in the absence (upper panel) or presence (lower panel) of 1 μg/ml of puromycin. For the cells with puromycin selection, puromycin was added at 48 hours and 72 hours. To test the effects of temperature shift, the cell cultures were transferred to a C02 incubator maintained at 37° C. at 24 hours (24 hours after the transfection). The phase-contrast and fluorescent images were taken at 24, 48, 72, 96, 144, 168, 192 hours.

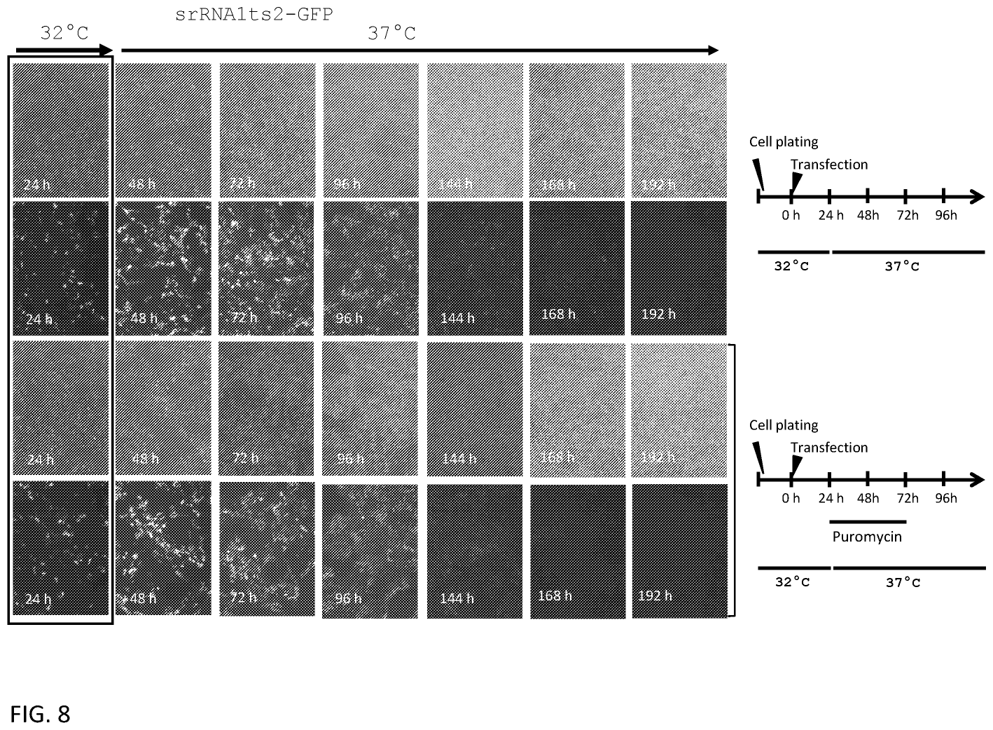

FIG. 8 shows the results. At 32° C., the GFP expression from srRNA1ts2-GFP started as early as 24 hours, but continued to increase even after the switching of temperature to 37° C. at 24 hours. The expression of GFP peaked at 48, and then, started to decrease. By 96 hours, the GFP expression became very weak and by 144 hours the GFP expression could not be detected any more. Subsequently, there was no GFP expression until the end of observation period at 192 hours. Thus, the expression of the GOI (represented here by GFP) was rapidly turned off, when the temperature shifted from 33° C. (a permissive temperature) to 37° C. (a non-permissive temperature). The expression pattern of GFP did not seem to be altered by the addition of puromycin.

Assessing Temperature-Sensitivity of srRNA1ts2-GFP Switched from 32° C. to 37° C. After 48 hours.

ADSC-iPSC cells were plated on a 24-well plate at the density of 80,000 cells/well. After 24 hours, cells were transfected with srRNA1ts2-GFP. For transfection, each well of a 24-well plate was treated with, 0.5 μg synthetic RNA (srRNA) mixed with 1 μl of JetMessenger (Polyplus) transfection reagent at a final volume of 50 μl. After adding the transfection complex to the cells, 450 μl of culture media was added. The cells were incubated at 32° C. At 6 hours after transfection, the medium was changed to remove the transfection complex. The medium was changed every day. The srRNA1ts2-GFP vector contains a puromycin N-acetyltransferase (pac) selection gene inserted after the “IRES” sequence, and thus, can be selected using puromycin. The experiments were done in the absence (upper panel) or presence (lower panel) of 1 μg/ml of puromycin. For the cells with puromycin selection, puromycin was added at 48 hours and 72 hours. To test the effects of temperature shift, the cell cultures were transferred to a C02 incubator maintained at 37° C. at 48 hours (48 hours after the transfection). The phase-contrast and fluorescent images were taken at 24, 48, 72, 96, 144, 168, 192 hours.

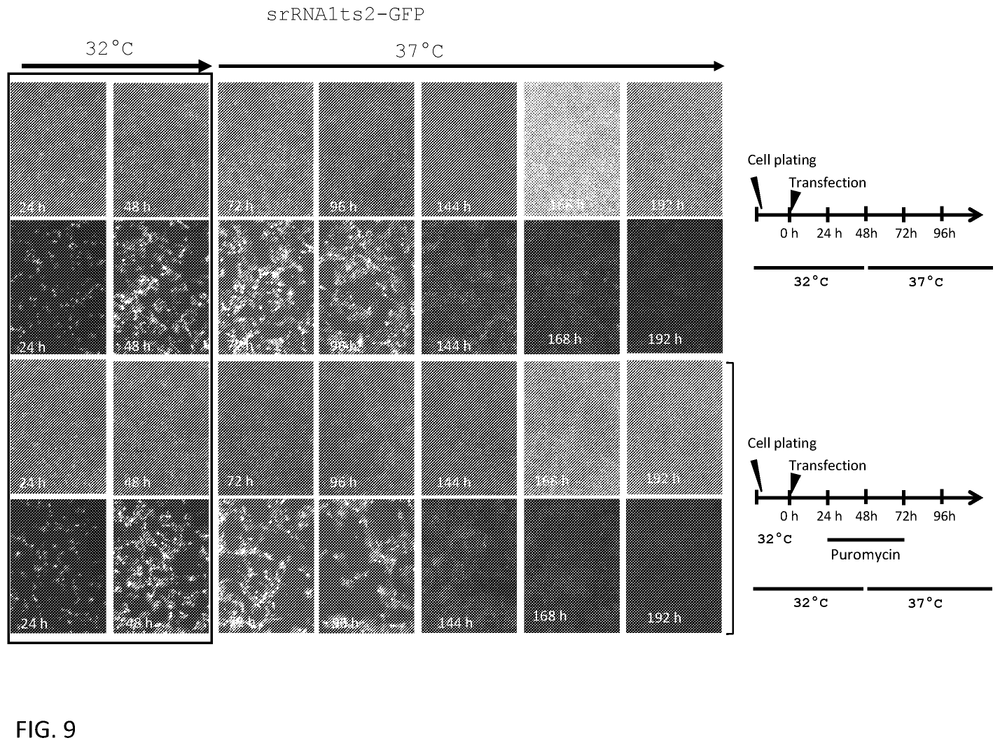

FIG. 9 shows the results. At 32° C., the GFP expression from srRNA1ts2-GFP started as early as 24 hours and further increased at 48 hours. The expression of GFP continued until 96 hours even after the switching of temperature to 37° C. at 48 hours. But the GFP expression started to decrease from 72 hours and by 96 hours the GFP expression became very weak. By 144 hours the GFP expression was barely detected and completely turned off by 192 hours. Thus, the expression of the GOI (represented here by GFP) was rapidly turned off, when the temperature shifted from 33° C. (a permissive temperature) to 37° C. (a non-permissive temperature). The expression pattern of GFP did not seem to be altered by the addition of puromycin.

Assessing Temperature-Sensitivity of srRNA1ts2-GFP Switched from 32° C. to 37° C. after 72 Hours.

ADSC-iPSC cells were plated on a 24-well plate at the density of 80,000 cells/well. After 24 hours, cells were transfected with srRNA1ts2-GFP. For transfection, each well of a 24-well plate was treated with, 0.5 μg synthetic RNA (srRNA) mixed with 1 μl of JetMessenger (Polyplus) transfection reagent at a final volume of 50 μl. After adding the transfection complex to the cells, 450 μl of culture media was added. The cells were incubated at 32° C. At 6 hours after transfection, the medium was changed to remove the transfection complex. The medium was changed every day. The srRNA1ts2-GFP vector contains a puromycin N-acetyltransferase (pac) selection gene inserted after the “IRES” sequence, and thus, can be selected using puromycin. The experiments were done in the absence (upper panel) or presence (lower panel) of 1 μg/ml of puromycin. For the cells with puromycin selection, puromycin was added at 48 hours and 72 hours. To test the effects of temperature shift, the cell cultures were transferred to a C02 incubator maintained at 37° C. at 72 hours (72 hours after the transfection). The phase-contrast and fluorescent images were taken at 24, 48, 72, 96, 144, 168, 192 hours.

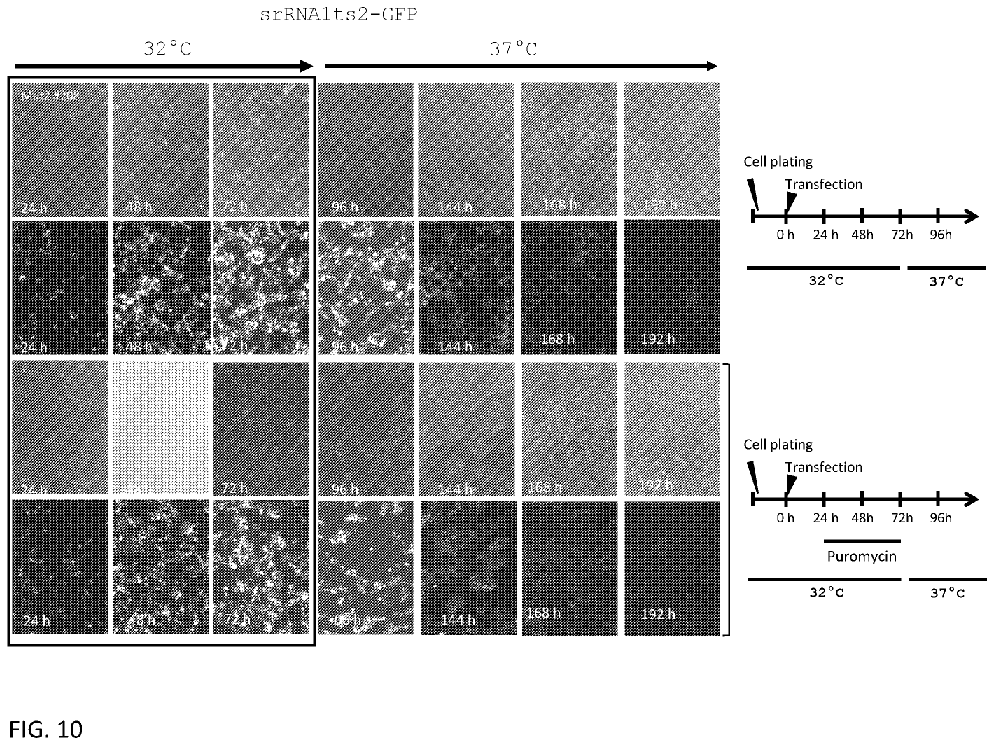

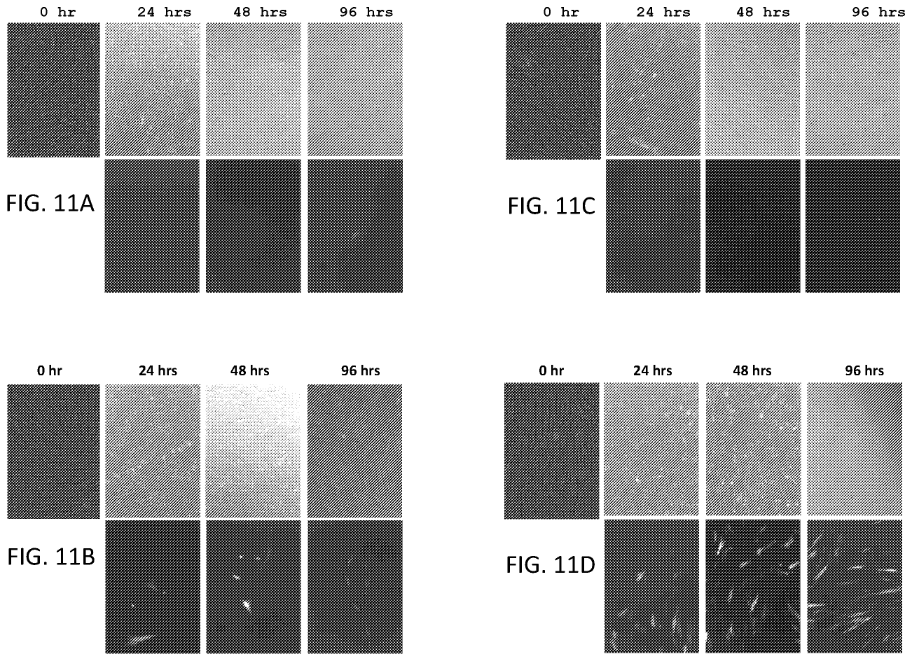

FIG. 10 shows the results. At 32° C., the GFP expression from srRNA1ts2-GFP started as early as 24 hours and further increased at 48 hours. The expression of GFP continued until 96 hours even after the switching of temperature to 37° C. at 48 hours. But the GFP expression started to decrease from 72 hours and by 144 hours the GFP expression became very weak. By 168 hours the GFP expression was barely detected and completely turned off by 192 hours. Thus, the expression of the GOI (represented here by GFP) was rapidly turned off, when the temperature shifted from 33° C. (permissive temperature) to 37° C. (a non-permissive temperature). The expression pattern of GFP did not seem to be altered by the addition of puromycin.

Assessing Temperature-Sensitivity of srRNA1ts2-GFP in Fibroblast Cells

Human newborn dermal fibroblast cells (HDFn at passage 20) were plated on a 24-well plate at the density of 10,000 cells/well. After 24 hours, cells were transfected with srRNA1wt-GFP. Transfection of srRNA1wt-GFP (0.5 μg synthetic RNA) was carried out by using either JetMessenger (Polyplus) transfection reagent or Lipofectamine MessengerMax (Thermo-Fisher). The cells were incubated at 37° C. To see the effect of B18R, which is known to repress interferon responses, the transfection and cell culture were carried out in the absence (upper panel) or presence (lower panel) of 200 ng/ml B18R. The medium was changed every day. The phase-contrast and fluorescent images were taken at 0, 24, 48, and 96 hours.

FIG. 11 shows the results. In the absence of B18R, almost no expression of GFP was detected. By contrast, in the presence of B18R, the GFP expression from srRNA1wt-GFP started as early as 24 hours and continued until 48 hours and 72 hours. The expression of GFP was strong in the GFP+ cells, but the frequency of GFP+ cells was not high. This was most likely due to the low transfection efficiency of srRNA1wt-GFP on human primary fibroblast cells.

Aligning Amino Acid Sequences of Alphavirus Family Corresponding to Mutant 2 (ts2)

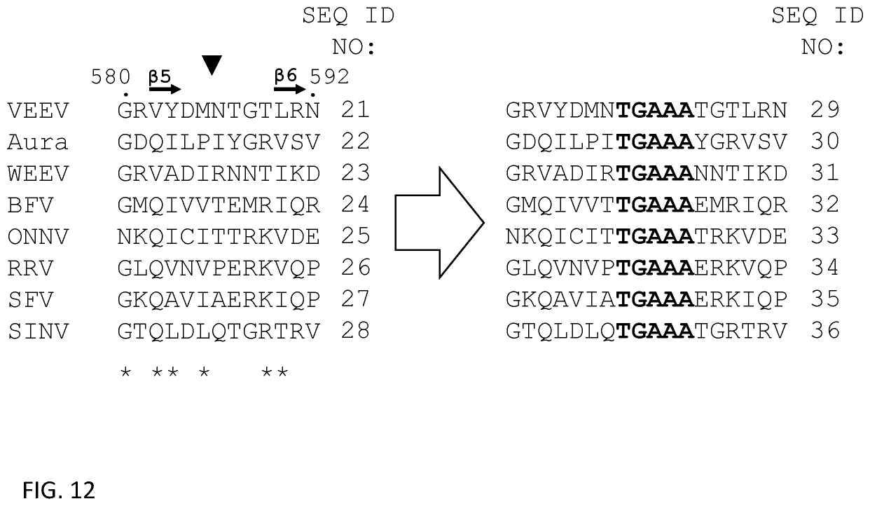

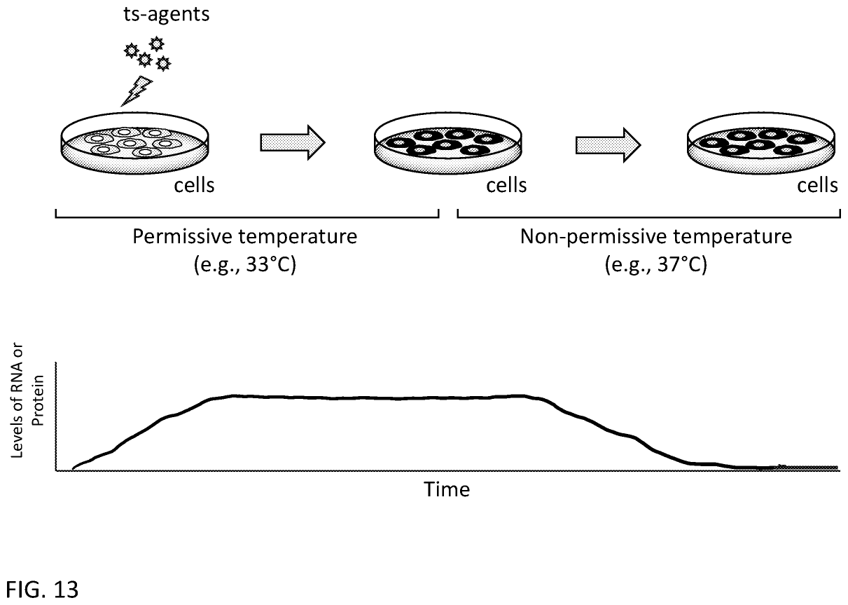

As shown in FIG. 12, the structure of nsP2 proteins of Alphavirus, even at the amino acid level, is well conserved among family members. Based on the 3D structural model (Russo et al., 2006), the protein region, where the 5 amino acids SEQ ID NO:44 (TGAAA) are inserted in the mutant 2, is a turning point between two beta-sheet structures, which is also well conserved among Alphavirus family members. Therefore, it is highly likely that the temperature-sensitivity of mutant 2 is transferable to other Alphavirus family members, including Aura (Aura virus), WEEV (Western equine encephalitis virus), BFV (Barmah Forest virus), ONNV (O'nyong-nyong virus), RRV (Ross River virus), SFV (Semliki Forest virus), and SINV (Sindbis virus). Suitable locations for insertions in nsP2 of various Alphaviruses for conferring temperature-sensitivity are listed in Table 3-1.

TABLE 3-1
Alphavirus nsP2 Sequences and Insertion Sites

| | NCBI | Insertion Site (:) |
|---|---|---|
| Alphavirus | Accession No. | amino acids positions |
| Venezuelan equine | NP_740697 | 586:587 |
| encephalitis virus | | |
| Aura virus | NP_819011 | 596:597 |
| Western equine | CAA52868 | 683:684 |
| encephalitis virus | | |
| Barmah Forest virus | NP_818996 | 588:589 |
| Onyong-nyong virus | AAC97204 | 1123:1124 |
| Ross River virus | NP_740679 | 587:588 |
| Semliki Forest virus | NP_463457 | 1125:1126 |
| Sindbis virus | AF492770_1 | 1137:1138 |

### Example 4: Temperature-Sensitive Antibodies

This example describes temperature-sensitive antibodies. An antibody that functions at a permissive temperature (e.g., 32° C.) and does not function or shows reduced function at a non-permissive temperature (e.g., 37° C.) is engineered by insertion or substitution of amino acid sequences. A temperature-sensitive antibody can be produced by inserting a linker oligonucleotide encoding the temperature-sensitive helix-coil transition peptide (-Glu-Ala-Ala-Ala-Lys-, set forth as SEQ ID NO:37), as described (Kamihara and Iijima, 2000; Merutka and Stellwagen, 1990). In this way, an engineered antibody can be produced, which functions at a permissive temperature (e.g., 32° C.), but does not function at a non-permissive temperature (e.g., 37° C.). Alternatively, the antibody DNA sequence of animals naturally living in a low temperature environment (e.g., Atlantic salmon and shrimp) can be used, as these antibodies are optimally functioning at a permissive temperature (at low temperature), but show reduced functionality at a non-permissive temperature (e.g., 37° C.).

### Example 5: Temperature-Sensitive Proteins

This example describes temperature-sensitive proteins. Such proteins function at a permissive temperature (e.g., 32° C.) and do not function or show low function at a non-permissive temperature (e.g., 37° C.). Temperature-sensitive proteins are engineered by substituting amino acid sequences. Alternatively, temperature-sensitive proteins obtained from animals naturally living in a low temperature environment (e.g., Atlantic salmon and shrimp) can be used, as these proteins are optimally functioning at a permissive temperature (at low temperature), but show reduced functionality at a non-permissive temperature (e.g., 37° C.).

### Example 6: Temperature-Sensitive RNAs

This example describes temperature-sensitive RNA molecules. RNA molecules include, but are not limited to, mRNA, a precursor of mRNA, non-coding RNA, siRNA, and shRNA. Temperature-sensitive RNAs function at a permissive temperature (e.g., 32° C.) and do not function or show low function at a non-permissive temperature (e.g., 37° C.). Temperature-sensitive RNAs are engineered by systematically changing the nucleotides of RNA molecules to less thermo-stable variants (e.g., G->A), while ensuring that the functional properties of the RNAs are maintained. Further, the difference in thermostability of the nucleotide pairs, induced by a shift in temperature, changes the secondary structure of the RNAs.

### Example 7: Ex Vivo Treatment of Cells with Temperature-Sensitive Agents

This example demonstrates a method for transiently delivering an RNA or protein to cells ex vivo (FIG. 13). A temperature-sensitive therapeutic agent can be any of the temperature-sensitive therapeutic agents disclosed herein. Ts-agents such as srRNAs or Sendai virus vector are functional at a permissive temperature (e.g., 33° C.), but non-functional at a non-permissive temperature (e.g., 37° C.). Target cells treated with the ts-agent are cultured ex vivo at a permissive temperature for a certain duration (e.g., 3 days), and then are cultured at a non-permissive temperature for a certain duration (e.g., 10 days). Levels of RNAs (or proteins translated from the RNAs) of a GOI increase at a permissive temperature and reach a high level. After switching to a non-permissive temperature, expected levels of RNAs gradually decrease and subsequently reach to a non-expression level (FIG. 13).

### Example 8: Ex Vivo Therapeutic Use of Temperature-Sensitive Agents

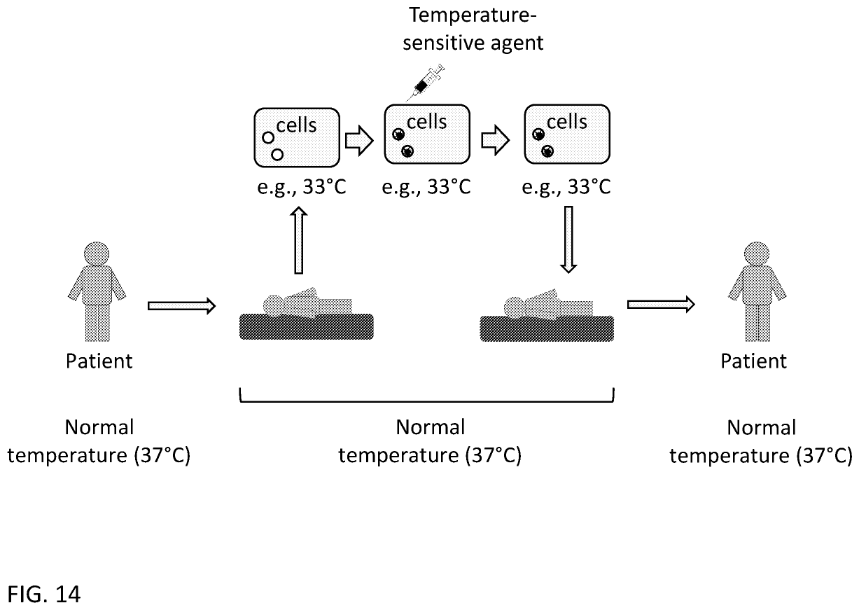

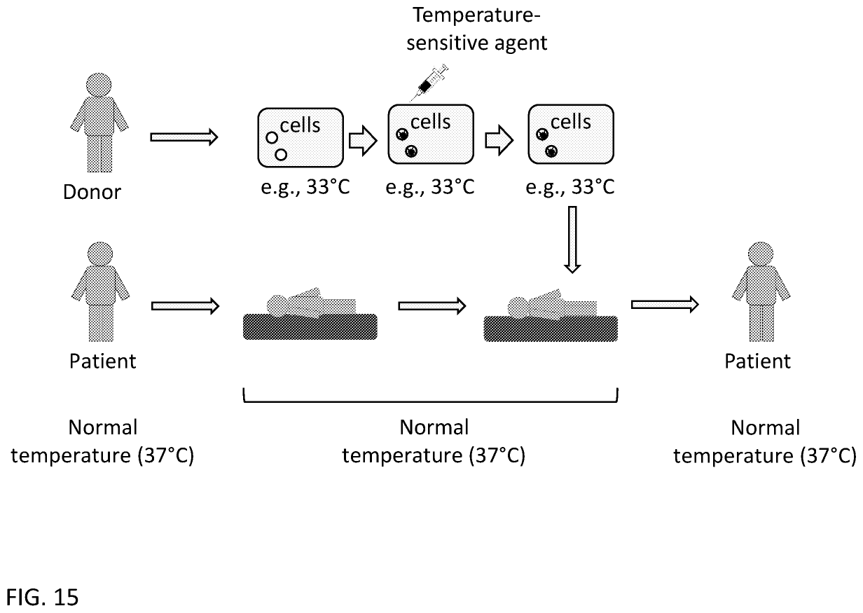

This example demonstrates a method for transiently delivering an RNA or protein to cells ex vivo (FIG. 14 and FIG. 15). Ts-agents such as srRNAs or Sendai virus vector are functional at a permissive temperature (e.g., 33° C.), but non-functional at a non-permissive temperature (e.g., 37° C.: a human body temperature). Typically, target cells are taken from a patient (autologous cell transplants; FIG. 14), but it is also possible to use target cells isolated from a donor (allogenic cell transplant; FIG. 15). For instance, the target cells may be isolated by using antibody-conjugated magnetic beads. Target cells are incubated with the ts-agent ex vivo at a permissive temperature, e.g., at 33° C. for a certain duration, e.g., 24 hours. Levels of RNAs (or proteins translated from the RNAs) of a GOI increase at a permissive temperature reach a high level. After the therapeutic effect is induced, the cells are transplanted back to the patient in order to treat the patient. The activity of the temperature-sensitive therapeutic agent is not induced at the subject's normal body temperature (i.e. normal body temperature is a non-permissive temperature). The degradation of the temperature-sensitive therapeutic agent begins after the therapeutic effect is induced, and eventually the temperature-sensitive therapeutic agent is completely degraded. The body temperature is maintained at or above 37° C. for the lifetime of the patient, and thus, the ts-agent is not reactivated and cells other than the target cells will not be treated with the ts-agent.

Mobilized Human Peripheral Blood Cells

Human blood cells isolated from a patient's, or donor's, bone marrow or peripheral blood are treated with ts-agents ex vivo at a permissive temperature. After injection of G-CSF or other mobilizing agents, human white blood cells are collected from peripheral blood by an apheresis machine (e.g., COBE Spectra). The white blood cells collected after mobilization from bone marrow contain granulocytes, monocytes, lymphocytes, dendritic cells, mesenchymal stem cells (MSCs), vascular endothelial cells (VECs), and CD34+ hematopoietic/progenitor cells. The treatment of these cells with ts-agents is conducted ex vivo, ideally, using a functionally closed system such as Miltenyi's CliniMacs Prodigy, at a functional temperature (e.g., 33° C.) for a certain duration (a few hours to a few weeks). Subsequently, the treated cells are infused into patients at a non-permissive temperature (37° C.). The ts-agents, cells containing the ts-agents, or the product of ts-agents do not function in the patient's body.

Human CD34+ Hematopoietic Stem Progenitor Cells

Human CD34+ hematopoietic stem/progenitor cells are isolated from the mobilized human peripheral blood cells or bone marrow cells by antibody (against CD34)-conjugated magnetic beads and used as target cells are treated with ts-agents ex vivo at a permissive temperature. After treatment with a ts-agent, human CD34+ cells are infused into a patient's body and engraft in the patient's bone marrow. These cells will eventually produce all the blood cells in the patient's body, and thus, are a suitable target for a variety of diseases.

Any Human Cells Including Tissue Stem Cells

Any human cells isolated from patient or donor and used as target cells are treated with ts-agents ex vivo at a permissive temperature. Such cells include but are not limited to skin fibroblast cells, follicular cells, skeletal muscle cells, hepatocytes, and neural tissues. Such cells also include a variety of tissue stem cells such as mesenchymal stem cells, neural stem cells, muscle stem cells, skin stem cells, and intestinal stem cells.

### Example 9: Semi In Vivo Therapeutic Use of Temperature-Sensitive Agents

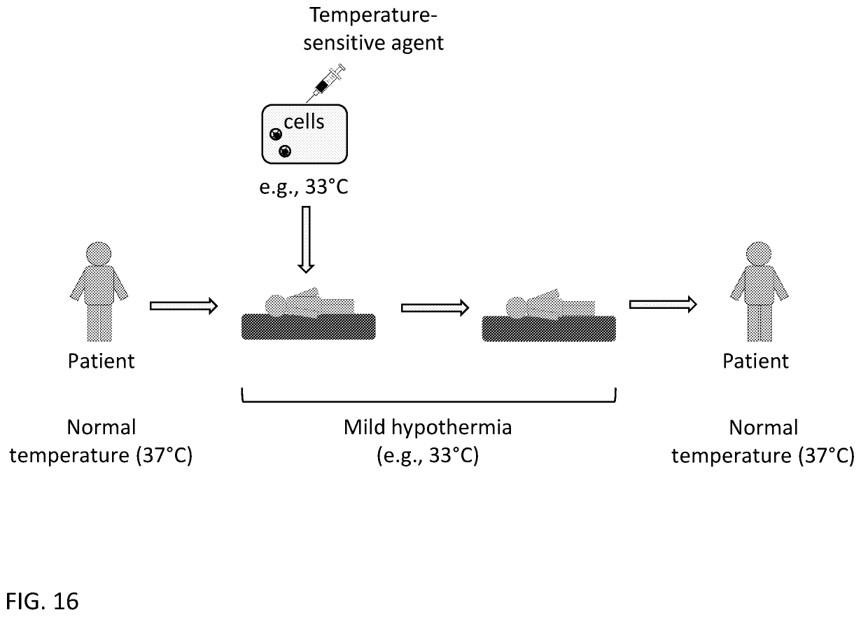

This example describes a semi in vivo method for transiently delivering an RNA or protein to cells (FIG. 16). A temperature-sensitive therapeutic agent can be any of the temperature-sensitive therapeutic agents disclosed herein. A ts-agent is functional at a permissive temperature (e.g., 33° C.), but non-functional at a non-permissive temperature (e.g., 37° C.).

A patient undergoes a procedure for therapeutic hypothermia: the patient's core body temperature is maintained at a temperature lower than normal body temperature (e.g., 33° C.). Target cells (any cells—autologous or allogenic) are treated by the ts-agent ex vivo and immediately infused into the patient's circulation or injected into the patient's organs.

While the patient is maintained at the targeted temperature, e.g., 33° C. for some time, e.g., 24 hours, the ts-agent exhibits their expected functions. Levels of RNAs (or proteins translated from the RNAs) of a GOI increase at a permissive temperature reach a high level. Subsequently, the patient's body temperature is returned to normal temperature at 37° C. The ts-agent no longer functions at the non-permissive condition, 37° C. inside the patient's body. The body temperature is maintained at or above 37° C. for the lifetime of the patient, and thus, the ts-agent is not reactivated and cells other than the target cells will not be treated with the ts-agent. Notably, this therapeutic procedure can be applied to any cell type including those described above.

### Example 10: In Vivo Therapeutic Use of Temperature-Sensitive Agents

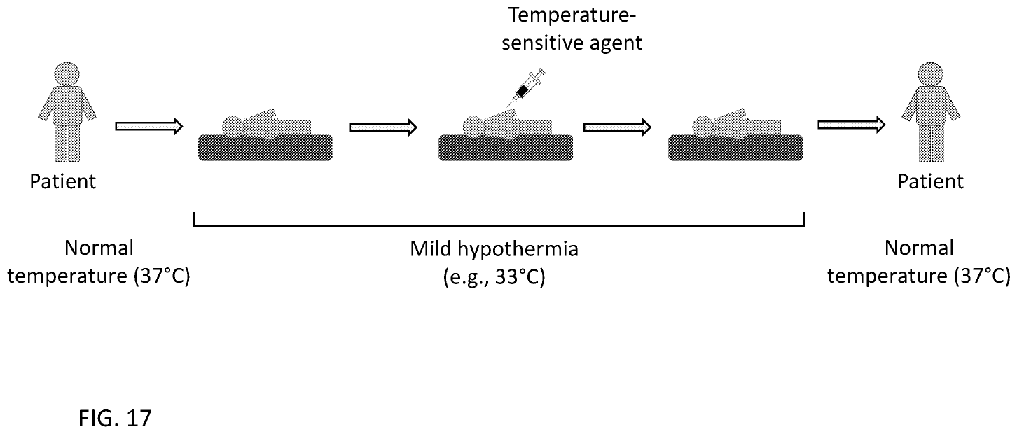

This example demonstrates how a temperature-sensitive viral vector is administered to a subject and transiently activated when mild hypothermia is induced in the subject (FIG. 17). A temperature-sensitive therapeutic agent can be any of the temperature-sensitive therapeutic agents disclosed herein. Temperature-sensitive therapeutic agents are functional at a permissive temperature (e.g., 33° C.), but non-functional at a non-permissive temperature (e.g., 37° C.: a human body temperature).

The subject's body temperature is lowered using a target-temperature management (TTM) procedure, which has been used in the clinic for patients with heart and brain trauma (Callaway et al., 2015). A TTM procedure is designed to achieve and maintain a specific body temperature in a subject for a duration of time. Such procedures have previously been used therapeutically to reduce the negative effects resulting from various acute health issues such as heart attacks and strokes. Equipment and general methods of using a TTM procedure are known in the art and can be used with the methods described herein. The TTM procedure can be carried out using a number of methods, including cooling catheters, cooling blankets, and application of ice around the body. A variety of instruments have been used for such purposes. For example, the ArcticSun™ is an instrument that can be used to decrease or increase a patient's body temperature to between 32° C.-38.5° C. (Pittl et al., 2013). The procedure can be performed safely and it has been reported that there are no major adverse effects that are caused by this instrument.

A patient is placed under hypothermic conditions using the TTM procedure, and the target body temperature is one sufficient to induce an activity of the temperature-sensitive therapeutic agent. The temperature-sensitive therapeutic agent is delivered directly to the patient through either the systemic route (e.g., intravenously) or through direct injection into organs/tissues (e.g., catheter, or percutaneous needle injection) (FIG. 17).

The patient's temperature is kept at the permissive temperature for a time sufficient to allow induction of a desired activity of the temperature-sensitive therapeutic agent. The desired activity of the temperature-sensitive agent leads to a therapeutic effect in the cells containing or exposed to the temperature-sensitive therapeutic agent.

After the desired therapeutic effect is achieved, the patient's body temperature is then returned to a normal body temperature (i.e., a non-permissive temperature) causing the activity of the temperature-sensitive therapeutic agent to cease. This is followed by degradation of temperature-sensitive therapeutic agent.

Systemic Delivery Through Circulation

A patient is placed under hypothermic conditions (e.g., at 33° C.). Once the patient's core body temperature is maintained at the target temperature stably, a ts-agent is delivered directly to the patient intravenously. The ts-agent is delivered to many organs and tissues through this systemic route. The core body temperature of the patient is maintained at the functional temperature for a desired duration (e.g., 24 hours). While the patient's body temperature is kept at a permissive temperature for the agent (e.g., at 33° C.), the agent is functioning. When the patient's body temperature is returned to normal at 37° C., which is a non-permissive temperature of the agent, the agent stops working.

The ts-agent may be a naked RNA (i.e., a synthetic RNA). Systemic delivery through circulation delivers a naked RNA to many organs with or without the target organ specificity. Alternatively, the ts-agent is an RNA (i.e., a synthetic RNA) encapsulated by nanoparticles, which are engineered to target specific cell types, tissues, organs, cancers, tumors, or abnormal cells. Thus, systemic delivery through circulation delivers a nanoparticle-encapsulated RNA to specific cell types, tissues, organs, cancers, tumors, or abnormal cells. Alternatively, the ts-agent is an RNA packaged into a viral particle. Depending on the envelope types and other features, a virus particle targets specific cell types, tissues, organs, cancers, tumors, or abnormal cells. Thus, systemic delivery through circulation delivers an RNA packaged into a viral particle to specific cell types, tissues, organs, cancers, tumors, or abnormal cells. Alternatively, ts-agent is a temperature-sensitive virus vector. Depending on the envelope types and other features, a virus particle targets specific cell types, tissues, organs, cancers, tumors, or abnormal cells. Thus, systemic delivery through circulation delivers a temperature-sensitive virus vector to specific cell types, tissues, organs, cancers, tumors, or abnormal cells.

Targeting Delivery to the Brain and Spinal Cord Through Cerebrospinal Fluid

A patient is placed under hypothermic conditions (e.g., at 33° C.). Once the patient's core body temperature is maintained at the target temperature stably, a ts-agent is delivered directly to the patient's cerebrospinal fluids by an epidural injection. The ts-agent is delivered to the brain and spinal cord. The core body temperature of the patient continues to be maintained at the permissive temperature for the desired duration (e.g., 24 hours). While the patient's body temperature is kept at a permissive temperature for the agent (e.g., at 33° C.), the agent is functioning. When the patient's body temperature is returned to normal at 37° C., which is a non-permissive temperature of the agent, the agent stops working.

Targeting Delivery to Liver, Kidney, Skeletal Muscles, Cardiac Muscles, Pancreas, Bone Marrow, and Other Organs Through Percutaneous Injection

A patient is placed under hypothermic conditions (e.g., at 33° C.). Once the patient's core body temperature is maintained at the target temperature stably, a ts-agent is injected through the skin (percutaneously) into organs such as the liver, kidney, skeletal muscles, cardiac muscles, pancreas, or other organs using a very thin needle with the visual guidance of ultrasound or CT. The core body temperature of the patient is maintained at the permissive temperature for the desired duration (e.g., 24 hours). While the patient's body temperature is kept at the permissive temperature for the agent (e.g., at 33° C.), the agent is functioning. When the patient's body temperature is returned to normal at 37° C., which is a non-permissive temperature of the agent, the agent stops working.

Targeting Delivery to Liver, Kidney, Skeletal Muscles, Cardiac Muscles, Pancreas, Bone Marrow, and Other Organs Through Endoscopy with Injection Needle Catheter

A patient is placed under hypothermic conditions (e.g., at 33° C.). Once the patient's core body temperature is maintained at the target temperature stably, then a ts-agent is delivered directly to specific organs and tissues through endoscopic injection needle catheter. The core body temperature of the patient is maintained at the permissive temperature for the desired duration (e.g., 24 hours). While the patient's body temperature is kept at a permissive temperature for the agent (e.g., at 33° C.), the agent is functioning. When the patient's body temperature is returned to normal at 37° C., which is a non-permissive temperature of the agent, the agent stops working.

Targeting Delivery to Liver, Kidney, Skeletal Muscles, Cardiac Muscles, Pancreas, and Other Organs Through Angiocatheter

A patient is placed under hypothermic conditions (e.g., at 33° C.). Once the patient's core body temperature is maintained at the target temperature stably, then a ts-agent is delivered directly to specific organs and tissues through angiocatheter. The core body temperature of the patient is maintained at a permissive temperature for the desired duration (e.g., 24 hours). While the patient's body temperature is kept at a permissive temperature for the agent (e.g., at 33° C.), the agent is functioning. When the patient's body temperature is returned to normal at 37° C., which is a non-functional temperature of the agent, the agent stops working.

Targeting Delivery to Lung and Other Organs Through Inhalation

A patient is placed under hypothermic conditions (e.g., at 33° C.). Once the patient's core body temperature is maintained at the target temperature stably, then a ts-agent is delivered directly to the patient by inhalation. The ts-agent is delivered to lungs and other organs through via inhalation through the lungs. The core body temperature of the patient is maintained at a permissive temperature for the desired duration (e.g., 24 hours). While the patient's temperature is kept at a permissive temperature for the agent (e.g., at 33° C.), the agent is functioning. When the patient's body temperature is returned to normal at 37° C., which is a non-permissive temperature of the agent, the agent stops working.

Targeting Delivery to Bone Marrow Cells Mobilized to Spleen

A patient will receive an injection of G-CSF, plerixafor or other cytokines to mobilize bone marrow cells (including, without limitation, CD34+ cells, hematopoietic stem cells, mesenchymal stem cells, and endothelial stem cells) to the spleen of the subject. The patient is placed under hypothermic conditions (e.g., at 33° C.). Once the patient's core body temperature is maintained at the target temperature stably, then a ts-agent is delivered to the spleen via the methods described above. Subsequently, the ts-agent is delivered to bone marrow cells mobilized to the spleen. The core body temperature of the patient is maintained at a permissive temperature for the desired duration (e.g., 24 hours). While the patient's temperature is kept at a permissive temperature for the agent (e.g., at 33° C.), the agent is functioning. When the patient's body temperature is returned to normal temperature at 37° C., which is a non-permissive temperature for the agent, the agent stops working. For instance, the methods may include administering a therapeutically effective amount of a temperature-sensitive agent (e.g., a temperature-sensitive therapeutic agent) to one or more bone marrow cells (including, without limitation, CD34+ cells, hematopoietic stem cells, mesenchymal stem cells, endothelial stem cells) in the spleen.

### Example 11: Optimal Ex Vivo Contact Conditions for SeVts-ZSCAN4 on Human Mobilized Peripheral Blood CD34+ Cells

This example describes the finding that a 16-hour incubation at 33° C. ex vivo was sufficient for a temperature-sensitive Sendai Virus Vector to have effects on human CD34+ cells. This example demonstrates that a multiplicity of infection (MOI) of 1 to 25 was sufficient for the vector to infect the majority of human CD34+ cells ex vivo.

### Materials and Methods

Cell Culture

A frozen sample of human peripheral blood CD34+ cells, purified by CD34+ magnetic beads, was thawed and cultured in media supplemented with StemMACS HSC expansion cocktail, which contains a combination of recombinant human stem cell factor (SCF), Flt3-ligand, and thrombopoietin (TPO). In these culture conditions, most CD34+ cells did not divide for the first few days, as only up to two cell divisions occurred even when cells were cultured for 10 days.

Sendai Virus Vector Encoding Human ZSCAN4 Gene

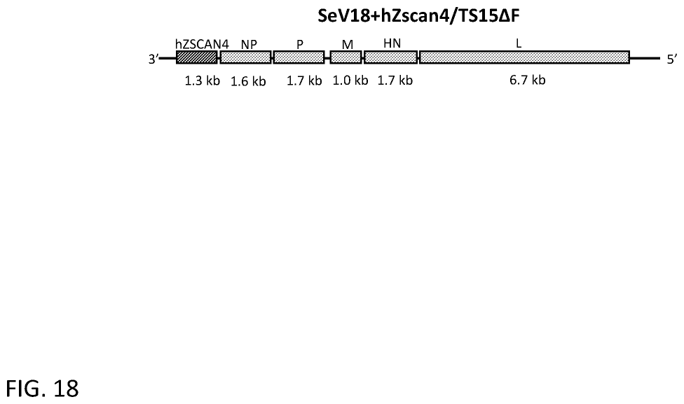

SeV18+TS15ΔF is a temperature-sensitive version of Sendai Virus vector with a TS15 backbone (Ban et al., PNAS 2011), which was custom-made by ID Pharma (Tsukuba, Japan). This vector backbone lacks the F(usion) gene, which is required to reproduce the infectious progeny virus. Thus, this vector does not transmit virus from infected cells to uninfected cells. This vector encodes two RNA polymerase genes (P and L), and three structural protein genes (NP, M and HN), and contains point mutations in the M, HN, P and L genes, which makes the vector temperature-sensitive: replicates at 33° C. (or below 35° C.), but ceases replication at 37° C. SeV18+hZSCAN4/TS15ΔF (also referred to herein as “SeVts-ZSCAN4”) is a SeV18+TS15ΔF Sendai Virus Vector encoding human ZSCAN4 gene, which was custom-made by the ID Pharma (Tsukuba, Japan). A schematic of the genome of SeV18+hZSCAN4/TS15ΔF (i.e., SeVts-ZSCAN4) is shown in FIG. 18.

Multiplicity of Infection (MOI)

Optimum multiplicity of infection (MOI) is varied among different experimental conditions. For example, not only the MOI, but also a total volume of the culture media was found to influence the infection efficiency of the Sendai virus vector. Our standard MOI=25 was determined in the following manner. First, it has previously been shown that for CD34+ cells MOI=20 results in 100% efficiency, whereas MOI=2 results in 43% (Ban et al., 2011). For mouse embryonic stem cells, MOI=10, MOI=30, MOI=100 were compared and MOI=30 showed the highest efficiency (Amano et al., 2015). For human fibroblast cells, MOI=25 showed 55.6% efficiency, whereas MOI=5, 14%, and MOI=10, 25.4% (Amano et al., 2015). Our CD34+ data showed that MOI=25 results in 53% efficiency, whereas MOI=10 results in 33% efficiency. Further studies showed that MOI=25 consistently results in CD34+ cells efficiency of 75.6±14.2% (mean±SD, n=16). In a later study, MOI=1.1 showed a 89.8% efficiency in human CD34+ cells. Thus, dependent on the experimental conditions, an MOI=1-25 is selected for SeVts-ZSCAN4 infection.

### Results

In order to determine the optimal time and conditions for ex vivo contact of CD34+ cells with SeVts-ZSCAN4, a series of incubation times at the functional temperature (33° C.) were evaluated on CD34+ cells utilizing the intended clinical CD34+ incubation protocol. CD34+ cells were plated onto 12-well plates (1×10⁵ or 5×10⁴ cells/well) and incubated with SeVts-ZSCAN4 (MOI=25) at 33° C. in 5% CO₂ for 0, 3, 6, 16, 24, 48 or 72 hours. The cells were then incubated at 37° C. in 5% CO₂ for up to 10 days. The incubation at 33° C. allows for Sendai virus infection, replication, and transgene expression, whereas raising the temperature to 37° C. inactivates the virus and turns off transgene expression. Cells were immunostained for ZSCAN4 protein expression with an anti-hZSCAN4 antibody after the prescribed 33° C. incubation. The number of human ZSCAN4 expressing cells was compared to the total number of cells identified by DAPI fluorescent staining.

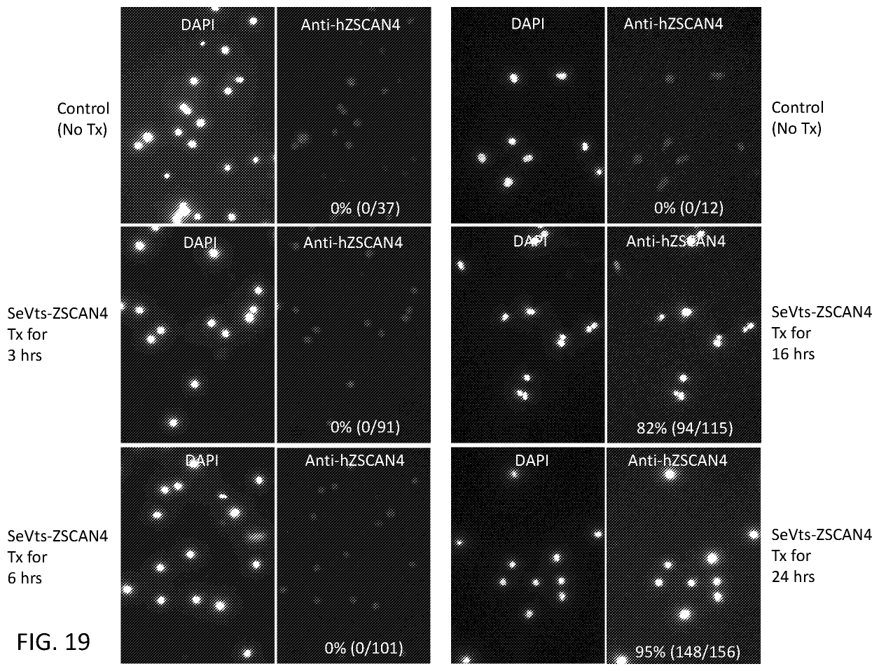

The 3 and 6 hour incubations were too short to express ZSCAN4 protein at a detectable level. However, incubations of 16 and 24 hours at 33° C. resulted in ZSCAN4 protein expression of 82% and 95% of CD34+ cells, respectively (FIG. 19). There was no further increase of the transfection efficiency and protein expression after 48 and 72 hour incubations at 33° C.

### Example 12: Kinetics of ZSCAN4 Protein in Human CD34+ Cells

This example describes the finding that a temperature shift from 33° C. to from 37° C. turned off the expression of ZSCAN4 protein, which disappeared precipitously.

### Materials and Methods

Sendai Virus Vector Encoding Human ZSCAN4 Gene

SeVts-ZSCAN4 (also called SeV18+hZSCAN4/TS15ΔF) expresses human ZSCAN4 in a temperature-sensitive manner (FIG. 18).

### Results

To determine the time of exposure to ZSCAN4 protein, the kinetics of ZSCAN4 protein expression in CD34+ cells was examined. To closely mimic the proposed clinical trial situation CD34+ cells, isolated by mobilization of peripheral HSCs, were obtained from Hemacare, Inc.

CD34+ cells were left untreated or contacted with SeVts-ZSCAN4 at 33° C. for 24 hours and further incubated at 37° C. for 9 days. Cells were sampled at day 1, 3, 7 and 10, and immunostained with antibodies against CD34 and ZSCAN4.

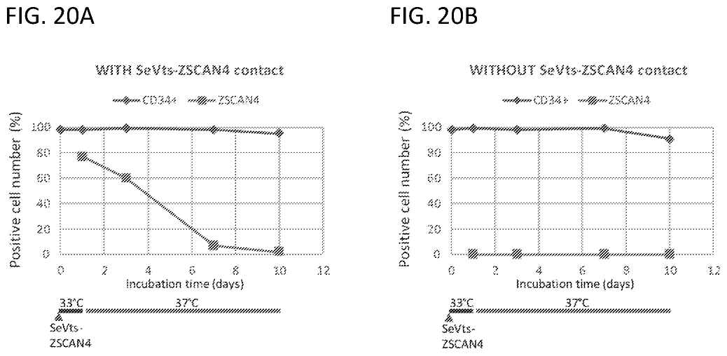

During the 10-day incubation period, nearly 100% of cells retained their CD34 marker, which indicated that contact with SeVts-ZSCAN4 did not change the nature of CD34+ cells in terms of the fraction of CD34+ cells and CD34 marker expression (FIG. 20A, B). Based on the immunostaining with ZSCAN4 on Day 1, the contact with SeVts-ZSCAN4 (MOI=25) exposed 77% of CD34+ cells to ZSCAN4 protein (FIG. 20A). As expected, once the temperature was shifted to 37° C., cells with ZSCAN4 proteins rather quickly decreased: there were only 7% ZSCAN4-positive cells by day 7 and 2% by day 10. By contrast, the control experiment shows that without SeVts-ZSCAN4 contact, no ZSCAN4-positive cells were present, but nearly 100% of cells remained CD34+. The rapid decline of ZSCAN4 protein after switching to the non-permissive temperature, 37° C., was not simply caused by cell division, because the number of cells increased only by 3.5-fold (fewer than two cell divisions on average) over 10 days. During the same time, the number of control cells (no SeVts-ZSCAN4 contact) increased by 6.1-fold.

### Example 13: Effect of SeVts-ZSCAN4 on Telomere Length of Human CD34+ Cells

This example describes the finding that transient expression of human ZSCAN4 using a temperature-sensitive viral vector increased the length of telomeres in human CD34+ cells.

### Materials and Methods

Sendai Virus Vector Encoding Human ZSCAN4 Gene

SeVts-ZSCAN4 (also called SeV18+hZSCAN4/TS15ΔF) expresses human ZSCAN4 in a temperature-sensitive manner (FIG. 18).

### Results

ZSCAN4 has been shown to localize to the telomere, upregulate meiosis-specific homologous recombination genes, and extend telomeres through telomere recombination (independent of telomerase activity) in mouse embryonic stem (ES) cells (Zalzman et al., 2010; Amano et al., 2013). To evaluate this potential in human hematopoietic stem cells, human peripheral blood CD34+ cells were contacted ex vivo with SeVts-ZSCAN4 and incubated at 33° C. CD34+ cells were treated with SeVts-ZSCAN4 for 16, 24, 48 and 72 hours at 33° C., and were then cultured at 37° C. for 10 days for use in the telomere assay. The length of telomeres was measured by the quantitative real-time PCR method using a telomere-specific primer (T) and a single copy gene-specific primer set (S) as described (Cawthon 2002). Relative telomere length was calculated as a T/S ratio and further normalized by the T/S ratio of a control sample (non-treated control).

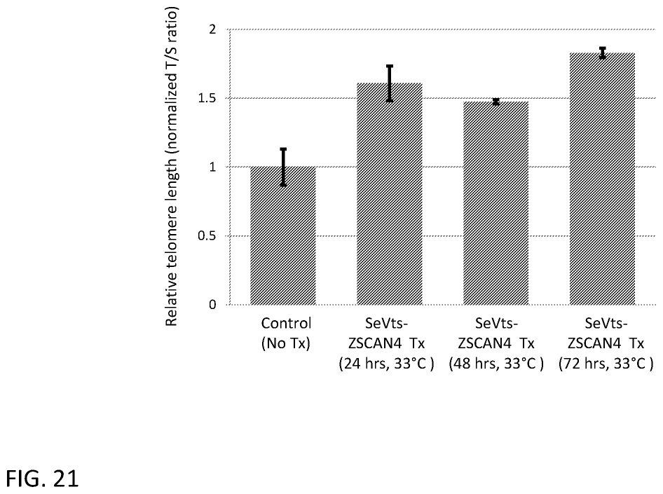

Compared to the non-treated cells, the 24-hour incubation at 33° C. extended telomeres approximately 1.5-fold (FIG. 21). The incubations ≥24 hours did not extend telomeres further; thus, 24-hour incubation at permissive temperature (i.e., 33° C.) was sufficient to extend telomeres of human CD34+ cells.

### Example 14: Effect of SeVts-ZSCAN4 on Telomere Length of Human Blood Cells Engrafted in Immune Compromised Mice

This example describes a procedure to evaluate the safety of administering CD34+ cells treated with a temperature-sensitive Sendai Virus Vector, expressing the human ZSCAN4 gene, to a subject, and the efficacy of engraftment of the cells.

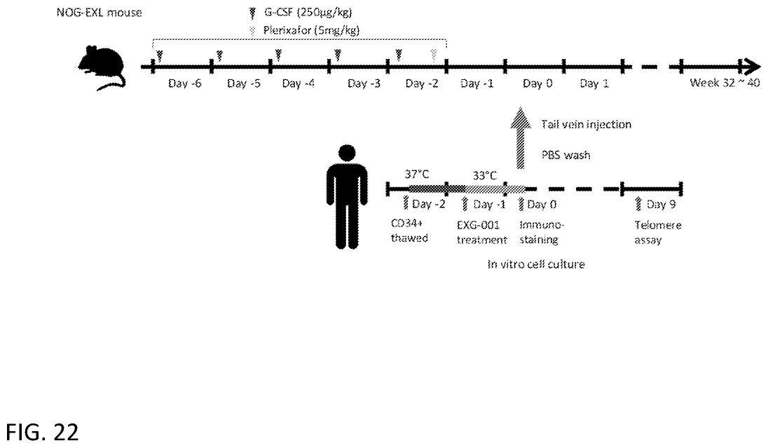

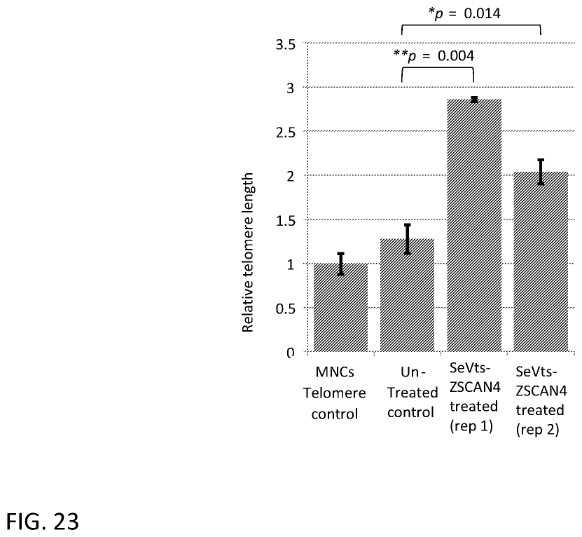

SeVts-ZSCAN4 (also called SeV18+hZSCAN4/TS15ΔF) expresses human ZSCAN4 in a temperature-sensitive manner (FIG. 18). Human CD34+ cells were contacted with SeVts-ZSCAN4 (MOI=25) in culture at a permissive temperature of 33° C. for 24 hours, in a manner suitable for intended clinical use. The cells were then washed to remove free SeVts-ZSCAN4 and re-suspended in saline (test materials) (FIG. 22). Aliquots of the test materials were further cultured in vitro for 10 days and subjected to the telomere length assay by qPCR (FIG. 22). MNC is the mononuclear cell standard used for telomere length. Ratio of telomere length (T/S ratio) of samples relative to that of MNCs was presented as relative telomere length. The telomeres of CD34+ treated with SeVts-ZSCAN4 for 24 hours were statistically significantly longer than those of CD34+ untreated cells (FIG. 23). Therefore, SeVts-ZSCAN4 treatment for 24 hours at the permissive temperature (i.e., 33° C.) was able to extend telomeres of human CD34+ cells in vitro.

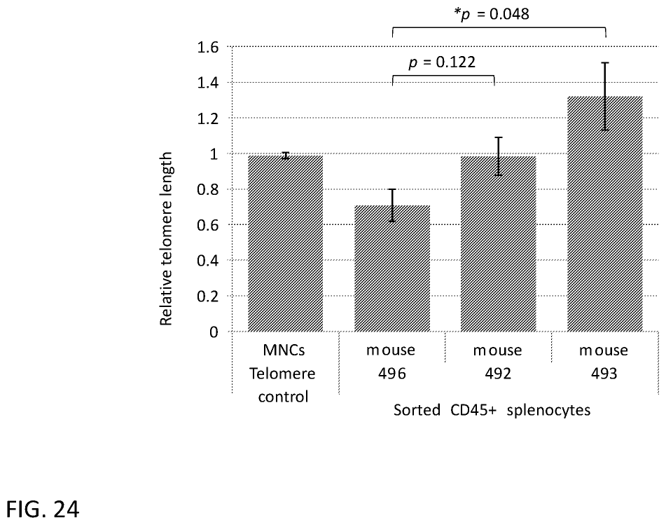

To closely model the intended clinical trial, severely immunodeficient NOG-EXL mice (Taconic) were treated with G-CSF and Plerixafor (FIG. 22). The study used NOG-EXL mice without irradiation (i.e., bone marrow ablation). Also, unlike typical engraftment studies which use more potent cord blood CD34+ cells, the study used G-CSF-mobilized peripheral blood CD34+ cells from healthy donors. The NOG-EXL mice were intravenously administered either CD34+ untreated cells or CD34+ treated with SeVts-ZSCAN4 (test materials) at dosages of 2×10⁷ cells/kg on Day 1 (FIG. 22). The dose is approximately 10-fold greater than the dose intended for humans. No SeVts-ZSCAN4-related adverse event was observed among the NOG-EXL mice received CD34+ cells treated with SeVts-ZSCAN4. Furthermore, after 38 weeks of CD34+ cell injection, two mice (#492 and #493) received CD34+ treated with SeVts-ZSCAN4 and one mouse (#496) received CD34+ cells untreated (control) were sacrificed to examine the engraftment cells derived from human CD34+ cells. Splenocytes isolated from these mice were FACS-sorted by human CD45+ pan-hematopoietic marker and used to do qPCR-based telomere assays. As shown in FIG. 24, telomeres of human cells engrafted in the mice received CD34+ cells treated with SeVts-ZSCAN4 were longer than those in the mouse received CD34+ cells only (control). These data indicated that SeVts-ZSCAN4-treated human CD34+ cells can engraft in mouse bone marrow and participated in the normal hematopoiesis. Also, once the telomeres are extended by the treatment of SeVts-ZSCAN4, the telomeres of these cells are longer even after the engraftment and cell differentiation. The study also indicates the safety of SeVts-ZSCAN4 treatment.

### Example 15: Evaluation of SeVts-ZSCAN4 in Human Patients with Telomere Biology Disorders and Bone Marrow Failure

Telomere biology disorders with bone marrow failure including dyskeratosis congenita have a poor prognosis and a high mortality rate. Currently the hematopoietic stem cell transplantation is the only curative treatment, which can alleviate the hematologic manifestations of the condition. However, its use can be challenging with difficulties in finding well-matched donors and toxicities related to myeloablation (chemotherapy and radiation) and immune complications. This example describes evaluation of the safety and tolerability of administering CD34+ cells contacted ex vivo with a temperature-sensitive Sendai Virus Vector encoding human ZSCAN4, to a human patient in need thereof, and the efficacy of engraftment of the cells.

Therapeutic Temperature-Sensitive Agent. SeVts-ZSCAN4 (also called SeV18+hZSCAN4/TS15ΔF) expresses human ZSCAN4 in a temperature-sensitive manner (FIG. 18). As used in this example, the study drug product is a pharmaceutical composition comprising a sterile, electrolyte-containing, isotonic aqueous solution in which autologous CD34+ cells contacted ex vivo with SeVts-ZSCAN4 are suspended. PLASMA-LYTE multiple electrolytes injection solution marketed by Baxter International Inc. (Deerfield, Ill.) is a suitable solution for resuspension of virus-contacted CD34+ cells.

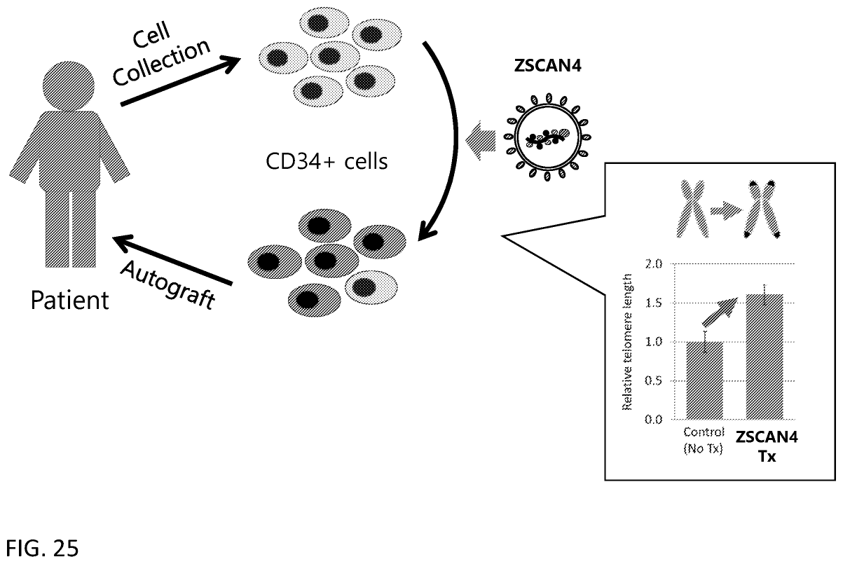

Rationale. Autologous CD34+ cells contacted ex vivo with SeVts-ZSCAN4 have been shown to extend telomeres in human CD34+ cells in vitro and in vivo nonclinical studies. This treatment is not required a well-matched donor when a patient's own cells can be used. Contact with SeVts-ZSCAN4 results in the transient production of human ZSCAN4 protein in patient's own (autologous) CD34+ cells, which restores their function by extending their abnormally short telomeres ex vivo. After dosing, contacted CD34+ cells are engrafted and subsequently proliferate in patient's bone marrow resulting in the production of blood cells. In this way, bone marrow failure of the patient is effectively treated (FIG. 25).

Patients. The study population includes initially adult men and women, but will extend to pediatric patients. Inclusion criteria include mild or moderate bone marrow failure and diagnosis of a telomere biology disorder. Mild or moderate bone marrow failure is defined by one or both of: 1) absolute neutrophil count (ANC) in peripheral blood of 0.5-1.5×10\hat{ }9/L; or platelets 20-100×10\hat{ }9/L; or hemoglobin <10 g/dL; and 2) hypocellular bone marrow for age. Diagnosis of a telomere biology disorder is defined by one of the following: 1) age-adjusted mean telomere length<1 percentile in any of peripheral blood lymphocytes (PBL), B-cells, or naïve T-cells; or 2) a pathogenic mutation in DKC1, TERC, TERT, NOP10, NHP2, TINF2, CTC1, PARN, RTEL1, ACD, USB1, or WRAP53. Exclusion criteria include one or more of the following: receiving chemotherapy for cancer; clonal cytogenetic abnormalities associated with myelodysplastic syndrome or acute myeloid leukemia on bone marrow examination; uncontrolled bacterial, viral or fungal infections; prior allogeneic marrow or stem cell transplantation; subjects who are not eligible for G-CSF and plerixafor; subjects who are not eligible for the apheresis; subjects currently taking or have taken danazol and androgens within 60 days prior to start of the study.

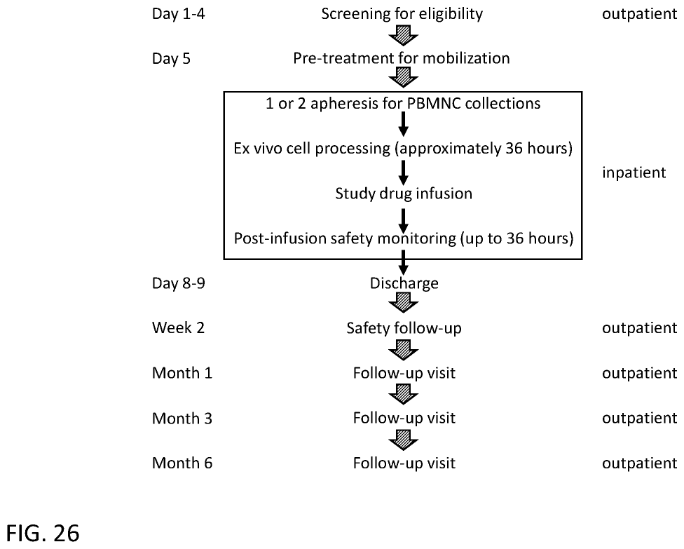

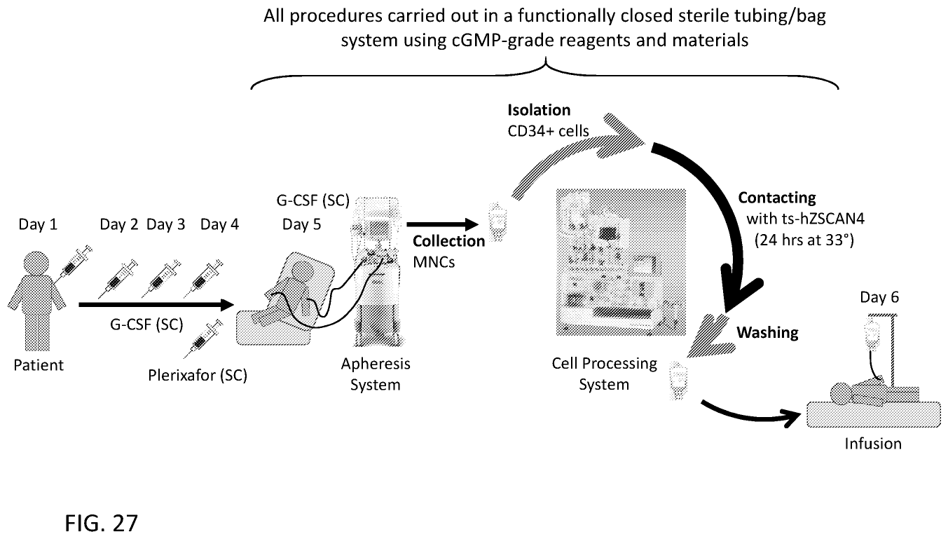

Procedures. In brief, the study involves: 1) mobilization of hematopoietic stem cells into the blood stream and collection of mononuclear cells (MNCs) by apheresis; 2) ex vivo cell processing; and 3) infusion of processed cells. A flow chart of the study design in shown in FIG. 26 and a schematic is shown in FIG. 27.

Mobilization and Apheresis.

Days 1 to 3: All eligible subjects receive a daily granulocyte-colony stimulating factor (G-CSF) injection (10 μg/kg SC).
Day 4: After G-CSF injection (10 μg/kg SC), a blood sample is collected and CD34+ cell counts are determined. Subjects who have <5 cells/μL of CD34+ cells are withdrawn from the study. Subjects who have ≥5 cells/μL of CD34+ cells are hospitalized, and plerixafor (20 mg fixed dose or 0.24 mg/kg SC) is administered approximately 11 hours prior to apheresis. Plerixafor (1,4-Bis((1,4,8,11tetraazacyclotetradecan-1-yl)methyl)benzene, CAS No. 155148-31-5), such as MOZOBIL marketed by Genzyme Corporation (Cambridge, Mass.), is a hematopoietic stem cell mobilizer.
Day 5: G-CSF (10 μg/kg SC) is administered and a first apheresis is initiated to collect MNCs. After apheresis, subjects are assessed for the ability to tolerate a second apheresis. Plerixafor (20 mg fixed dose or 0.24 mg/kg SC) is administered to subjects deemed to be able to tolerate a second apheresis approximately 11 hours prior to a second apheresis. Subjects unable to tolerate a second apheresis and who have <2.0×10\hat{ }6/kg CD34+ cells are withdrawn from the study and all collected cells are infused back into the subject. Subjects unable to tolerate a second apheresis and who have ≥2.0×10\hat{ }6/kg CD34+ cells continue on the study.
Day 6: Subjects able to tolerate a second apheresis receive G-CSF (10 μg/kg SC) prior to initiation of a second apheresis to collect additional MNCs. Post apheresis, a complete blood count (CBC) is obtained and the subject is given a red blood cell or platelet transfusion, if needed, to keep hemoglobin levels>10.5 g/dl and platelets >100K. Subjects who have undergone a second apheresis and who have <2.0×10\hat{ }6/kg CD34+ cells (total of first and second apheresis) are withdrawn from the study and all collected cells are infused back into the subject. Subjects who have undergone a second apheresis and who have ≥2.0×10\hat{ }6/kg CD34+ cells continue on the study.

Ex Vivo Cell Processing. CD34+ cells are isolated from MNCs collected by apheresis using a CLINIMACS PRODIGY automated cell processing system marketed by Miltenyi Biotec (Germany) under good manufacturing practices. CD34+ cells are suspended in in GMP-grade HSC-Brew GMP Medium and cytokines (equivalent to StemMacs media), contacted with SeVts-ZSCAN4 at a MOI=1˜MOI=25 (dependent on collected CD34+ cell number), and cultured for 1 hr at the permissive temperature of 33° C. Additional HSC-Brew GMP Medium is added to the virus-contacted CD34+ cells, which are cultured for a further 23 hours at the permissive temperature of 33° C. After incubation the virus-contacted CD34+ cells are washed three times with HSC-Brew GMP Medium to remove free SeVts-ZSCAN4 and resuspended in 100 mL of a sterile, PLASMA-LYTE to produce the study drug product.

Infusion. Subjects receive a single intravenous infusion of the study drug product at a dose of 2.0-8.0×10\hat{ }6/kg CD34+ cells suspended in 100 mL of PLASMA-LYTE multiple electrolytes for injection marketed by Baxter Healthcare Corporation (Deerfield, Ill.) or other sterile electrolyte-containing, isotonic aqueous solution. The cells are delivered at a 3.3 mL/min infusion rate over the course of 30 minutes. Study drug product infusion takes place about 32 hours after the first apheresis.

Safety assessments are done until 24, 36 or 48 hours post-infusion and include but are not limited to assessment of vital signs (body temperature, pulse, respiration rate and blood pressure), weight, electrocardiogram, clinical laboratory tests (hematology, blood chemistry and urinalysis), adverse events, plasma cytokine levels and immunogenicity of the study drug product. Plasma cytokine(s) that are measured include one or more of GM-CSF, IFN-gamma, IL-1beta, IL-2, IL-4, IL-5, IL-6, IL-8 and TNF-alpha). Immunogenicity of the study drug is assessed by measuring Sendai-virus vector-reactive and human SCAN4-reactive antibodies in blood samples obtained from the subject.

Exploratory Endpoints. Increase in telomere length in any of the following: lymphocytes, granulocytes, B-cells, naïve T-cells, memory T-cells, and NK cells in peripheral blood, and improvement of blood counts (neutrophils, platelets, or hemoglobin). Telomere length is measured by Flow FISH.

### Example 16: Expression of Human ZSCAN4 Protein In Vivo

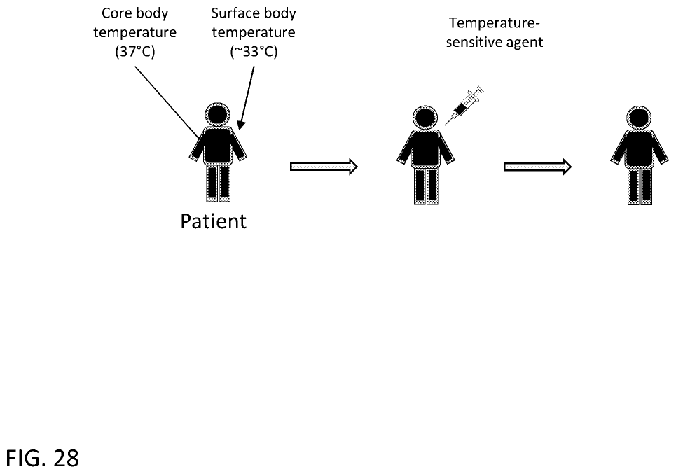

Temperature-sensitive agents (ts-agents) such as srRNAs or Sendai virus vectors, are functional at a permissive temperature (e.g., about 31-34° C.), but non-functional at a non-permissive temperature (e.g., 37° C.). While the core body temperature of a human subject is about 37° C., the surface body temperature of a human subject is about 31-34° C. Thus, ts-agents administered to cells at or near the surface of a body of a human patient (e.g., intradermally, subcutaneously, or intramuscularly) are functional without lowering the core body temperature of the human patient (FIG. 28). No further action is required.

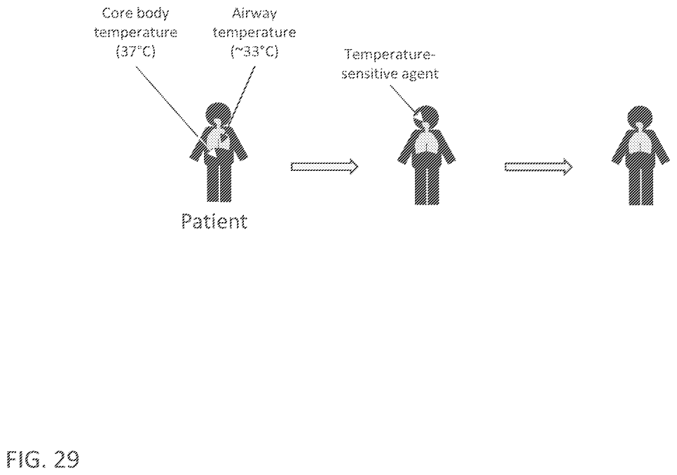

Similarly, the temperature of the nasal cavity and upper trachea of a human subject is about 32° C., and the temperature of the subsegmental bronchi of a human subject is about 35° C. (McFadden et al., 1985). As such, ts-agents administered intranasally to cells of the upper respiratory tract (nasal cavity, pharnyx, and/or larnyx) and/or upper trachea of a human patient are functional without lowering the core body temperature of the human patient (FIG. 29). Intranasal administration may be done by insufflation, inhalation or instillation. No further action is required.

Alternatively, the ts-agent administered to cells at or near the surface of a body of a human patient (e.g., intradermally, subcutaneously, or intramuscularly) can subsequently be rendered non-functional by raising the surface body temperature of the human patient, for instance by application of a heat patch or heating pad to the treated area of the patient's skin, soaking in a warm bath, or sitting in a hot sauna. This therapeutic procedure is very safe in that the ts-agent is only functional in the intended area and is non-functional in other areas of a patient's body. Similarly, the ts-agent administered intranasally to cells of the upper respiratory tract (nasal cavity, pharnyx, and/or larnyx) and/or upper trachea of a human patient can be rendered non-functional by placing the human patient in an environment with a non-permissive temperature (e.g., ≥37° C.).

For instance, a coding region of human ZSCAN4 is introduced into srRNA1ts2 or SeV18/TS15ΔF as described above for expression of human ZSCAN4 at or near the surface of human patient's body. The construction of srRNA1ts2 is described above in Example 3. In brief, srRNA1ts2 comprises a Venezuelan equine encephalitis virus (VEEV) replicon lacking a VEEV structural protein coding region. The VEEV replicon comprises a VEEV nonstructural protein coding region with an insertion of 15-18 nucleotides resulting in expression of a nonstructural Protein 2 (nsP2=helicase proteinase) comprising 5 or 6 additional amino acids (SEQ ID NO:44=TGAAA) between beta sheet 5 and beta sheet 6 of the nsP2. The additional amino acids result in temperature-sensitivity of the srRNA.

RNA of a srRNA1ts2 vector can be transcribed in vitro using T7 RNA polymerase without the use of materials of animal or human origin. In this way, ts-agents employing srRNA1ts2 vectors are easily adapted to production using current good manufacturing practice. The RNAs are transfected into cells of a subject's dermal tissue. A suitable method for transfection is by patch electroporation of naked RNAs. Alternatively, a microneedle is used to transfect RNAs intradermally. For instance, a dissolvable microneedle made with hyaluronic acid or a chitosan-hyaluronic acid complex is used to transfect RNAs intradermally.

What is claimed:

1. A method of treating a disease of blood or a blood-forming organ, comprising:
i) mobilizing hematopoietic stem cells from bone marrow to peripheral blood of a human subject suffering from the disease;
ii) isolating CD34+ cells from a sample of peripheral blood mononuclear cells obtained from the subject;
iii) incubating the isolated CD34+ cells at a temperature of 33° C. 0.5° C.;
iv) contacting the incubated CD34+ cells with a temperature-sensitive Sendai viral vector comprising a heterologous nucleic acid comprising a coding region of human ZSCAN4;
v) maintaining the contacted CD34+ cells at a permissive temperature of 33° C.±0.5° C. for a period of at least about 12-72 hours, wherein replication and transcription of the temperature-sensitive Sendai viral vector occurs at the permissive temperature leading to increased expression of human ZSCAN4; and
vi) infusing the contacted CD34+ cells into the subject under conditions suitable for engrafting the cells to treat the disease.

2. A method of treating a disease of blood or a blood-forming organ, comprising:
i) mobilizing hematopoietic stem cells from bone marrow cell to peripheral blood of a human subject suffering from the disease;
ii) isolating CD34+ cells from a sample of peripheral blood mononuclear cells obtained from the subject;
iii) contacting the isolated CD34+ cells with a temperature-sensitive Sendai viral vector comprising a heterologous nucleic acid comprising a coding region of human ZSCAN4;
iv) incubating the contacted CD34+ cells at a permissive temperature of 33° C.±0.5° C. for a period of at least about 12-72 hours, wherein replication and transcription of the temperature-sensitive Sendai viral vector occurs at the permissive temperature leading to increased expression of human ZSCAN4; and
v) infusing the contacted CD34+ cells into the subject under conditions suitable for engrafting the cells to treat the disease.

3. The method of claim 1, further comprising after step v) incubating the contacted CD34+ cells at a non-permissive temperature of 37° C.±0.5° C. prior to infusing the contacted CD34+ cells into the subject, wherein replication and transcription of the temperature-sensitive Sendai viral vector and expression of human ZSCAN4 ceases at the non-permissive temperature.

4. The method of claim 2, further comprising after step iv) incubating the contacted CD34+ cells at a non-permissive temperature of 37° C.±0.5° C. prior to infusing the contacted CD34+ cells into the subject, wherein replication and transcription of the temperature-sensitive Sendai viral vector and expression of human ZSCAN4 ceases at the non-permissive temperature.

5. The method of claim 3 or claim 4, wherein the contacted CD34+ cells are incubated at the non-permissive temperature of 37° C.±0.5° C. for about 30 minutes to about 10 days, optionally for about 30-180 minutes.

6. The method of claim 5, wherein the hematopoietic stem cells are mobilized by administration of one or both of granulocyte-colony stimulation factor and plerixafor to the subject.

7. The method of claim 6, wherein the peripheral blood mononuclear cells are obtained from the subject by apheresis.

8. The method of claim 7, wherein the CD34+ cells are isolated from the peripheral blood mononuclear cells by positive selection using an anti-CD34 antibody and magnetic beads.

9. The method of claim 8, wherein the contacted CD34+ cells are washed and resuspended in a sterile, isotonic aqueous solution prior to infusion.

10. The method of claim 9, wherein the contacted CD34+ cells are intravenously infused at a dose of about 1.0×10\hat{ }5 cells/kg to about 1.0×10\hat{ }7 cells/kg, optionally about 2.0-8.0×10\hat{ }6 cells/kg.

11. A method of treating a disease of blood or a blood-forming organ, comprising:
i) administering to a human subject suffering from the disease a temperature-sensitive Sendai viral vector comprising a heterologous nucleic acid comprising a coding region of human ZSCAN4;
ii) lowering the subject's core body temperature to a permissive temperature of 33° C.±0.5° C.;
iii) maintaining the subject's core body temperature at the permissive temperature for a period of from about 12 hours to about 7 days, or for about 12-72 hours, wherein replication and transcription of the temperature-sensitive Sendai viral vector occurs at the permissive temperature leading to increased expression of human ZSCAN4; and
iv) allowing the subject's core body temperature to return to a normal, non-permissive temperature of 37° C.±0.5° C., wherein replication and transcription of the temperature-sensitive Sendai viral vector and expression of human ZSCAN4 ceases at the non-permissive temperature.

12. A method of treating a disease of blood or a blood-forming organ, comprising:
i) lowering the core body temperature of a subject suffering from the disease to a permissive temperature of 33° C.±0.5° C.;
ii) administering to the subject a temperature-sensitive Sendai viral vector comprising a heterologous nucleic acid comprising a coding region of human ZSCAN4;
iii) maintaining the subject's core body temperature at the permissive temperature for a period of from about 12 hours to about 7 days, or for about 12-72 hours, wherein replication and transcription of the temperature-sensitive Sendai viral vector occurs at the permissive temperature leading to increased expression of human ZSCAN4; and
iv) allowing the subject's core body temperature to return to a normal, non-permissive temperature of 37° C.±0.5° C., wherein replication and transcription of the temperature-sensitive Sendai viral vector and expression of human ZSCAN4 ceases at the non-permissive temperature.

13. The method of claim 11 or claim 12, wherein the subject's core body temperature is lowered using a targeted temperature management (TTM) procedure, wherein the TTM procedure comprises application to the subject of one of the group consisting of a cooling catheter, a cooling blanket, and ice.

14. The method of claim 13, wherein the human subject is diagnosed with bone marrow failure prior to treatment, optionally wherein the bone marrow failure comprises one or more of neutropenia, thrombocytopenia, and anemia.

15. The method of claim 14, wherein the subject does not have cancer.

16. The method of claim 15, wherein the disease is a telomere biology disorder.

17. The method of claim 16, where the telomere biology disorder is selected from the group consisting of dyskeratosis congenita, Hoyeraal-Hreidarsson syndrome, Revesz syndrome, Coats plus syndrome, idiopathic pulmonary fibrosis, and cirrhosis.

18. The method of claim 16, wherein the telomere biology disorder is defined by one or both of:
i) age-adjusted mean telomere length of less than 1 percentile in one or more of peripheral blood lymphocytes, B-cells, and naïve T-cells; and
ii) a pathogenic mutation in a gene selected from the group consisting of DKC1, TERC, TERT, NOP10, NHP2, TINF2, CTC1, PARN, RTEL1, ACD, USB1, and WRAP53.

19. The method of claim 13, wherein the disease is a bone marrow failure syndrome.

20. The method of claim 19, wherein the bone marrow failure syndrome is selected from the group consisting of Fanconi anemia, amegakaryocytic thrombocytopenia, aplastic anemia, Diamond Blackfan anemia, paroxysmal nocturnal hemoglobinuria, Pearson syndrome, Shwachman Diamond syndrome, and myelodysplastic syndrome.

21. The method of claim 14, wherein the disease is associated with a karyotype abnormality.

22. A temperature-sensitive agent, wherein the agent is a temperature-sensitive viral vector or a temperature-sensitive self-replicating RNA comprising a heterologous nucleic acid comprising a coding region of human ZSCAN4, and a nonstructural protein coding region with an insertion of 12-18 nucleotides, wherein the insertion results in expression of a nonstructural Protein 2 (nsP2=helicase proteinase) comprising from 4 to 6 additional amino acids between beta sheet 5 and beta sheet 6 of the nsP2, optionally wherein the additional amino acids result in temperature-sensitivity of the viral vector or the self-replicating RNA.

23. The temperature-sensitive agent of claim 22, wherein the additional amino acids comprise one sequence selected from the group consisting of SEQ ID NO:43 (GCGRT), SEQ ID NO:44 (TGAAA), and SEQ ID NO:45 (LRPHP).

24. The temperature-sensitive agent of claim 22, wherein the additional amino acids comprise the sequence of SEQ ID NO:44 (TGAAA).

25. The temperature-sensitive agent of claim 24, wherein the amino acid sequence of the NsP2 comprises one sequence selected from the group consisting of SEQ ID NOs:29-36.

26. The temperature-sensitive agent of claim 22, wherein the agent is a temperature-sensitive Alphavirus vector.

27. The temperature-sensitive agent of claim 22, wherein the agent is a temperature-sensitive self-replicating RNA comprising an Alphavirus replicon lacking a viral structural protein coding region.

28. The temperature-sensitive agent of claim 26 or claim 27, wherein the Alphavirus is selected from the group consisting of a Venezuelan equine encephalitis virus, a Sindbis virus, and a Semliki Forrest virus.

29. The temperature-sensitive agent of claim 26 or claim 27, wherein the Alphavirus is a Venezuelan equine encephalitis virus.

30. A method for transiently inducing a temperature-sensitive activity of a temperature-sensitive agent (ts-agent) in a subject, wherein the ts-agent is a temperature-sensitive viral vector or a temperature-sensitive self-replicating RNA comprising a heterologous nucleic acid comprising a coding region of human ZSCAN4, wherein one or more cells at or near the surface of the subject's body comprise the ts-agent, wherein the temperature-sensitive activity of the ts-agent comprises expression of human ZSCAN4 at a permissive temperature, and wherein the permissive temperature is the surface body temperature of the subject, comprising:
i) maintaining the surface body temperature of the subject at the permissive temperature for a period of time sufficient for the temperature-sensitive activity to induce an effect in the subject; and
ii) increasing the surface body temperature of the subject to a non-permissive temperature for a period of time sufficient for the temperature-sensitive activity to cease in the subject.

31. A method for transiently inducing a temperature-sensitive activity of a temperature-sensitive agent (ts-agent) in a subject, wherein the ts-agent is a temperature-sensitive viral vector or a temperature-sensitive self-replicating RNA comprising a heterologous nucleic acid comprising a coding region of human ZSCAN4, wherein the temperature-sensitive activity of the ts-agent comprises expression of human ZSCAN4 at a permissive temperature, and wherein the permissive temperature is the surface body temperature of the subject, comprising:
i) administering the ts-agent to one or more cells at or near the surface of the subject's body; and
ii) maintaining the surface body temperature of the subject at the permissive temperature for a period of time sufficient for the temperature-sensitive activity to induce an effect in the subject.

32. The method of claim 31, further comprising iii) increasing the surface body temperature of the subject to a non-permissive temperature for a period of time sufficient for the temperature-sensitive activity to cease in the subject.

33. The method of claim 31, wherein the temperature-sensitive agent is administered intradermally or subcutaneously.

34. The method of claim 31, wherein the temperature-sensitive agent is administered intramuscularly.

35. The method of any one of claims 30-34, wherein the permissive temperature is from 30° C. to 36° C., or 31° C. to 35° C., or 32° C. to 34° C., or 33° C.±0.5° C., and the non-permissive temperature is 37° C.±0.5° C.

36. The method of claim 35, wherein the effect of expression of human ZSCAN4 is prophylactic effect or a therapeutic effect.

37. The method of claim 35, wherein the ts-agent is a temperature-sensitive viral vector and the temperature-sensitive activity further comprises replication and transcription of the temperature-sensitive viral vector.

38. The method of claim 37, wherein the temperature-sensitive viral vector is selected from the group consisting of a Sendai virus, an Adeno virus, an Adeno-associated virus, a Retrovirus, and an Alphavirus.

39. The method of claim 37, wherein the temperature-sensitive viral vector is an Alphavirus, optionally wherein the Alphavirus is selected from the group consisting of a Venezuelan equine encephalitis virus, a Sindbis virus, and a Semliki Forrest virus.

40. The method of claim 37, wherein the temperature-sensitive viral vector is a Sendai virus.

41. The method of claim 35, wherein the ts-agent is a temperature-sensitive self-replicating RNA and the temperature-sensitive activity further comprises one or both of replication and transcription of the temperature-sensitive self-replicating RNA.

42. The method of claim 41, wherein the self-replicating RNA comprises an Alphavirus replicon lacking an Alphavirus viral structural protein coding region.

43. The method of claim 42, wherein the Alphavirus is selected from the group consisting of a Venezuelan equine encephalitis virus, a Sindbis virus, and a Semliki Forrest virus.

44. The method of claim 42, wherein the Alphavirus is a Venezuelan equine encephalitis virus.

45. The method of claim 36, wherein the period of time sufficient for the temperature-sensitive activity to produce an effect ranges from about 12 hours to about 12 weeks, optionally wherein the period of time is from 1 to 7 days.

46. The method of claim 36, wherein the period of time sufficient to induce an effect in the subject is from about 12 hours to about 7 days, optionally wherein the period of time is from about 12 hours to about 72 hours.

47. The method of claim 36, wherein the subject is a mammalian subject, optionally wherein the subject is a human.

48. The method of any one of claims 1-47, wherein the amino acid sequence of human ZSCAN4 comprises SEQ ID NO:38 or is at least 95% identical to SEQ ID NO:38.

49. The method of any one of claims 1-47, wherein the amino acid sequence of human ZSCAN4 comprises one of the group consisting of SEQ ID NO:39, SEQ ID NO:40, SEQ ID NO:41, and SEQ ID NO:42, or is at least 95% identical to one of the group consisting of SEQ ID NO:39, SEQ ID NO:40, SEQ ID NO:41, and SEQ ID NO:42.

50. A method for transiently inducing a temperature-sensitive activity of a temperature-sensitive agent, comprising:
i) incubating one or more cells comprising the temperature-sensitive agent at a permissive temperature to induce the temperature-sensitive activity for a period of time sufficient for the temperature-sensitive activity to produce an effect in the one or more cells; and
ii) incubating the one or more cells at a non-permissive temperature, wherein the non-permissive temperature reduces the temperature-sensitive activity of the temperature-sensitive agent,
wherein the temperature-sensitive agent comprises a therapeutic agent comprising a human ZSCAN4 protein, or a nucleic acid comprising a coding region of human ZSCAN4, and the effect comprises a therapeutic effect.

51. The method of claim 50, further comprising before step i): contacting the one or more cells with the temperature-sensitive agent.

52. The method of claim 51, wherein the one or more cells are at the permissive temperature when contacted with the temperature-sensitive agent.

53. The method of claim 50, further comprising administering the one or more cells to a subject in need of the therapeutic effect.

54. The method of claim 50, wherein incubating the one or more cells at a non-permissive temperature comprises administering the one or more cells to a subject in need of the therapeutic effect, wherein the subject's body temperature is the non-permissive temperature.

55. The method of claim 54, wherein the one or more cells are further incubated at the non-permissive temperature prior to administering the one or more cells to the subject.

56. The method of claim 50, wherein the one or more cells were isolated from the subject before contacting the one or more cells with the temperature-sensitive agent.

57. The method of claim 50, wherein the therapeutic effect comprises increasing telomere length of the one or more cells.

58. The method of claim 50, wherein the one or more cells are mammalian cells.

59. The method of claim 54, wherein the subject is a human subject.

60. A method for transiently inducing a temperature-sensitive activity of a temperature-sensitive agent in a human subject, wherein one or more cells of the subject comprise the temperature-sensitive agent, wherein the temperature-sensitive activity of the temperature-sensitive agent is induced at a permissive temperature, and wherein the permissive temperature is lower than the body temperature of the subject, comprising:
i) lowering the body temperature of the subject to the permissive temperature;
ii) maintaining said lowered body temperature for a period of time sufficient for the temperature-sensitive activity to induce an effect in the subject; and
iii) raising the body temperature of the subject back to normal body temperature,
wherein the temperature-sensitive agent comprises a therapeutic agent comprising a human ZSCAN4 protein, or a nucleic acid comprising a coding region of human ZSCAN4, and the effect is a therapeutic effect.

61. A method for transiently inducing a temperature-sensitive activity of a temperature-sensitive agent in a human subject, wherein the temperature-sensitive activity of the temperature-sensitive agent is induced at a permissive temperature, and wherein the permissive temperature is lower than the body temperature of the subject, comprising:
i) lowering the body temperature of the subject to the permissive temperature;
ii) administering the temperature-sensitive agent to one or more cells of the subject;
iii) maintaining said lowered body temperature for a period of time sufficient for the temperature-sensitive activity to induce an effect in the subject; and
iv) raising the body temperature of the subject back to normal body temperature,
wherein step (i) is performed before, after, or simultaneously with step (ii), wherein the temperature-sensitive agent comprises a therapeutic agent comprising a human ZSCAN4 protein, or a nucleic acid comprising a coding region of human ZSCAN4, and the effect is a therapeutic effect.

62. The method of claim 61, wherein the temperature-sensitive agent is administered systemically.

63. The method of claim 62, wherein the temperature-sensitive agent is administered intravenously.

64. The method of claim 61, wherein the temperature-sensitive agent is administered to a specific tissue or organ of the subject.

65. The method of claim 64, wherein the temperature-sensitive agent is administered to the brain and spinal cord by epidural injection.

66. The method of claim 64, wherein the temperature-sensitive agent is administered by percutaneous injection into a target organ.

67. The method of claim 64, wherein the temperature-sensitive agent is administered by endoscopy with an injection needle catheter into a target organ.

68. The method of claim 64, wherein the temperature-sensitive agent is administered by angiocatheter into a target organ.

69. The method of claim 66, wherein the target organ is selected from the group consisting of the liver, kidneys, skeletal muscles, cardiac muscles, pancreas, spleen, heart, brain, spinal cord, skin, eye, lung, intestine, thymus, bone marrow, bone, and cartilage.

70. The method of claim 61, wherein the temperature-sensitive agent is administered by inhalation.

71. The method of claim 61, wherein lowering the body temperature of a subject comprises using a targeted temperature management (TTM) procedure, wherein the TTM procedure comprises application to the subject of one of the group consisting of a cooling catheter, a cooling blanket, and ice.

72. The method of claim 61, wherein normal body temperature of the subject is a non-permissive temperature for the temperature-sensitive agent.

73. The method of claim 61, wherein the temperature-sensitive agent comprises a human ZSCAN4 protein.

74. The method of claim 61, wherein the temperature-sensitive agent comprises a nucleic acid comprising a coding region of human ZSCAN4.

75. The method of claim 74, wherein a temperature-sensitive viral vector comprises the nucleic acid comprising the coding region of human ZSCAN4.

76. The method of claim 75, wherein the temperature-sensitive viral vector is selected from the group consisting of a Sendai virus, an Adeno virus, an Adeno-associated virus, a Retrovirus, and an Alphavirus.

77. The method of claim 75, wherein said temperature-sensitive viral vector is an Alphavirus.

78. The method of claim 77, wherein said Alphavirus is selected from the group consisting of a Venezuelan equine encephalitis virus, a Sindbis virus, and a Semliki Forrest virus.

79. The method of claim 75, wherein the temperature-sensitive viral vector is a Sendai virus.

80. The method of claim 79, wherein the Sendai virus is SeV18+/TS15ΔF.

81. The method of claim 75, wherein the temperature sensitive activity comprises replication and transcription of the temperature-sensitive viral vector.

82. The method of claim 74, wherein a temperature-sensitive self-replicating RNA comprises the nucleic acid comprising the coding region of human ZSCAN4.

83. The method of claim 82, wherein the self-replicating RNA comprises an Alphavirus replicon lacking a viral structural protein coding region.

84. The method of claim 83, wherein the Alphavirus is selected from the group consisting of a Venezuelan equine encephalitis virus, a Sindbis virus, and a Semliki Forrest virus.

85. The method of claim 82, wherein the temperature-sensitive activity comprises one or both of replication and transcription of the temperature-sensitive self-replicating RNA.

86. The method of claim 74, wherein the coding region is operably linked to a promoter.

87. The method of claim 50, wherein the period of time sufficient for the temperature-sensitive activity to produce the therapeutic effect ranges from about 12 hours to about 12 weeks, optionally wherein the period of time is from 1 to 7 days.

88. The method of claim 61, wherein the period of time sufficient to induce the therapeutic effect in the subject is from about 12 hours to about 7 days, optionally wherein the period of time is from about 12 hours to about 72 hours.

89. The method of any one of claims 50-88, wherein the permissive temperature ranges from 30° C. to 36° C., or 31° C. to 35° C., or 32° C. to 34° C.

90. The method of claim 89, wherein the permissive temperature is 33° C.±0.5° C.

91. The method of claim 90, wherein the non-permissive temperature is 37° C.±0.5° C.

92. The method of claim 50, wherein the one or more cells are human cells.

93. The method of claim 92, wherein the one or more human cells are adult stem cells, tissue stem cells, progenitor cells, embryonic stem cells, or induced pluripotent stem cells.

94. The method of claim 92, wherein the one or more human cells are selected from the group consisting of hematopoietic stem cells, mesenchymal stem cells, endothelial stem, cells adipose stem cells, neuronal stem cells, and germ stem cells.

95. The method of claim 92, wherein the one or more human cells are somatic cells, mature cells, or differentiated cells.

96. The method of claim 95, wherein the one or more human cells are selected from the group consisting of epidermal cells, fibroblasts, lymphocytes, hepatocytes, epithelial cells, myocytes, chondrocytes, osteocytes, adipocytes, cardiomyocytes, pancreatic cells, pancreatic p cells, keratinocytes, erythrocytes, peripheral blood mononuclear cells (PBMCs), neurons, glia cells, neurocytes, astrocytes, germ cells, sperm cells, and oocytes.

97. The method of claim 92, wherein the one or more human cells are human bone marrow cells.

98. The method of claim 97, wherein the human bone marrow cells are CD34+ hematopoietic stem cells.

99. The method of claim 98, wherein the human subject suffers from a telomere biology disorder, optionally wherein the subject suffers from bone marrow failure.

100. The method of claim 89, wherein the temperature-sensitive viral vector or the temperature-sensitive self-replicating RNA comprises a nonstructural protein coding region with an insertion of 12-18 nucleotides, wherein the insertion results in expression of a nonstructural Protein 2 (nsP2=helicase proteinase) comprising from 4 to 6 additional amino acids between beta sheet 5 and beta sheet 6 of the nsP2, optionally wherein the additional amino acids result in temperature-sensitivity of the viral vector or the self-replicating RNA.

101. The method of claim 100, wherein the additional amino acids comprise one sequence selected from the group consisting of SEQ ID NO:43 (GCGRT), SEQ ID NO:44 (TGAAA), and SEQ ID NO:45 (LRPHP).

102. The method of claim 100, wherein the additional amino acids comprise the sequence of SEQ ID NO:44 (TGAAA).

103. The method of claim 102, wherein the amino acid sequence of the NsP2 comprises one sequence selected from the group consisting of SEQ ID NOs:29-36.

104. The method of any one of claims 50-103, wherein the amino acid sequence of human ZSCAN4 comprises SEQ ID NO:38 or is at least 95% identical to SEQ ID NO:38.

105. The method of any one of claims 50-103, wherein the amino acid sequence of human ZSCAN4 comprises one of the group consisting of SEQ ID NO:39, SEQ ID NO:40, SEQ ID NO:41, and SEQ ID NO:42, or is at least 95% identical to one of the group consisting of SEQ ID NO:39, SEQ ID NO:40, SEQ ID NO:41, and SEQ ID NO:42.


SEQUENCE LISTING (45 sequences)

SEQ ID NO: 1 - protein, 16 aa, Venezuelan Equine Encephalitis Virus (VEEV)
TLTAKYPGNFTATIEE

SEQ ID NO: 2 - protein, 21 aa, Artificial Sequence
  Synthetic Construct
TLTAKYPGCGRTGNFTATIEE

SEQ ID NO: 3 - protein, 16 aa, Venezuelan Equine Encephalitis Virus (VEEV)
GRVYDMNTGTLRNYDP

SEQ ID NO: 4 - protein, 21 aa, Artificial Sequence
  Synthetic Construct
GRVYDMNTGAAATGTLRNYDP

SEQ ID NO: 5 - protein, 16 aa, Venezuelan Equine Encephalitis Virus (VEEV)
GTLRNYDPRINLVPVN

SEQ ID NO: 6 - protein, 21 aa, Artificial Sequence
  Synthetic Construct
GTLRNYDPLRPHPRINLVPVN

SEQ ID NO: 7 - DNA, 39 bp, Venezuelan Equine Encephalitis Virus (VEEV)
acactgactgccaagtaccctgggaatttcactgccacg

SEQ ID NO: 8 - DNA, 54 bp, Artificial Sequence
  Synthetic Construct
acactgactgccaagtaccctgggtgcggccggactgggaatttcactgccacg

SEQ ID NO: 9 - DNA, 39 bp, Venezuelan Equine Encephalitis Virus (VEEV)
ggaagagtctatgacatgaacactggtacactgcgcaat

SEQ ID NO: 10 - DNA, 54 bp, Artificial Sequence
  Synthetic Construct
ggaagagtctatgacatgaacactggtgccgccgcaactggtacactgcgcaat

SEQ ID NO: 11 - DNA, 39 bp, Venezuelan Equine Encephalitis Virus (VEEV)
ggtacactgcgcaattatgatccgcgcataaacctagta

SEQ ID NO: 12 - DNA, 54 bp, Artificial Sequence
  Synthetic Construct
ggtacactgcgcaattatgatccgctgcggccccatccgcgcataaacctagta

SEQ ID NO: 13 - protein, 13 aa, Venezuelan Equine Encephalitis Virus (VEEV)
TLTAKYPGNFTAT

SEQ ID NO: 14 - protein, 18 aa, Artificial Sequence
  Synthetic Construct
TLTAKYPGCGRTGNFTAT

SEQ ID NO: 15 - protein, 13 aa, Venezuelan Equine Encephalitis Virus (VEEV)
GRVYDMNTGTLRN

SEQ ID NO: 16 - protein, 18 aa, Artificial Sequence
  Synthetic Construct
GRVYDMNTGAAATGTLRN

SEQ ID NO: 17 - protein, 13 aa, Venezuelan Equine Encephalitis Virus (VEEV)
GTLRNYDPRINLV

SEQ ID NO: 18 - protein, 18 aa, Artificial Sequence
  Synthetic Construct
GTLRNYDPLRPHPRINLV

SEQ ID NO: 19 - DNA, 240 bp, Venezuelan Equine Encephalitis Virus (VEEV)
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaa

SEQ ID NO: 20 - DNA, 240 bp, Artificial Sequence
  Synthetic Construct
atgggcggcgcatgagagaagcccagaccaattacctactcaaaatggaaaaagttcacgttgacatcgaagaagacagcccattcctcagagctttgcagcggagttttccgcagtttgaagtagaagccaagcaggtcactgataatgaccatgctaatgcaagagcgttttcgcatcttgcttcaaaactgatcgaaacggaggtggacccatccgacacgatccttgacattggaa

SEQ ID NO: 21 - protein, 13 aa, Venezuelan Equine Encephalitis Virus (VEEV)
GRVYDMNTGTLRN

SEQ ID NO: 22 - protein, 13 aa, Aura virus (Aura)
GDQILPIYGRVSV

SEQ ID NO: 23 - protein, 13 aa, Western equine encephalitis virus (WEEV)
GRVADIRNNTIKD

SEQ ID NO: 24 - protein, 13 aa, Barmah Forest virus (BFV)
GMQIVVTEMRIQR

SEQ ID NO: 25 - protein, 13 aa, O′nyong-nyong virus (ONNV)
NKQICITTRKVDE

SEQ ID NO: 26 - protein, 13 aa, Ross River virus (RRV)
GLQVNVPERKVQP

SEQ ID NO: 27 - protein, 13 aa, Semliki Forest virus (SFV)
GKQAVIAERKIQP

SEQ ID NO: 28 - protein, 13 aa, Sindbis virus (SINV)
GTQLDLQTGRTRV

SEQ ID NO: 29 - protein, 18 aa, Artificial Sequence
  Synthetic Construct
GRVYDMNTGAAATGTLRN

SEQ ID NO: 30 - protein, 18 aa, Artificial Sequence
  Synthetic Construct
GDQILPITGAAAYGRVSV

SEQ ID NO: 31 - protein, 18 aa, Artificial Sequence
  Synthetic Construct
GRVADIRTGAAANNTIKD

SEQ ID NO: 32 - protein, 18 aa, Artificial Sequence
  Synthetic Construct
GMQIVVTTGAAAEMRIQR

SEQ ID NO: 33 - protein, 18 aa, Artificial Sequence
  Synthetic Construct
NKQICITTGAAATRKVDE

SEQ ID NO: 34 - protein, 18 aa, Artificial Sequence
  Synthetic Construct
GLQVNVPTGAAAERKVQP

SEQ ID NO: 35 - protein, 18 aa, Artificial Sequence
  Synthetic Construct
GKQAVIATGAAAERKIQP

SEQ ID NO: 36 - protein, 18 aa, Artificial Sequence
  Synthetic Construct
GTQLDLQTGAAATGRTRV

SEQ ID NO: 37 - protein, 5 aa, Artificial Sequence
  Synthetic Construct
EAAAK

SEQ ID NO: 38 - protein, 433 aa, Homo sapiens
MALDLRTIFQCEPSENNLGSENSAFQQSQGPAVQREEGISEFSRMVLNSFQDSNNSYARQELQRLYRIFHSWLQPEKHSKDEIISLLVLEQFMIGGHCNDKASVKEKWKSSGKNLERFIEDLTDDSINPPALVHVHMQGQEALFSEDMPLRDVIVHLTKQVNAQTTREANMGTPSQTSQDTSLETGQGYEDEQDGWNSSSKTTRVNENITNQGNQIVSLIIIQEENGPRPEEGGVSSDNPYNSKRAELVTARSQEGSINGITFQGVPMVMGAGCISQPEQSSPESALTHQSNEGNSTCEVHQKGSHGVQKSYKCEECPKVFKYLCHLLAHQRRHRNERPFVCPECQKGFFQISDLRVHQIIHTGKKPFTCSMCKKSFSHKTNLRSHERIHTGEKPYTCPFCKTSYRQSSTYHRHMRTHEKITLPSVPSTPEAS

SEQ ID NO: 39 - protein, 311 aa, Homo sapiens
MALDLRTIFQCEPSENNLGSENSAFQQSQGPAVQREEGISEFSRMVLNSFQDSNNSYARQELQRLYRIFHSWLQPEKHSKDEIISLLVLEQFMIGGHCNDKASVKEKWKSSGKNLERFIEDLTDDSINPPALVHVHMQGQEALFSEDMPLRDVIVHLTKQVNAQTTREANMGTPSQTSQDTSLETGQGYEDEQDGWNSSSKTTRVNENITNQGNQIVSLIIIQEENGPRPEEGGVSSDNPYNSKRAELVTARSQEGSINGITFQGVPMVMGAGCISQPEQSSPESALTHQSNEGNSTCEVHQKGSHGVQKS

SEQ ID NO: 40 - protein, 339 aa, Homo sapiens
MALDLRTIFQCEPSENNLGSENSAFQQSQGPAVQREEGISEFSRMVLNSFQDSNNSYARQELQRLYRIFHSWLQPEKHSKDEIISLLVLEQFMIGGHCNDKASVKEKWKSSGKNLERFIEDLTDDSINPPALVHVHMQGQEALFSEDMPLRDVIVHLTKQVNAQTTREANMGTPSQTSQDTSLETGQGYEDEQDGWNSSSKTTRVNENITNQGNQIVSLIIIQEENGPRPEEGGVSSDNPYNSKRAELVTARSQEGSINGITFQGVPMVMGAGCISQPEQSSPESALTHQSNEGNSTCEVHQKGSHGVQKSYKCEECPKVFKYLCHLLAHQRRHRNERP

SEQ ID NO: 41 - protein, 367 aa, Homo sapiens
MALDLRTIFQCEPSENNLGSENSAFQQSQGPAVQREEGISEFSRMVLNSFQDSNNSYARQELQRLYRIFHSWLQPEKHSKDEIISLLVLEQFMIGGHCNDKASVKEKWKSSGKNLERFIEDLTDDSINPPALVHVHMQGQEALFSEDMPLRDVIVHLTKQVNAQTTREANMGTPSQTSQDTSLETGQGYEDEQDGWNSSSKTTRVNENITNQGNQIVSLIIIQEENGPRPEEGGVSSDNPYNSKRAELVTARSQEGSINGITFQGVPMVMGAGCISQPEQSSPESALTHQSNEGNSTCEVHQKGSHGVQKSYKCEECPKVFKYLCHLLAHQRRHRNERPFVCPECQKGFFQISDLRVHQIIHTGKKP

SEQ ID NO: 42 - protein, 395 aa, Homo sapiens
MALDLRTIFQCEPSENNLGSENSAFQQSQGPAVQREEGISEFSRMVLNSFQDSNNSYARQELQRLYRIFHSWLQPEKHSKDEIISLLVLEQFMIGGHCNDKASVKEKWKSSGKNLERFIEDLTDDSINPPALVHVHMQGQEALFSEDMPLRDVIVHLTKQVNAQTTREANMGTPSQTSQDTSLETGQGYEDEQDGWNSSSKTTRVNENITNQGNQIVSLIIIQEENGPRPEEGGVSSDNPYNSKRAELVTARSQEGSINGITFQGVPMVMGAGCISQPEQSSPESALTHQSNEGNSTCEVHQKGSHGVQKSYKCEECPKVFKYLCHLLAHQRRHRNERPFVCPECQKGFFQISDLRVHQIIHTGKKPFTCSMCKKSFSHKTNLRSHERIHTGEKP

SEQ ID NO: 43 - protein, 5 aa, Artificial Sequence
  Synthetic Construct
GCGRT

SEQ ID NO: 44 - protein, 5 aa, Artificial Sequence
  Synthetic Construct
TGAAA

SEQ ID NO: 45 - protein, 5 aa, Artificial Sequence
  Synthetic Construct
LRPHP